SEQUENCE LISTING (21 sequences)

SEQ ID NO: 1 - protein, 536 aa, Artificial Sequence
  Synthetic Construct
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 2 - protein, 621 aa, Artificial Sequence
  Synthetic Construct
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQGGGGAGGGGAGGGGAGGGGAGGGPSLCGGELVDTLQFVCGDRGFYFSRPASRVSARSRGIVEECCFRSCDLALLETYCATPAKSE

SEQ ID NO: 3 - protein, 627 aa, Artificial Sequence
  Synthetic Construct
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQGAPGGGSPAPAPTPAPAPTPAPAGGGPSGAPLCGGELVDTLQFVCGDRGFYFSRPASRVSARSRGIVEECCFRSCDLALLETYCATPAKSE

SEQ ID NO: 4 - protein, 652 aa, Artificial Sequence
  Synthetic Construct
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQGAPGGSPAGSPTSTEEGTSESATPESGPGTSTEPSEGSAPGSPAGSPTSTGPSGAPLCGGELVDTLQFVCGDRGFYFSRPASRVSARSRGIVEECCFRSCDLALLETYCATPAKSE

SEQ ID NO: 5 - protein, 521 aa, Artificial Sequence
  Synthetic Construct
MGIPMGKSMLVLLTFLAFASCCIAARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 6 - DNA, 2275 bp, Homo sapiens
  misc_feature (2005)..(2005): n is a, c, g, or t
gctaacctagtgcctatagctaaggcaggtacctgcatccttgtttttgtttagtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggtttgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcatcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggtggtgcccagggctggaggcccctagaaaaagatcagtaagccccagtgtccccccagcccccatgcttatgtgaacatgcgctgtgtgctgcttgctttggaaactngcctgggtccaggcctagggtgagctcactgtccgtacaaacacaagatcagggctgagggtaaggaaaagaagagactaggaaagctgggcccaaaactggagactgtttgtctttcctagagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccca

SEQ ID NO: 7 - DNA, 1611 bp, Artificial Sequence
  Synthetic Construct
atggaattcagcagccccagccgggaggaatgccccaagcccctgagcagagtgtccatcatggccggatctctgaccggactgctgctgctgcaagccgtgtcttgggccagcggcgccagaccttgcatccccaagagcttcggctacagcagcgtcgtgtgcgtgtgcaacgccacctactgcgacagcttcgacccccctacctttcccgccctgggcaccttcagcagatacgagagcacccgcagcggcagacggatggaactgagcatgggccccatccaggccaatcacaccggaaccggcctgctgctgaccctgcagcccgagcagaaattccagaaagtgaagggcttcggcggagccatgacagacgccgctgccctgaacatcctggccctgagcccccctgctcagaatctgctgctcaagagctacttcagcgaggaaggcatcggctacaacatcatccgggtgcccatggctagctgcgacttcagcatccggacctacacctacgccgacacccccgacgacttccagctgcacaacttcagcctgcccgaagaggacaccaagctgaagatccccctgatccacagagccctgcagctggcccagaggcctgtgtctctgctggctagcccttggaccagccccacctggctgaaaaccaacggagccgtgaacggcaagggcagcctgaagggccagcccggcgacatctaccaccagacctgggcccggtacttcgtgaagttcctggacgcctatgccgagcacaagctgcagttctgggccgtgaccgccgagaatgagcctagcgctggactgctgtccggctaccccttccaatgcctgggcttcacacccgagcaccagcgggactttatcgccagagatctgggccccacactggccaacagcacccaccacaacgtgcggctgctgatgctggacgaccagagactgctgctcccccactgggccaaggtggtgctgacagaccccgaggccgccaaatacgtccacgggattgccgtgcactggtatctggactttctggcccctgccaaggccacactgggcgagacacaccggctgttccccaacaccatgctgttcgccagcgaggcctgcgtgggcagcaagttctgggagcagagcgtgcggctgggcagctgggacagaggcatgcagtacagccacagcatcatcaccaacctgctgtaccacgtcgtgggctggaccgactggaatctggccctgaaccctgagggaggacccaactgggtccgcaacttcgtggacagccccatcatcgtggacatcaccaaggacaccttctacaagcagcccatgttctaccacctgggccacttcagcaagttcatccccgagggcagccagagagtgggactggtcgccagccagaagaacgatctggacgccgtggccctgatgcaccctgatggcagcgctgtggtggtggtcctgaatcggtccagcaaggacgtgcccctgaccatcaaggaccccgccgtgggcttcctggaaaccatcagccccggctactccatccacacctacctgtggcggagacaatga

SEQ ID NO: 8 - DNA, 1869 bp, Artificial Sequence
  Synthetic Construct
atggaattcagcagccccagccgggaggaatgccccaagcccctgagcagagtgtccatcatggccggatctctgaccggactgctgctgctgcaagccgtgtcttgggccagcggcgccagaccttgcatccccaagagcttcggctacagcagcgtcgtgtgcgtgtgcaacgccacctactgcgacagcttcgacccccctacctttcccgccctgggcaccttcagcagatacgagagcacccgcagcggcagacggatggaactgagcatgggccccatccaggccaatcacaccggaaccggcctgctgctgaccctgcagcccgagcagaaattccagaaagtgaagggcttcggcggagccatgacagacgccgctgccctgaacatcctggccctgagcccccctgctcagaatctgctgctcaagagctacttcagcgaggaaggcatcggctacaacatcatccgggtgcccatggctagctgcgacttcagcatccggacctacacctacgccgacacccccgacgacttccagctgcacaacttcagcctgcccgaagaggacaccaagctgaagatccccctgatccacagagccctgcagctggcccagaggcctgtgtctctgctggctagcccttggaccagccccacctggctgaaaaccaacggagccgtgaacggcaagggcagcctgaagggccagcccggcgacatctaccaccagacctgggcccggtacttcgtgaagttcctggacgcctatgccgagcacaagctgcagttctgggccgtgaccgccgagaatgagcctagcgctggactgctgtccggctaccccttccaatgcctgggcttcacacccgagcaccagcgggactttatcgccagagatctgggccccacactggccaacagcacccaccacaacgtgcggctgctgatgctggacgaccagagactgctgctcccccactgggccaaggtggtgctgacagaccccgaggccgccaaatacgtccacgggattgccgtgcactggtatctggactttctggcccctgccaaggccacactgggcgagacacaccggctgttccccaacaccatgctgttcgccagcgaggcctgcgtgggcagcaagttctgggagcagagcgtgcggctgggcagctgggacagaggcatgcagtacagccacagcatcatcaccaacctgctgtaccacgtcgtgggctggaccgactggaatctggccctgaaccctgagggaggacccaactgggtccgcaacttcgtggacagccccatcatcgtggacatcaccaaggacaccttctacaagcagcccatgttctaccacctgggccacttcagcaagttcatccccgagggcagccagagagtgggactggtcgccagccagaagaacgatctggacgccgtggccctgatgcaccctgatggcagcgctgtggtggtggtcctgaatcggtccagcaaggacgtgcccctgaccatcaaggaccccgccgtgggcttcctggaaaccatcagccccggctactccatccacacctacctgtggcggagacaaggcggaggcggagctggtggcggcggagcaggcggtggtggtgcaggcggcggaggtgctggcggaggaccatctctttgtggcggagaactggtggacaccctgcagttcgtgtgtggcgacagaggcttctactttagcagacccgccagcagagtgtccgccagatctagaggaatcgtggaagagtgctgcttcagaagctgcgacctggcactgctggaaacctactgtgccacaccagccaagagcgagtgatga

SEQ ID NO: 9 - DNA, 1887 bp, Artificial Sequence
  Synthetic Construct
atggaattcagcagccccagccgggaggaatgccccaagcccctgagcagagtgtccatcatggccggatctctgaccggactgctgctgctgcaagccgtgtcttgggccagcggcgccagaccttgcatccccaagagcttcggctacagcagcgtcgtgtgcgtgtgcaacgccacctactgcgacagcttcgacccccctacctttcccgccctgggcaccttcagcagatacgagagcacccgcagcggcagacggatggaactgagcatgggccccatccaggccaatcacaccggaaccggcctgctgctgaccctgcagcccgagcagaaattccagaaagtgaagggcttcggcggagccatgacagacgccgctgccctgaacatcctggccctgagcccccctgctcagaatctgctgctcaagagctacttcagcgaggaaggcatcggctacaacatcatccgggtgcccatggctagctgcgacttcagcatccggacctacacctacgccgacacccccgacgacttccagctgcacaacttcagcctgcccgaagaggacaccaagctgaagatccccctgatccacagagccctgcagctggcccagaggcctgtgtctctgctggctagcccttggaccagccccacctggctgaaaaccaacggagccgtgaacggcaagggcagcctgaagggccagcccggcgacatctaccaccagacctgggcccggtacttcgtgaagttcctggacgcctatgccgagcacaagctgcagttctgggccgtgaccgccgagaatgagcctagcgctggactgctgtccggctaccccttccaatgcctgggcttcacacccgagcaccagcgggactttatcgccagagatctgggccccacactggccaacagcacccaccacaacgtgcggctgctgatgctggacgaccagagactgctgctcccccactgggccaaggtggtgctgacagaccccgaggccgccaaatacgtccacgggattgccgtgcactggtatctggactttctggcccctgccaaggccacactgggcgagacacaccggctgttccccaacaccatgctgttcgccagcgaggcctgcgtgggcagcaagttctgggagcagagcgtgcggctgggcagctgggacagaggcatgcagtacagccacagcatcatcaccaacctgctgtaccacgtcgtgggctggaccgactggaatctggccctgaaccctgagggaggacccaactgggtccgcaacttcgtggacagccccatcatcgtggacatcaccaaggacaccttctacaagcagcccatgttctaccacctgggccacttcagcaagttcatccccgagggcagccagagagtgggactggtcgccagccagaagaacgatctggacgccgtggccctgatgcaccctgatggcagcgctgtggtggtggtcctgaatcggtccagcaaggacgtgcccctgaccatcaaggaccccgccgtgggcttcctggaaaccatcagccccggctactccatccacacctacctgtggcggagacaaggagcaccaggcggaggatctccagctcctgctcctacaccagctccagcaccgacgcctgctccagctggcggaggaccttctggtgcacctctttgtggcggagagctggtggataccctgcagttcgtgtgtggcgaccggggcttctactttagcagacctgccagcagagtgtccgccagatctagaggcatcgtggaagagtgctgcttcagaagctgcgacctggcactgctggaaacctactgtgccacaccagccaagagcgagtgatga

SEQ ID NO: 10 - DNA, 1962 bp, Artificial Sequence
  Synthetic Construct
atggaattcagcagccccagccgggaggaatgccccaagcccctgagcagagtgtccatcatggccggatctctgaccggactgctgctgctgcaagccgtgtcttgggccagcggcgccagaccttgcatccccaagagcttcggctacagcagcgtcgtgtgcgtgtgcaacgccacctactgcgacagcttcgacccccctacctttcccgccctgggcaccttcagcagatacgagagcacccgcagcggcagacggatggaactgagcatgggccccatccaggccaatcacaccggaaccggcctgctgctgaccctgcagcccgagcagaaattccagaaagtgaagggcttcggcggagccatgacagacgccgctgccctgaacatcctggccctgagcccccctgctcagaatctgctgctcaagagctacttcagcgaggaaggcatcggctacaacatcatccgggtgcccatggctagctgcgacttcagcatccggacctacacctacgccgacacccccgacgacttccagctgcacaacttcagcctgcccgaagaggacaccaagctgaagatccccctgatccacagagccctgcagctggcccagaggcctgtgtctctgctggctagcccttggaccagccccacctggctgaaaaccaacggagccgtgaacggcaagggcagcctgaagggccagcccggcgacatctaccaccagacctgggcccggtacttcgtgaagttcctggacgcctatgccgagcacaagctgcagttctgggccgtgaccgccgagaatgagcctagcgctggactgctgtccggctaccccttccaatgcctgggcttcacacccgagcaccagcgggactttatcgccagagatctgggccccacactggccaacagcacccaccacaacgtgcggctgctgatgctggacgaccagagactgctgctcccccactgggccaaggtggtgctgacagaccccgaggccgccaaatacgtccacgggattgccgtgcactggtatctggactttctggcccctgccaaggccacactgggcgagacacaccggctgttccccaacaccatgctgttcgccagcgaggcctgcgtgggcagcaagttctgggagcagagcgtgcggctgggcagctgggacagaggcatgcagtacagccacagcatcatcaccaacctgctgtaccacgtcgtgggctggaccgactggaatctggccctgaaccctgagggaggacccaactgggtccgcaacttcgtggacagccccatcatcgtggacatcaccaaggacaccttctacaagcagcccatgttctaccacctgggccacttcagcaagttcatccccgagggcagccagagagtgggactggtcgccagccagaagaacgatctggacgccgtggccctgatgcaccctgatggcagcgctgtggtggtggtcctgaatcggtccagcaaggacgtgcccctgaccatcaaggaccccgccgtgggcttcctggaaaccatcagccccggctactccatccacacctacctgtggcggagacaaggagcaccaggcggatctccagcaggatctccaacctctaccgaggaaggcacaagcgagtctgccacacctgagtctggacctggcacaagcacagagcctagcgaaggatctgccccaggttctcctgccggctctcctacaagtacaggaccttctggcgctccactgtgtggcggagaactggtggataccctgcagttcgtgtgcggcgacagaggcttctactttagcagacccgccagcagagtgtccgccagatctagaggaatcgtggaagagtgctgcttcagaagctgcgatctggcactgctggaaacctactgtgccacaccagccaagagcgagtgatga

SEQ ID NO: 11 - DNA, 1566 bp, Artificial Sequence
  Synthetic Construct
atggggattcctatgggcaagtctatgctggtcctgctgacatttctggccttcgcttcatgctgtatcgctgccagaccttgcatccccaagagcttcggctacagcagcgtcgtgtgcgtgtgcaacgccacctactgcgacagcttcgacccccctacctttcccgccctgggcaccttcagcagatacgagagcacccgcagcggcagacggatggaactgagcatgggccccatccaggccaatcacaccggaaccggcctgctgctgaccctgcagcccgagcagaaattccagaaagtgaagggcttcggcggagccatgacagacgccgctgccctgaacatcctggccctgagcccccctgctcagaatctgctgctcaagagctacttcagcgaggaaggcatcggctacaacatcatccgggtgcccatggctagctgcgacttcagcatccggacctacacctacgccgacacccccgacgacttccagctgcacaacttcagcctgcccgaagaggacaccaagctgaagatccccctgatccacagagccctgcagctggcccagaggcctgtgtctctgctggctagcccttggaccagccccacctggctgaaaaccaacggagccgtgaacggcaagggcagcctgaagggccagcccggcgacatctaccaccagacctgggcccggtacttcgtgaagttcctggacgcctatgccgagcacaagctgcagttctgggccgtgaccgccgagaatgagcctagcgctggactgctgtccggctaccccttccaatgcctgggcttcacacccgagcaccagcgggactttatcgccagagatctgggccccacactggccaacagcacccaccacaacgtgcggctgctgatgctggacgaccagagactgctgctcccccactgggccaaggtggtgctgacagaccccgaggccgccaaatacgtccacgggattgccgtgcactggtatctggactttctggcccctgccaaggccacactgggcgagacacaccggctgttccccaacaccatgctgttcgccagcgaggcctgcgtgggcagcaagttctgggagcagagcgtgcggctgggcagctgggacagaggcatgcagtacagccacagcatcatcaccaacctgctgtaccacgtcgtgggctggaccgactggaatctggccctgaaccctgagggaggacccaactgggtccgcaacttcgtggacagccccatcatcgtggacatcaccaaggacaccttctacaagcagcccatgttctaccacctgggccacttcagcaagttcatccccgagggcagccagagagtgggactggtcgccagccagaagaacgatctggacgccgtggccctgatgcaccctgatggcagcgctgtggtggtggtcctgaatcggtccagcaaggacgtgcccctgaccatcaaggaccccgccgtgggcttcctggaaaccatcagccccggctactccatccacacctacctgtggcggagacaatga

SEQ ID NO: 12 - protein, 67 aa, Artificial Sequence
  Synthetic Construct
AYRPSETLCGGELVDTLQFVCGDRGFYFSRPASRVSRRSRGIVEECCFRSCDLALLETYCATPAKSE

SEQ ID NO: 13 - protein, 85 aa, Artificial Sequence
  Synthetic Construct
GGGGAGGGGAGGGGAGGGGAGGGPSLCGGELVDTLQFVCGDRGFYFSRPASRVSARSRGIVEECCFRSCDLALLETYCATPAKSE

SEQ ID NO: 14 - protein, 91 aa, Artificial Sequence
  Synthetic Construct
GAPGGGSPAPAPTPAPAPTPAPAGGGPSGAPLCGGELVDTLQFVCGDRGFYFSRPASRVSARSRGIVEECCFRSCDLALLETYCATPAKSE

SEQ ID NO: 15 - protein, 116 aa, Artificial Sequence
  Synthetic Construct
GAPGGSPAGSPTSTEEGTSESATPESGPGTSTEPSEGSAPGSPAGSPTSTGPSGAPLCGGELVDTLQFVCGDRGFYFSRPASRVSARSRGIVEECCFRSCDLALLETYCATPAKSE

SEQ ID NO: 16 - protein, 24 aa, Artificial Sequence
  Synthetic Construct
MGIPMGKSMLVLLTFLAFASCCIA

SEQ ID NO: 17 - DNA, 261 bp, Artificial Sequence
  Synthetic Construct
ggcggaggcggagctggtggcggcggagcaggcggtggtggtgcaggcggcggaggtgctggcggaggaccatctctttgtggcggagaactggtggacaccctgcagttcgtgtgtggcgacagaggcttctactttagcagacccgccagcagagtgtccgccagatctagaggaatcgtggaagagtgctgcttcagaagctgcgacctggcactgctggaaacctactgtgccacaccagccaagagcgagtgatga

SEQ ID NO: 18 - DNA, 279 bp, Artificial Sequence
  Synthetic Construct
ggagcaccaggcggaggatctccagctcctgctcctacaccagctccagcaccgacgcctgctccagctggcggaggaccttctggtgcacctctttgtggcggagagctggtggataccctgcagttcgtgtgtggcgaccggggcttctactttagcagacctgccagcagagtgtccgccagatctagaggcatcgtggaagagtgctgcttcagaagctgcgacctggcactgctggaaacctactgtgccacaccagccaagagcgagtgatga

SEQ ID NO: 19 - DNA, 354 bp, Artificial Sequence
  Synthetic Construct
ggagcaccaggcggatctccagcaggatctccaacctctaccgaggaaggcacaagcgagtctgccacacctgagtctggacctggcacaagcacagagcctagcgaaggatctgccccaggttctcctgccggctctcctacaagtacaggaccttctggcgctccactgtgtggcggagaactggtggataccctgcagttcgtgtgcggcgacagaggcttctactttagcagacccgccagcagagtgtccgccagatctagaggaatcgtggaagagtgctgcttcagaagctgcgatctggcactgctggaaacctactgtgccacaccagccaagagcgagtgatga

SEQ ID NO: 20 - DNA, 72 bp, Artificial Sequence
  Synthetic Construct
atggggattcctatgggcaagtctatgctggtcctgctgacatttctggccttcgcttcatgctgtatcgct

SEQ ID NO: 21 - protein, 317 aa, Artificial Sequence [sequence omitted]

(19) United States
(12) Patent Application Publication
(10) Pub. No.: US 2021/0000929 A1
(43) Pub. Date: Jan. 7, 2021

(54) COMPOSITIONS AND METHODS FOR TREATING PARKINSON'S DISEASE

(71) Applicant: AvroBio, Inc., Cambridge, MA (US)

(72) Inventors: Chris MASON, Cambridge, MA (US); Nico Peter VAN TIL, Leiden (NL); Oliver COOPER, Boston, MA (US)

(21) Appl. No.: 16/979,378

(22) PCT Filed: Mar. 8, 2019
(86) PCT No.: PCT/US19/21422

§ 371 Date: Sep. 9, 2020

Related U.S. Application Data
(60) Provisional application No. 62/641,012, filed on Mar. 9, 2018.

(51) Int. Cl. A61K 38/47; C12N 9/38; A61K 35/28; C07K 14/81; C07K 14/65; C07K 14/775; C07K 14/54; A61K 38/20; A61P 25/16; C12N 15/113; A61K 9/00; C12N 15/86; A61K 31/505; A61K 31/4439; A61K 31/138

(52) U.S. Cl. CPC A61K 38/47; C12N 2740/13043; C12Y 302/01045; A61K 35/28; C07K 14/8125; C07K 14/65; C07K 14/775; C07K 14/54; A61K 38/20; A61P 25/16; C12N 15/113; A61K 9/0019; A61K 9/0085; C12N 15/86; A61K 31/505; A61K 31/4439; A61K 31/138; C07K 2319/02; C07K 2319/055; C07K 2319/06; C12N 2310/141; C12N 9/2468

(57) ABSTRACT

Described herein are methods for treating a subject having or at risk of developing Parkinson's disease, by administering pluripotent cells that express glucocerebrosidase (GBA) or pluripotent cells that express GBA and one or more M2-promoting agents to the subject. Also disclosed are compositions comprising pluripotent cells expressing GBA, such as pluripotent cells expressing GBA and one or more M2-promoting agents.

### SEQUENCE LISTING

The instant application contains a Sequence Listing which has been submitted electronically in ASCII format and is hereby incorporated by reference in its entirety. Said ASCII copy, created on Mar. 8, 2019 is named 51182-007WO2_Sequence Listing_3.8.19_ST25 and is 49, 962 bytes in size.

### FIELD OF THE INVENTION

The invention relates to compositions and methods for treating Parkinson's disease.

### BACKGROUND

Parkinson's disease (PD) is a progressive disorder of the nervous system that affects movement and produces symptoms such as resting tremor, rigidity, and bradykinesia. Patients suffering from Parkinson's disease may also experience non-motor symptoms, including depression, constipation, pain, sleep disorders, cognitive decline, and olfactory dysfunction. Post-mortem analyses of PD patient brains often reveal Lewy bodies containing α-synuclein in affected brain regions and a loss of dopaminergic neurons in the substantia nigra pars compacta. Current treatments for PD primarily focus on increasing dopamine levels. There remains a need for improved therapeutic modalities for the treatment of PD.

### SUMMARY OF THE INVENTION

The present invention provides methods for treating Parkinson's disease using pluripotent cells, such as CD34+ cells and hematopoietic stem cells, among others, expressing glucocerebrosidase (GBA). The cells may be administered to a patient having Parkinson's disease by one or more of a variety of routes, including directly to the central nervous system of the patient (e.g., by intracerebroventricular administration) or systemically (e.g., by intravenous administration), among others. The cells may further express one or more additional transgenes, such as a transgene encoding an M2-promoting agent. The invention also features compositions containing such pluripotent cells, as well as kits containing these cells for the treatment of Parkinson's disease.

In a first aspect, the invention provides a method of treating Parkinson's disease in a subject by administering to the subject a composition containing a population of pluripotent cells that express a transgene encoding GBA. In some embodiments, the GBA is full-length GBA, such as GBA having an amino acid sequence of SEQ ID NO. 1 or a variant thereof having at least 85% sequence identity thereto (e.g., 85%, 86%, 87%, 88%, 89%, 90%, 91%, 92%, 93%, 94%, 95%, 96%, 97%, 98%, 99%, or 100% sequence identity to SEQ ID NO. 1). In some embodiments, the GBA is mature GBA protein. In some embodiments, the GBA is a catalytic domain of GBA, such as a catalytic domain of SEQ ID NO. 1 (e.g., a catalytic domain containing residues 76-381 and 416-430 of SEQ ID NO. 1.

In some embodiments, the transgene encoding GBA contains a polynucleotide encoding wild-type human GBA (SEQ ID NO. 1). In some embodiments, the transgene encoding GBA includes a polynucleotide encoding a polypeptide having at least 85% sequence identity (e.g., at least 85%, 90%, 95%, 96%, 97%, 98%, 99%, or more, sequence identity) to a polypeptide having the amino acid sequence of SEQ ID NO. 1. In some embodiments, the transgene encoding GBA includes a polynucleotide encoding polypeptide that contains one or more amino acid substitutions, such as one or more conservative amino acid substitutions, relative to a polypeptide having the sequence of SEQ ID NO. 1 (e.g., 1, 2, 3, 4, 5, 6, 7, 8, 9, or 10 or more amino acid substitutions, such as 1, 2, 3, 4, 5, 6, 7, 8, 9, or 10 or more conservative amino acid substitutions). In some embodiments, the transgene encoding GBA contains a polynucleotide having at least 85% sequence identity (e.g., at least 85%, 90%, 95%, 96%, 97%, 98%, 99%, or more, sequence identity) to the nucleic acid sequence of SEQ ID NO. 6. In some embodiments, the transgene encoding GBA contains a codon-optimized polynucleotide having at least 85% sequence identity (e.g., at least 85%, 90%, 95%, 96%, 97%, 98%, 99%, or more, sequence identity) to the nucleic acid sequence of SEQ ID NO. 7.

In some embodiments, the transgene encoding GBA encodes non-secreted GBA. In some embodiments, the transgene encoding non-secreted GBA has been codon-optimized. In some embodiments, the transgene encoding secreted GBA comprises a signal peptide, such as a GBA signal peptide (e.g., a 39-amino acid GBA signal peptide). In some embodiments, the transgene encoding secreted GBA comprises a modified signal peptide. In some embodiments, the GBA comprising a modified signal peptide has an amino acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the amino acid sequence of SEQ ID NO. 5. In some embodiments, the transgene encoding secreted GBA comprising a modified signal peptide has a nucleic acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the nucleic acid sequence of SEQ ID NO. 11. In some embodiments, the modified signal peptide has an amino acid sequence having at least 85% sequence identity to the amino acid sequence of SEQ ID NO. 16. In some embodiments, the modified signal peptide is encoded by a polynucleotide having at least 85% sequence identity (e.g., 85%, 86%, 87%, 88%, 89%, 90%, 91%, 92%, 93%, 94%, 95%, 96%, 97%, 98%, 99%, or 100% sequence identity) to the nucleic acid sequence of SEQ ID NO. 20.

In some embodiments, the transgene encoding GBA encodes secreted GBA. In some embodiments, the transgene encoding secreted GBA has been codon-optimized. In some embodiments, the transgene encoding secreted GBA comprises a secretory signal peptide. In some embodiments, the secretory signal peptide is a GBA secretory signal peptide. In some embodiments, the secretory signal peptide is an alpha-1 antitrypsin secretory signal peptide. In some embodiments, the secretory signal peptide is an insulin-like growth factor II (IGF-II) secretory signal peptide.

In some embodiments, the GBA is a GBA fusion protein. In some embodiments, the transgene encoding GBA encodes a GBA fusion protein. In some embodiments, the GBA fusion protein has an amino acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the amino acid sequence of SEQ ID NO. 2. In some embodiments, the GBA fusion protein has an amino acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the amino acid sequence of SEQ ID NO. 3. In some embodiments, the GBA fusion protein has an amino acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the amino acid sequence of SEQ ID NO. 4. In some embodiments, the transgene encoding the GBA fusion protein has a nucleic acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the nucleic acid sequence of SEQ ID NO. 8. In some embodiments, the transgene encoding the GBA fusion protein has a nucleic acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the nucleic acid sequence of SEQ ID NO. 9. In some embodiments, the transgene encoding the GBA fusion protein has a nucleic acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the nucleic acid sequence of SEQ ID NO. 10. In some embodiments, the GBA fusion protein contains GBA and a glycosylation independent lysosomal targeting (GILT) tag. In some embodiments, the GILT tag is operably linked to the N-terminus of the GBA. In some embodiments, the GILT tag is operably linked to the C-terminus of the GBA. In some embodiments, the GILT tag contains a human IGF-II mutein having an amino acid sequence at least 70% identical to the amino acid sequence of mature human IGF-II (SEQ ID NO. 12). The mutein may have diminished binding affinity for the insulin receptor relative to the affinity of naturally-occurring human IGF-II for the insulin receptor, and/or may be resistant to furin cleavage. The mutein may bind to the human cation-independent mannose-6-phosphate receptor in a mannose-6-phosphate-independent manner. In some embodiments, the IGF-II mutein contains a mutation within a region corresponding to amino acids 30-40 of SEQ ID NO. 12, and wherein the mutation abolishes at least one furin protease cleavage site. In some embodiments, the mutation is an amino acid substitution, deletion, and/or insertion. In some embodiments, the mutation is a Lys or Ala amino acid substitution at a position corresponding to Arg37 or Arg40 of SEQ ID NO. 12. In some embodiments, the mutation is a deletion or replacement of amino acid residues corresponding to positions selected form the group consisting of 31-40, 32-40, 33-40, 34-40, 30-39, 31-39, 32-39, 34-37, 33-39, 35-39, 36-39, 37-40, 34-40 of SEQ ID NO. 12, and combinations thereof. In some embodiments, the GILT tag has an amino acid sequence having at least 70% sequence identity (e.g., 70%, 75%, 80%, 85%, 90%, 95%, 96%, 97%, 98%, 99%, or greater, sequence identity) to the amino acid sequence of SEQ NO. 13. In some embodiments, the GILT tag has an amino acid sequence having at least 70% sequence identity (e.g., 70%, 75%, 80%, 85%, 90%, 95%, 96%, 97%, 98%, 99%, or greater, sequence identity) to the amino acid sequence of SEQ NO. 14. In some embodiments, the GILT tag has an amino acid sequence having at least 70% sequence identity (e.g., 70%, 75%, 80%, 85%, 90%, 95%, 96%, 97%, 98%, 99%, or greater, sequence identity) to the amino acid sequence of SEQ NO. 15. In some embodiments, the GILT tag has a nucleic acid sequence having at least 85% sequence identity (e.g., 85%, 90%, 95%, 96%, 97%, 98%, 99%, or greater, sequence identity) to the nucleic acid sequence of SEQ ID NO. 17. In some embodiments, the GILT tag has a nucleic acid sequence having at least 85% sequence identity (e.g., 85%, 90%, 95%, 96%, 97%, 98%, 99%, or greater, sequence identity) to the nucleic acid sequence of SEQ ID NO. 18. In some embodiments, the GILT tag has a nucleic acid sequence having at least 85% sequence identity (e.g., 85%, 90%, 95%, 96%, 97%, 98%, 99%, or greater, sequence identity) to the nucleic acid sequence of SEQ ID NO. 19.

In some embodiments, the GBA fusion protein contains a low-density lipoprotein receptor family (LDLRf) binding (Rb) domain of apolipoprotein E (ApoE), or a fragment, variant, or oligomer thereof. In some embodiments, the Rb domain of ApoE, or a fragment, variant, or oligomer thereof, is operably linked to the N-terminus of the GBA. In some embodiments, the Rb domain of ApoE, or a fragment, variant, or oligomer thereof is operably linked to the C-terminus of the GBA. In some embodiments, the secreted GBA fusion protein contains 1 or more (e.g., 1, 2, 3, 4, 5, 6, 7, 8, 9, 10, or more) oligomers of the Rb domain of ApoE. In some embodiments, the Rb domain contains a region of ApoE having at least 70% sequence identity (e.g., 70%, 75%, 80%, 85%, 90%, 95%, 96%, 97%, 98%, 99%, or greater, sequence identity) to residues 25-185 of SEQ ID NO. 21. In some embodiments, the Rb domain contains a region of ApoE having at least 70% sequence identity (e.g., 70%, 75%, 80%, 85%, 90%, 95%, 96%, 97%, 98%, 99%, or greater, sequence identity) to residues 50-180 of SEQ ID NO. 21. In some embodiments, the Rb domain contains a region of ApoE having at least 70% sequence identity (e.g., 70%, 75%, 80%, 85%, 90%, 95%, 96%, 97%, 98%, 99%, or greater, sequence identity) to residues 75-175 of SEQ ID NO. 21. In some embodiments, the Rb domain contains a region of ApoE having at least 70% sequence identity (e.g., 70%, 75%, 80%, 85%, 90%, 95%, 96%, 97%, 98%, 99%, or greater, sequence identity) to residues 100-170 of SEQ ID NO. 21. In some embodiments, the Rb domain contains a region of ApoE having at least 70% sequence identity (e.g., 70%, 75%, 80%, 85%, 90%, 95%, 96%, 97%, 98%, 99%, or greater, sequence identity) to residues 125-160 of SEQ ID NO. 21. In some embodiments, the Rb domain contains a region of ApoE having at least 70% sequence identity (e.g., 70%, 75%, 80%, 85%, 90%, 95%, 96%, 97%, 98%, 99%, or greater, sequence identity) to residues 130-150 of SEQ ID NO. 21. In some embodiments, the Rb domain contains a region of ApoE having at least 70% sequence identity (e.g., 70%, 75%, 80%, 85%, 90%, 95%, 96%, 97%, 98%, 99%, or greater, sequence identity) to residues 148-173 or a portion thereof containing residues 159-167 of SEQ ID NO. 21, or a variant having at least 70% sequence identity (e.g., 70%, 75%, 80%, 85%, 90%, 95%, 96%, 97%, 98%, 99%, or greater, sequence identity) to residues 159-167 of SEQ ID NO. 21). In some embodiments, the Rb domain contains a region having at least 70% sequence identity (e.g., 70%, 75%, 80%, 85%, 90%, 95%, 96%, 97%, 98%, 99%, or greater, sequence identity) to the amino acid sequence of residues 159-167 of SEQ ID NO. 21.

In some embodiments, the transgene encoding GBA further contains a micro RNA (miRNA) targeting sequence (e.g., a miR-126 targeting sequence). In some embodiments, the miRNA targeting sequence (e.g., a miR-126 targeting sequence) is located within the 3′-UTR of the transgene.

In some embodiments, the secreted GBA penetrates the blood brain barrier (BBB) in the subject.

In some embodiments, the Parkinson's disease is GBA-associated Parkinson's disease.

In some embodiments, the pluripotent cells are CD34+ cells. In some embodiments, the CD34+ cells are embryonic stem cells. In some embodiments, the CD34+ cells are induced pluripotent stem cells. In some embodiments, the CD34+ cells are hematopoietic stem cells. In some embodiments, the CD34+ cells are myeloid progenitor cells.

In some embodiments, a population of endogenous microglia in the subject has been ablated prior to administration of the composition to the subject. In some embodiments, the method includes ablating a population of endogenous microglia in the subject prior to administering the composition to the subject. In some embodiments, the microglia are ablated using an agent selected from the group consisting of busulfan, PLX3397, PLX647, PLX5622, treosulfan, and clodronate liposomes, by radiation therapy, or a combination thereof.

In some embodiments, the composition is administered systemically to the subject. In some embodiments, the composition is administered to the subject by way of intravenous injection. In some embodiments, the composition is administered directly to the central nervous system of the subject. In some embodiments, is administered to the subject by way of intracerebroventricular injection, stereotactic injection, or a combination thereof.

In some embodiments, the composition is administered directly to the bone marrow of the subject. In some embodiments, the composition is administered to the subject by way of intraosseous injection.

In some embodiments, the composition is administered to the subject by way of a bone marrow transplant. In some embodiments, the composition is administered to the subject by way of intracerebroventricular injection. In some embodiments, the composition is administered to the subject by way of intravenous injection.

In some embodiments, the composition is administered to the subject by direct administration to the central nervous system of the subject and by systemic administration. In some embodiments, the composition is administered to the subject by way of intracerebroventricular injection and intravenous injection.

In some embodiments, the method includes administering to the subject a population of CD34+ cells. In some embodiments, the population of CD34+ cells is administered to the subject prior to administration of the composition. In some embodiments, the population of CD34+ cells is administered to the subject following administration of the composition. In some embodiments, the CD34+ cells are selected from the group consisting of embryonic stem cells, induced pluripotent stem cells, hematopoietic stem cells, and myeloid progenitor cells. In some embodiments, the CD34+ cells are not modified to express a transgene encoding GBA. In some embodiments, the CD34+ cells are administered to the subject systemically. In some embodiments, the CD34+ cells are administered to the subject by way of intravenous injection.

In some embodiments, endogenous GBA is disrupted in the pluripotent cells prior to administration of the composition to the subject.

In some embodiments, the endogenous GBA is disrupted by contacting the pluripotent cells with a nuclease that catalyzes cleavage of an endogenous GBA nucleic acid in the pluripotent cells. In some embodiments, the nuclease is a CRISPR-associated protein. In some embodiments, the CRISPR-associated protein is CRISPR associated protein 9. In some embodiments, the nuclease is a transcription activator-like effector nuclease, a meganuclease, or a zinc finger nuclease.

In some embodiments, the endogenous GBA is disrupted by contacting the pluripotent cells with an inhibitory RNA molecule, e.g., for a time and in a quantity sufficient to disrupt expression of the endogenous GBA. In some embodiments, the inhibitory RNA molecule is a short interfering RNA (siRNA), a short hairpin RNA (shRNA), or a micro RNA (miRNA).

In some embodiments, the endogenous GBA is disrupted in the subject prior to administration of the composition to the subject. In some embodiments, the endogenous GBA is disrupted by administering to the subject an inhibitory RNA molecule. In some embodiments, the inhibitory RNA molecule is a siRNA, a shRNA, or a miRNA.

In some embodiments, the endogenous GBA is disrupted in a population of neurons in the subject prior to administration of the composition to the subject. In some embodiments, the endogenous GBA is disrupted in a population of neurons by contacting the population of neurons with an inhibitory RNA molecule, e.g., for a time and in a quantity sufficient to disrupt expression of the endogenous GBA. In some embodiments, the inhibitory RNA molecule is a siRNA, a shRNA, or a miRNA.

In some embodiments, the pluripotent cells that express a transgene encoding GBA further express one or more transgenes that each encode an M2-promoting agent. In some embodiments, the pluripotent cells that express a transgene encoding GBA express two transgenes that each encode an M2-promoting agent.

In some embodiments, the one or more transgenes that each encode an M2-promoting agent encodes a cytokine selected from the group including interleukin-25 (IL-25), interleukin-4 (IL-4), interleukin-10 (IL-10), interleukin-13 (IL-13), and transforming growth factor beta (TGF-β). In some embodiments, the one or more transgenes that each encode an M2-promoting agent encodes IL-25. In some embodiments, the one or more transgenes that each encode an M2-promoting agent encodes an agent selected from the group containing a glucocorticoid receptor, a peroxisome proliferator-activated receptor (PPAR), PPARγ, PPAR β/δ, an estrogen receptor, nuclear receptor subfamily 4 group A member 2 (NR4A2), lysine demethylase 6B (KDM6B), MSH homeobox 3 (MSX3), family with sequence similarity 19 (chemokine (C—C motif)-like), member A3 (FAM19A3), nuclear factor NF-Kappa-B P50 subunit (NF-κB p50), microRNA 124 (miR124), microRNA 21 (miR21), and microRNA 181c (miR181c), C-X3-C motif chemokine ligand 1 (CX3CL1), C-X3-C motif chemokine receptor 1 (CX3CR1), CD200 molecule (CD200), CD200 receptor 1 (CD200R), complement factor H (CFH), leukocyte surface antigen CD47 (CD47), complement decay-accelerating factor (CD55), trophoblast leukocyte common antigen (CD46), adhesion G protein-coupled receptor E5 (ADGRE5), signal regulatory protein alpha (SIRPA), and siglecs.

In some embodiments, the pluripotent cells are autologous cells. In some embodiments, the pluripotent cells are allogeneic cells.

In some embodiments, the pluripotent cells are transduced ex vivo to express the GBA. In some embodiments, the pluripotent cells are transduced ex vivo to express the GBA and the one or more M2-promoting agents.

In some embodiments, the pluripotent cells are transduced with a viral vector selected from the group including an adeno-associated virus (AAV), an adenovirus, a parvovirus, a coronavirus, a rhabdovirus, a paramyxovirus, a picornavirus, an alphavirus, a herpes virus, a poxvirus, and a Retroviridae family virus.

In some embodiments, the viral vector is a Retroviridae family viral vector. In some embodiments, the Retroviridae family viral vector is a lentiviral vector. In some embodiments, the Retroviridae family viral vector is an alpharetroviral vector. In some embodiments, the Retroviridae family viral vector is a gammaretroviral vector. In some embodiments, the Retroviridae family viral vector includes a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

In some embodiments, the viral vector is an AAV selected from the group including AAV1, AAV2, AAV3, AAV4, AAV5, AAV6, AAV7, AAV8, AAV9, AAV10, and AAVrh74.

In some embodiments, the viral vector is a pseudotyped viral vector. In some embodiments, the viral vector is a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

In some embodiments, the pluripotent cells are transduced to express the GBA and the one or more M2-promoting agents from separate, monocistronic expression cassettes.

In some embodiments, the pluripotent cells are transduced to express the GBA and the one or more M2-promoting agents from a polycistronic expression cassette. In some embodiments, the pluripotent cells express a single M2-promoting agent, and the pluripotent cells are transduced to express the GBA and the M2-promoting agent from a bicistronic expression cassette. In some embodiments, the polycistronic expression cassette includes an internal ribosomal entry site (IRES) positioned between a polynucleotide encoding the GBA and a polynucleotide encoding one of the one or more M2-promoting agents. In some embodiments, the polycistronic expression cassette includes a foot-and-mouth disease virus 2A (FMDV 2A) polynucleotide positioned between a polynucleotide encoding the GBA and a polynucleotide encoding one of the one or more M2-promoting agents. In some embodiments, the pluripotent cells express GBA, a first M2-promoting agent, and a second M2-promoting agent from a single polycistronic expression cassette, wherein the polycistronic expression cassette includes a first FMDV 2A polynucleotide positioned between a polynucleotide encoding the GBA and a polynucleotide encoding the first M2-promoting agent, and wherein the polycistronic expression cassette further includes a second FMDV 2A polynucleotide positioned between the polynucleotide encoding the first M2-promoting agent and a polynucleotide encoding the second M2-promoting agent.

In some embodiments, the pluripotent cells are transfected ex vivo to express the GBA. In some embodiments, the pluripotent cells are transfected ex vivo to express the GBA and the one or more M2-promoting agents.

In some embodiments, the pluripotent cells are transfected using an agent selected from the group including a cationic polymer, diethylaminoethyl-dextran, polyethylenimine, a cationic lipid, a liposome, calcium phosphate, an activated dendrimer, and a magnetic bead; or a technique selected from the group including electroporation, Nucleofection, squeeze-poration, sonoporation, optical transfection, Magnetofection, and impalefection.

In some embodiments, the pluripotent cells are transfected ex vivo to express the GBA and the one or more M2-promoting agents from separate, monocistronic expression cassettes. In some embodiments, the monocistronic expression cassettes are located within two or more separate plasmids. In some embodiments, the monocistronic expression cassettes are located on a single plasmid.

In some embodiments, the pluripotent cells are transfected ex vivo to express the GBA and the one or more M2-promoting agents from a polycistronic expression cassette. In some embodiments, the pluripotent cells express a single M2-promoting agent, and the pluripotent cells are transfected ex vivo to express the GBA and the M2-promoting agent from a bicistronic expression cassette. In some embodiments, the polycistronic expression cassette includes an IRES positioned between a polynucleotide encoding the GBA and a polynucleotide encoding one of the one or more M2-promoting agents. In some embodiments, the polycistronic expression cassette includes an FMDV 2A polynucleotide positioned between a polynucleotide encoding the GBA and a polynucleotide encoding one of the one or more M2-promoting agents. In some embodiments, the pluripotent cells express GBA, a first M2-promoting agent, and a second M2-promoting agent from a single polycistronic expression cassette, wherein the polycistronic expression cassette includes a first FMDV 2A polynucleotide positioned between a polynucleotide encoding the GBA and a polynucleotide encoding the first M2-promoting agent, and wherein the polycistronic expression cassette further includes a second FMDV 2A polynucleotide positioned between the polynucleotide encoding the first M2-promoting agent and a polynucleotide encoding the second M2-promoting agent.

In some embodiments, expression of the GBA and/or the one or more M2-promoting agents in the pluripotent cells is driven using a ubiquitous promoter. Ubiquitous promoters include the elongation factor 1-alpha promoter and the phosphoglycerate kinase 1 promoter. In some embodiments, expression of the GBA and/or the one or more M2-promoting agents in the pluripotent cells is driven using a tissue-specific promoter. Tissue-specific promoters include CD68 molecule, C-X3-C motif chemokine receptor 1, integrin subunit alpha M, allograft inflammatory factor 1, purinergic receptor P2Y12, transmembrane protein 119, and colony stimulating factor 1 receptor.

In some embodiments, the transgene encoding GBA is operably linked to a polynucleotide encoding a protein destabilizing domain.

In some embodiments, the destabilizing domain is an FK506 binding protein 1A (FKBP12) destabilizing domain. In some embodiments, the FPBP12 destabilizing domain is an FKBP12 mutant selected from the group including F15S, V24A, H25R, E60G, L106P, M66T, R71G, D100G, D100N, E102G, and K105I. In some embodiments, the method includes administering Shield-1 to the subject in a quantity sufficient to induce expression of functional GBA.

In some embodiments, the destabilizing domain is an E. coli dihydrofolate reductase (ecDHFR) destabilizing domain. In some embodiments, the method includes administering trimethoprim to the subject in a quantity sufficient to induce expression of functional GBA.

In some embodiments, the destabilizing domain is a human estrogen receptor ligand binding domain (ERLBD) destabilizing domain. In some embodiments, the method includes administering CMP8 or 4-hydroxytamoxifen to the subject in a quantity sufficient to induce expression of functional GBA.

In some embodiments, one or more polynucleotides encoding each of the one or more M2-promoting agents in the pluripotent cells is operably linked to a protein destabilizing domain.

In some embodiments, the destabilizing domain is an FKBP12 destabilizing domain. In some embodiments, the FPBP12 destabilizing domain is an FKBP12 mutant selected from the group containing F15S, V24A, H25R, E60G, L106P, M66T, R71G, D100G, D100N, E102G, and K105I. In some embodiments, the method includes administering Shield-1 to the subject in a quantity sufficient to induce expression of a functional M2-promoting agent.

In some embodiments, the destabilizing domain is an ecDHFR destabilizing domain. In some embodiments, the method includes administering trimethoprim to the subject in a quantity sufficient to induce expression of a functional M2-promoting agent.

In some embodiments, the destabilizing domain is a human ERLBD destabilizing domain. In some embodiments, the method includes administering CMP8 or 4-hydroxytamoxifen to the subject in a quantity sufficient to induce expression of a functional M2-promoting agent.

In some embodiments, the composition is administered to the subject in an amount sufficient to increase the quantity of M2 microglia in the brain of the subject relative to the quantity of M1 microglia in the brain of the subject, decrease the level of one or more pro-inflammatory cytokines in the brain of the subject, increase the level of one or more anti-inflammatory cytokines in the brain of the subject, improve the cognitive performance of the subject, improve the motor function of the subject, reduce dopaminergic neuron loss in the subject, and/or reduce α-synuclein levels or aggregation thereof in the subject.

In some embodiments, the subject is a human.

In another aspect, the invention provides a composition containing a population of pluripotent cells that express (i) a first transgene encoding non-secreted GBA; and (ii) one or more transgenes that each encode an M2-promoting agent.

In another aspect, the invention provides a composition containing a population of pluripotent cells that express (i) a first transgene encoding secreted GBA; and (ii) one or more transgenes that each encode an M2-promoting agent.

In some embodiments of any of the preceding two aspects, the pluripotent cells are CD34+ cells. In some embodiments, the CD34+ cells are embryonic stem cells. In some embodiments, the CD34+ cells are induced pluripotent stem cells. In some embodiments, the CD34+ cells are hematopoietic stem cells. In some embodiments, the CD34+ cells are myeloid progenitor cells. In some embodiments, at least one of the one or more transgenes that each encode an M2-promoting agent encodes a cytokine selected from the group consisting of IL-25, IL-4, IL-10, IL-13, and TGF-β. In some embodiments, at least one of the one or more transgenes that each encode an M2-promoting agent encodes IL-25. In some embodiments, at least one of the one or more transgenes that each encode an M2-promoting agent encodes an agent selected from the group consisting of a glucocorticoid receptor, a PPAR, PPARγ, PPARβ/δ, an estrogen receptor, NR4A2, KDM6B, MSX3, FAM19A3, NF-κB p50, miR124, miR21, and miR181c, CX3CL1, CX3CR1, CD200, CD200R, CFH, CD47, CD55, CD46, ADGRE5, SIRPα, and siglecs. In some embodiments, the pluripotent cells are transduced ex vivo to express the GBA and the one or more M2-promoting agents. In some embodiments, the pluripotent cells are transfected ex vivo to express the GBA and the one or more M2-promoting agents. In some embodiments, endogenous GBA is disrupted in the pluripotent cells.

In an additional aspect, the invention provides kits containing compositions according to any of the above aspects and embodiments and a package insert. In some embodiments, the package insert instructs a user of the kit to perform a method according to any of the above aspects and embodiments.

### BRIEF DESCRIPTION OF THE DRAWINGS

FIG. 1A is a diagram of an exemplary GBA transgene construct containing, in the N-terminal to C-terminal direction, a polypeptide sequence encoding a GBA signal peptide (SP) and a GBA catalytic domain that has been codon optimized for expression in human cells (GBA-co CD).

FIG. 1B is a diagram of an exemplary GBA transgene construct containing, in the N-terminal to C-terminal direction, a polypeptide sequence encoding a GBA SP, an Rb domain of ApoE (Rb-ApoE), and codon-optimized GBA (GBA-co).

FIG. 1C is a diagram of an exemplary GBA transgene construct containing, in the N-terminal to C-terminal direction, a polypeptide sequence encoding a codon-optimized human insulin-like growth factor II secretory signal peptide (IGF-II-co SSP), a GILT-tag (GILT) containing an R37A mutation, a linker (L), and GBA-co.

FIG. 1D is a diagram of an exemplary GBA transgene construct containing, in the N-terminal to C-terminal direction, a polypeptide sequence encoding an IGF-II-co SSP, a first linker (L), Rb-ApoE, a second linker, GILT containing an R37A mutation, a third linker, and GBA-co.

FIG. 1E is a diagram of an exemplary GBA transgene construct containing, in the N-terminal to C-terminal direction, a polypeptide sequence encoding an IGF-II-co SSP, GILT containing an R37A mutation, a linker, Rb-ApoE, a linker, and GBA-co containing a micro RNA targeting sequence (miRT) in the three prime untranslated region (3′-UTR).

FIG. 1F is a diagram of an exemplary GBA transgene construct containing, in the N-terminal to C-terminal direction, a polypeptide sequence encoding an IGF-II-co SSP, a first linker (L), GBA-co, a second linker, and GILT containing a R37A mutation.

FIG. 1G is a diagram of an exemplary GBA transgene construct containing, in the N-terminal to C-terminal direction, a polypeptide sequence encoding an IGF-II-co SSP, a first linker (L), GBA-co, a second linker, and Rb-ApoE.

FIGS. 2A-2D are a series of bar plots demonstrating GBA enzymatic activity and protein levels in mammalian cell lines transduced with either green fluorescent protein (GFP) or GBA constructs. FIG. 2A and FIG. 2C show GBA enzymatic activity measured from cell lysates of human HEK293T cells and mouse RAW264.7 cells, respectively, following lentiviral transduction with GFP (black bars) or codon-optimized GBA (GBAco) constructs (grey bars). GBA enzymatic activity was measured in using a 4-methylumbelliferyl β-D-glucopyranoside (4MUG) substrate, which is enzymatically converted by GBA to produce a fluorescent product, 4-Methylumbelliferone (4MU). Tested GBAco constructs included: 1) GBAco alone; 2) GBAco, a C-terminal glycosylation independent lysosomal targeting (GILT) tag, and a peptide linker; 3) GBAco and a modified signal peptide sequence; 4) GBAco, a GILT tag, and a rigid peptide linker; or 5) GBAco, a GILT tag, and an XTEN linker. All lentivirally-encoded GBAco constructs resulted in increased GBA enzymatic activity in both of the tested cell lines. FIG. 2B and FIG. 2D show GBA protein levels as assayed by Western blot analysis of cell lysates from HEK293T and RAW264.7 cells, respectively, following lentiviral transduction with GFP (black bars) or codon-optimized GBA (GBAco) constructs (grey bars). All lentivirally-encoded GBAco constructs resulted in increased GBA protein levels in both of the tested cell lines. Data are represented as mean±SEM; n=3 independent transductions. Asterisks signify p-values less than 0.05, as determined using one-way ANOVA tests for statistical significance.

FIG. 3 is a Western blot analysis of glycosylated, transgene-derived GBA taken from cell lysates of HEK293T cells. Cells were transduced with one of three lentivirally-encoded GBAco constructs selected from the group including: 1) GBAco alone; 2) GBAco, a GILT tag, and a rigid peptide linker; or 3) GBAco, a GILT tag, and an XTEN linker. Cell lysates were treated with either EndoH or PNGase F glycosidases to assess changes in N-linked glycosylation through increased electrophoretic mobility. Western blot analysis detected engineered GBAco proteins at the predicted molecular weights, suggesting that GILT and linker peptides were stably expressed. Furthermore, electrophoretic mobility of engineered GBA proteins increased after enzymatic de-glycosylation, demonstrating that these proteins undergo physiological glycosylation.

FIGS. 4A-4B are a series of bar plots demonstrating GBA enzymatic activity in mouse lineage-negative (Lin⁻) cells from wildtype and GBA mutant mice. Lin⁻ cells were isolated from the bone marrow and transduced with lentiviral vectors encoding GFP or GBAco. FIG. 4A shows a bar plot of GBA enzymatic activity in Lin⁻ cell lysate from wildtype and GBA-deficient transgenic mice (Gba^{D409V/+}, Gba^{D409V/+}; Thy1-SNCA and Gba^{D409V/D409V}) transduced with a vector encoding GFP (black bars) or GBAco (grey bars). Enzymatic assays of Lin⁻ cells demonstrated that the heterozygous and homozygous Gba mutations reduced GBA activity by 43% and 92%, respectively, in the absence of GBA transgenes (WT: 13.04±0.644 nmol hr⁻¹ mg⁻¹; Gba^{D409V/+}; 7.49±0.293 nmol hr⁻¹ mg⁻¹; Thy1-SNCA; Gba^{D409V/+}: 7.14±0.252 nmol hr⁻¹ mg⁻¹; Thy1-SNCA; Gba^{D409V/D409V}: 1.20±0.114 nmol hr⁻¹ mg⁻¹; p<0.001, ANOVA with Tukey post-hoc analysis, FIG. 4A). Lentiviral transduction of Lin⁻ cells with a GBAco construct substantially increased GBA activity across all tested mouse lines (compared to GFP control; p<0.001, ANOVA with Tukey post-hoc analysis). FIG. 4B shows a bar plot of GBA activity in Lin⁻ cell conditioned medium from Gba^{D409V/+}, Gba^{D409V/+}; Thy1-SNCA and Gba^{D409V/D409V} mice (total n=8 animals) after lentiviral transduction with GFP (black bars) or GBAco (grey bars), demonstrating increased detection of GBA activity. Data are represented as mean±SEM (n=1 independent transduction). Combined, these findings demonstrate that lentiviral GBAco constructs produce a functional GBA enzyme in hematopoietic stem cells (e.g., mouse Lin⁻ cells) and can rescue GBA activity and expression levels in mouse models of GBA deficiency.

### DEFINITIONS

As used herein, “administration” refers to providing or giving a subject a therapeutic agent (e.g., pluripotent cells, such as CD34+ cells, that express a transgene encoding GBA or pluripotent cells that express a transgene encoding GBA. For example, the pluripotent cells that express a transgene encoding GBA may additionally express one or more transgenes that each encode an M2-promoting agent), by any effective route. Exemplary routes of administration are described herein below (e.g., intracerebroventricular (ICV) injection, intravenous (IV) injection, and stereotactic injection).

As used herein, “allogeneic” means cells, tissue, DNA, or factors taken or derived from a different subject of the same species. For example, in the context where allogeneic transduced pluripotent cells are administered to a subject with Parkinson's disease, pluripotent cells derived from cells obtained from a patient that is not the subject are transduced or transfected with a vector that directs the expression of GBA and, optionally, one or more M2-promoting agents and the transduced cells are administered to the subject. The phrase “directs expression” refers to the polynucleotide containing a sequence that encodes the molecule to be expressed. The polynucleotide may contain additional sequence that enhances expression of the molecule in question.

As used herein, “autologous” refers to cells, tissue, DNA, or factors taken or derived from an individual's own tissues, cells, or DNA. For example, in the context where autologous transduced pluripotent cells are administered to a subject with Parkinson's disease, pluripotent cells derived from cells obtained from the subject are transduced or transfected with a vector that directs the expression of GBA and, optionally, one or more M2-promoting agents and the transduced cells are administered to the subject.

As used herein, the term “ApoE” refers to apolipoprotein E, a member of a class of proteins involved in lipid transport. Apolipoprotein E is a fat-binding protein (apolipoprotein) that is part of the chylomicron and intermediate-density lipoprotein (IDLs). These are essential for the normal processing (catabolism) of triglyceride-rich lipoproteins. ApoE is encoded by the APOE gene. The term “ApoE” also refers to variants of the wild-type ApoE protein, such as proteins having at least 85% identity (e.g., 85%, 86% e.g., 85%, 86%, 87%, 88%, 89%, 90%, 91%, 92%, 93%, 94%, 95%, 96%, 97%, 98%, 99%, or 99.9% identity, or more) to the amino acid sequence of wild-type ApoE, which is set forth in SEQ ID NO. 21.

As used herein, the term “cell type” refers to a group of cells sharing a phenotype that is statistically separable based on gene expression data. For example, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, “codon optimization” refers a process of modifying a nucleic acid sequence in accordance with the principle that the frequency of occurrence of synonymous codons (e.g., codons that code for the same amino acid) in coding DNA is biased in different species. Such codon degeneracy allows an identical polypeptide to be encoded by a variety of nucleotide sequences. Sequences modified in this way are referred to herein as “codon-optimized.” This process may be performed on any of the sequences described in this specification to enhance expression or stability. Codon optimization may be performed in a manner such as that described in, e.g., U.S. Pat. Nos. 7,561,972, 7,561,973, and 7,888,112, each of which is incorporated herein by reference in its entirety. The sequence surrounding the translational start site can be converted to a consensus Kozak sequence according to known methods. See, e.g., Kozak et al, Nucleic Acids Res. 15 (20): 8125-8148, incorporated herein by reference in its entirety. Multiple stop codons can be incorporated.

As used herein, the terms “condition” and “conditioning” refer to processes by which a subject is prepared for receipt of a transplant containing pluripotent stem cells (e.g., CD34+ cells). Such procedures promote the engraftment of a pluripotent stem cell transplant, for example, by selectively depleting endogenous microglia or hematopoietic stem cells, thereby creating a vacancy filled by an exogenous pluripotent stem cell transplant. According to the methods described herein, a subject may be conditioned for pluripotent stem cell transplant therapy by administration to the subject of one or more agents capable of ablating endogenous microglia and/or hematopoietic stem or progenitor cells (e.g., busulfan, treosulfan, PLX3397, PLX647, PLX5622, and clodronate liposomes), radiation therapy, or a combination thereof. Conditioning may be myeloablative or non-myeloablative. Other cell-ablating agents and methods well known in the art (e.g., antibody-drug conjugates) may also be used.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in table 1 below.

TABLE 1
Representative physicochemical properties
of naturally-occurring amino acids

| | | | | Electrostatic | |
|---|---|---|---|---|---|
| | 3 | 1 | Side- | character at | |
| | Letter | Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non- | neutral | intermediate |
| | | | polar | | |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the terms “destabilizing domain” and “destabilization domain” refer to regulatory elements capable of destabilizing the proteins to which they are operably linked. These domains can be stabilized by specific exogenous ligands, such as small molecules. Exemplary destabilizing domains include mutants of the human FK506- and rapamycin-binding protein (FKBP12), which confer instability to other proteins fused to these destabilizing domains. FKBP12 mutants include N-terminal mutants F15S, V24A, H25R, E60G, and L106P, and C-terminal mutants M66T, R71G, D100G, D100N, E102G, and K105I, as characterized in Banaszynski et al., Cell 126:995 (2006), the disclosure of which is incorporated herein by reference as it pertains to FKBP12 destabilizing domains. FKBP12 destabilizing domains promote protein degradation. The small molecule ligand Shield-1 (Shld1) can be used to stabilize FKBP12 mutant-containing proteins by protecting them from degradation. Other destabilizing domains that can be used to regulate expression of GBA or M2-promoting agents include mutants of the E. coli dihydrofolate reductase (ecDHFR) and mutants of the human estrogen receptor ligand binding domain (ERLBD), which confer instability resulting in degradation when fused to a protein of interest and can be stabilized by small molecule ligand trimethoprim (TMP), or by CMP8 or 4-hydroxytamoxifen (4OHT), respectively, as described in Iwamoto et al., Chem Biol. 17:981 (2010) and Miyazaki et al., J Am Chem Soc., 134:3942 (2012), the disclosures of each of which are incorporated herein by reference as they pertain to destabilization domain systems.

As used herein, the term “disrupt”, with respect to a gene, refers to preventing the formation of a functional gene product. A gene product is functional if it fulfills its normal (wild-type) functions. Disruption of the gene prevents expression of a functional factor encoded by the gene and contains an insertion, deletion, or substitution of one or more bases in a sequence encoded by the gene and/or a promoter and/or an operator that is necessary for expression of the gene in the animal. The disrupted gene may be disrupted by, e.g., removal of at least a portion of the gene from a genome of the animal, alteration of the gene to prevent expression of a functional factor encoded by the gene, an interfering RNA, or expression of a dominant negative factor by an exogenous gene. Materials and methods for genetically modifying pluripotent stem/progenitor cells (e.g., CD34+ cells) so as to disrupt the expression of one or more genes are detailed in U.S. Pat. No. 8,518,701; US 2010/0251395; and US 2012/0222143, the disclosures of each of which are incorporated herein by reference in their entirety (in case of conflict, the instant specification is controlling).

As used herein, the terms “effective amount,” “therapeutically effective amount,” and a “sufficient amount” of composition, vector construct, viral vector, or cell described herein refer to a quantity sufficient to, when administered to the subject, including a mammal, for example a human, effect beneficial or desired results, including clinical results, and, as such, an “effective amount” or synonym thereto depends upon the context in which it is being applied. For example, in the context of treating Parkinson's disease, it is an amount of the composition, vector construct, viral vector, or cell sufficient to achieve a treatment response as compared to the response obtained without administration of the composition, vector construct, viral vector or cell. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition, vector construct, viral vector, or cell of the present disclosure is an amount which results in a beneficial or desired result in a subject as compared to a control. As defined herein, a therapeutically effective amount of a composition, vector construct, viral vector, or cell of the present disclosure may be readily determined by one of ordinary skill by routine methods known in the art. Dosage regime may be adjusted to provide the optimum therapeutic response.

As used herein, the terms “embryonic stem cell” and “ES cell” refer to an embryo-derived totipotent or pluripotent stem cell, derived from the inner cell mass of a blastocyst that can be maintained in an in vitro culture under suitable conditions. ES cells are capable of differentiating into cells of any of the three vertebrate germ layers, e.g., the endoderm, the ectoderm, or the mesoderm. ES cells are also characterized by their ability propagate indefinitely under suitable in vitro culture conditions. See, for example, Thomson et al., Science 282:1145 (1998).

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. Expression of a gene of interest in a subject can manifest, for example, by detecting: an increase in the quantity or concentration of mRNA encoding a corresponding protein (as assessed, e.g., using RNA detection procedures described herein or known in the art, such as quantitative polymerase chain reaction (qPCR) and RNA seq techniques), an increase in the quantity or concentration of a corresponding protein (as assessed, e.g., using protein detection methods described herein or known in the art, such as enzyme-linked immunosorbent assays (ELISA), among others), and/or an increase in the activity of a corresponding protein (e.g., in the case of an enzyme, as assessed using an enzymatic activity assay described herein or known in the art) in a sample obtained from the subject.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “FMDV 2A” refers to the 2A nucleic acid sequence of the food-and-mouth disease virus. In general, an FMDV 2A sequence is a feature that allows the coordinated expression of multiple proteins in equimolar amounts from a single open reading frame. FMDV 2A mediates a cotranslational cleavage event, which separates proteins linked by 2A sequences. Multiple 2A sequences may be used in one vector, and coexpression of proteins linked by 2A will work in most eukaryotic cells as cleavage activity depends on eukaryotic ribosomes. For an example of the use of FMDV 2A to express multiple proteins, see Ryan and Drew, EMBO Journal 13:928 (1994), the disclosure of which is incorporated herein by reference.

As used herein, the term “functional potential” as it pertains to a stem cell, such as a hematopoietic stem cell, refers to the functional properties of stem cells which include 1) multi-potency (which refers to the ability to differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal (which refers to the ability of stem cells to give rise to daughter cells that have equivalent potential as the mother cell, and further that this ability can repeatedly occur throughout the lifetime of an individual without exhaustion), and 3) the ability of stem cells or progeny thereof to be reintroduced into a transplant recipient whereupon they home to the stem cell niche and re-establish productive and sustained cell growth and differentiation.

As used herein, the term “furin-resistant IGF-II mutein” refers to an insulin-like growth factor II (IGF-II)-based peptide containing an altered amino acid sequence relative to wild-type IGF-II (SEQ ID NO. 12) that abolishes at least one native furin protease cleavage site or changes a sequence close or adjacent to a native furin protease cleavage site such that the furin cleavage is prevented, inhibited, reduced, or slowed down as compared to a wild-type human IGF-II peptide. As used herein, a furin-resistant IGF-II mutein is also referred to as an IGF-II mutein that is resistant to furin. Exemplary furin-resistant IGF-II muteins contain amino acid substitutions at positions corresponding to Arg37 and/or Arg40 of SEQ ID NO. 12.

As used herein, the term “furin protease cleavage site” (also referred to as “furin cleavage site” or “furin cleavage sequence”) refers to the amino acid sequence of a peptide or protein that serves as a recognition sequence for enzymatic protease cleavage by furin or furin-like proteases. Typically, a furin protease cleavage site has a consensus sequence Arg-X-X-Arg, where X is any amino acid. The cleavage site is positioned after the carboxy-terminal arginine (Arg) residue in the sequence. In some embodiments, a furin cleavage site has a consensus sequence Lys/Arg-X-X-X-Lys/Arg-Arg, where X is any amino acid. The cleavage site is positioned after the carboxy-terminal arginine (Arg) residue in the sequence.

As used herein, the term “furin” refers to any protease that can recognize and cleave the furin protease cleavage site as defined herein, including furin or furin-like protease. Furin is also known as paired basic amino acid cleaving enzyme (PACE). Furin belongs to the subtilisin-like proprotein convertase family. The gene encoding furin is known as FUR (FES Upstream Region).

As used herein, the term “glycosylation independent lysosomal targeting” or “GILT” refers to lysosomal targeting that is mannose-6-phosphate (M6P)-independent. A GILT tag may be used to target a protein (e.g., GBA) expressed as a GILT-tagged fusion protein (e.g., a GBA fusion protein coupled to an IGF-II mutein), to the lysosome.

As used interchangeably herein, the terms “cation-independent mannose-6-phosphate receptor (CI-MPR),” “M6P/IGF-II receptor,” “CI-MPR/IGF-II receptor,” “IGF-II receptor” or “IGF2 Receptor,” or abbreviations thereof, refer to the cellular receptor which binds both M6P and IGF-II.

As used herein, patients suffering from “GBA-associated Parkinson's disease” or “GBA-associated PD” are those patients that have been diagnosed as having Parkinson's disease and also contain a deleterious mutation in the GBA gene. Severely pathogenic mutations include c.84GGIns, IVS2+1 G>A, p.V394L, p.D409H, p.L444P and RecTL, which are linked to a 9.92 to 21.29 odds-ratio of developing PD. Mild GBA mutations p.N370S and p.R496H are linked to an odds-ratio of 2.84-4.94 of developing PD. The mutation p.E326K has also been identified as a PD risk factor. GBA mutations are discussed in Barkhuizen et al., Neurochemistry International 93:6 (2016) and Sidransky and Lopez, Lancet Neurol. 11:986 (2012), the disclosures of which are incorporated herein by reference as they pertain to human GBA mutations.

As used herein, the terms “glucocerebrosidase” and “GBA” refer to the lysosomal enzyme responsible for the metabolism of glucocerebroside (also known as glucosylceramide) to glucose and ceramide. The gene is located on chromosome 1q21 and is also known as GBA1. The terms “glucocerebrosidase” and “GBA” also refer to variants of wild-type glucocerebrosidase enzymes and nucleic acids encoding the same, such as variant proteins having at least 85% sequence identity (e.g., 85%, 86%, 87%, 88%, 89%, 90%, 91%, 92%, 93%, 94%, 95%, 96%, 97%, 98%, 99%, or 99.9% identity, or more) to the amino acid sequence of a wild-type GBA enzyme (e.g., SEQ ID NO. 1) or polynucleotides having at least 85% sequence identity (e.g., 85%, 86%, 87%, 88%, 89%, 90%, 91%, 92%, 93%, 94%, 95%, 96%, 97%, 98%, 99%, or 99.9% identity, or more) to the nucleic acid sequence of a wild-type GBA gene (e.g., SEQ ID NO. 6), provided that the GBA analog encoded retains the therapeutic function of wild-type GBA. “GBA” may also refer to a GBA protein in which the natural signal peptide is present. The terms “glucocerebrosidase” and “GBA” may also refer to codon-optimized polynucleotides encoding GBA, such as codon-optimized polynucleotides having at least 85% sequence identity (e.g., 85%, 86%, 87%, 88%, 89%, 90%, 91%, 92%, 93%, 94%, 95%, 96%, 97%, 98%, 99%, or 99.9% identity, or more) to the nucleic acid sequence of SEQ ID NO. 3. Alternatively, “GBA” may refer to a GBA protein in which the natural signal peptide has been removed (e.g., the mature protein). GBA may also refer to the catalytic domain of GBA, such as a domain containing residues 76-381 and 416-430 of SEQ ID NO. 1, or a variant having at least 85% sequence identity (e.g., 85%, 86%, 87%, 88%, 89%, 90%, 91%, 92%, 93%, 94%, 95%, 96%, 97%, 98%, 99%, or greater, sequence identity) to such a domain. As used herein, GBA may refer to the lysosomal enzyme or the gene encoding this protein, depending upon the context, as will be appreciated by one of skill in the art.

As used herein, the terms “hematopoietic stem cells” and “HSCs” refer to immature blood cells having the capacity to self-renew and to differentiate into mature blood cells of diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). It is known in the art that such cells may or may not include CD34+ cells. CD34+ cells are immature cells that express the CD34 cell surface marker. In humans, CD34+ cells are believed to include a subpopulation of cells with the stem cell properties defined above, whereas in mice, HSCs are CD34-. In addition, HSCs also refer to long term repopulating HSC (LT-HSC) and short-term repopulating HSC (ST-HSC). LT-HSC and ST-HSC are differentiated, based on functional potential and on cell surface marker expression. For example, human HSC are a CD34+, CD38−, CD45RA−, CD90+, CD49F+, and lin−(negative for mature lineage markers including CD2, CD3, CD4, CD7, CD8, CD10, CD11B, CD19, CD20, CD56, CD235A). In mice, bone marrow LT-HSC are CD34−, SCA-1+, C-kit+, CD135−, Slamf1/CD150+, CD48−, and lin−(negative for mature lineage markers including Ter119, CD11b, Gr1, CD3, CD4, CD8, B220, IL-7ra), whereas ST-HS Care CD34+, SCA-1+, C-kit+, CD135-, Slamf1/CD150+, and lin−(negative for mature lineage markers including Ter119, CD11b, Gr1, CD3, CD4, CD8, B220, IL-7ra). In addition, ST-HSC are less quiescent (i.e., more active) and more proliferative than LT-HSC under homeostatic conditions. However, LT-HSC have greater self-renewal potential (i.e., they survive throughout adulthood, and can be serially transplanted through successive recipients), whereas ST-HSC have limited self-renewal (i.e., they survive for only a limited period of time, and do not possess serial transplantation potential). Any of these HSCs can be used in any of the methods described herein. Optionally, ST-HSCs are useful because they are highly proliferative and thus, can more quickly give rise to differentiated progeny.

As used herein, the term “HLA-matched” refers to a donor-recipient pair in which none of the HLA antigens are mismatched between the donor and recipient, such as a donor providing a hematopoietic stem cell graft to a recipient in need of hematopoietic stem cell transplant therapy. HLA-matched (i.e., where all of the 6 alleles are matched) donor-recipient pairs have a decreased risk of graft rejection, as endogenous T cells and NK cells are less likely to recognize the incoming graft as foreign, and are thus less likely to mount an immune response against the transplant.

As used herein, the term “HLA-mismatched” refers to a donor-recipient pair in which at least one HLA antigen, in particular with respect to HLA-A, HLA-B, HLA-C, and HLA-DR, is mismatched between the donor and recipient, such as a donor providing a hematopoietic stem cell graft to a recipient in need of hematopoietic stem cell transplant therapy. In some embodiments, one haplotype is matched and the other is mismatched. HLA-mismatched donor-recipient pairs may have an increased risk of graft rejection relative to HLA-matched donor-recipient pairs, as endogenous T cells and NK cells are more likely to recognize the incoming graft as foreign in the case of an HLA-mismatched donor-recipient pair, and such T cells and NK cells are thus more likely to mount an immune response against the transplant.

As used herein, the terms “induced pluripotent stem cell,” “iPS cell,” and “iPSC” refer to a pluripotent stem cell that can be derived directly from a differentiated somatic cell. Human iPS cells can be generated by introducing specific sets of reprogramming factors into a non-pluripotent cell that can include, for example, Oct3/4, Sox family transcription factors (e.g., Sox1, Sox2, Sox3, Soxl5), Myc family transcription factors (e.g., c-Myc, 1-Myc, n-Myc), Kruppel-like family (KLF) transcription factors (e.g., KLF1, KLF2, KLF4, KLF5), and/or related transcription factors, such as NANOG, LIN28, and/or Glis1. Human iPS cells can also be generated, for example, by the use of miRNAs, small molecules that mimic the actions of transcription factors, or lineage specifiers. Human iPS cells are characterized by their ability to differentiate into any cell of the three vertebrate germ layers, e.g., the endoderm, the ectoderm, or the mesoderm. Human iPS cells are also characterized by their ability propagate indefinitely under suitable in vitro culture conditions. See, for example, Takahashi and Yamanaka, Cell 126:663 (2006).

As used herein, the term “IRES” refers to an internal ribosomal entry site. In general, an IRES sequence is a feature that allows eukaryotic ribosomes to bind an mRNA transcript and begin translation without binding to a 5′ capped end. An mRNA containing an IRES sequence produces two translation products, one initiating form the 5′ end of the mRNA and the other from an internal translation mechanism mediated by the IRES.

As used herein, the term “M2-promoting agent” refers to an agent that affects the polarization of macrophages or microglia by shifting cells toward a more anti-inflammatory state. M2-promoting agents include those that can reduce secretion and/or expression of pro-inflammatory cytokines and genes associated with classical activation/M1 by macrophages or microglia, e.g., IL-1β, tumor necrosis factor (TNF), iNOS, or IL-12. For example, M2-promoting agents are substances capable of reducing secretion or expression of pro-inflammatory cytokines and genes associated with classical M1 activation, such as IL-1β, TNF, iNOS, or IL-12 by 10%, 15%, 20%, 25%, 30%, 35%, 40%, 45%, 50%, 55%, 60%, 65%, 70%, 75%, 80%, 85%, 90%, 95%, 100%, 200%, 500%, or more, when a population of activated macrophages or microglia is cultured in the presence of the substance, for example, for a period of from about 2 hours to 24 hours (e.g., for a period of about 2 hours, 4 hours, 6 hours, 8 hours, 10 hours, 12 hours, 14 hours, 16 hours, 18 hours, 20 hours, 22 hours, or 24 hours). M2-promoting agents additionally include those capable of increasing the secretion and/or expression of anti-inflammatory cytokines and genes associated with alternative activation/M2 by macrophages or microglia, e.g., IL-25, IL-10, transforming growth factor beta (TGF-β), CD163, or TREM2, or altering cell morphology or shape, e.g., increasing ramification. For example, M2-promoting agents are substances capable of increasing secretion or expression of anti-inflammatory cytokines and genes associated with alternative M2 activation, such as IL-10, TGF-β, CD163, or TREM2 by 10%, 15%, 20%, 25%, 30%, 35%, 40%, 45%, 50%, 55%, 60%, 65%, 70%, 75%, 80%, 85%, 90%, 95%, 100%, 200%, 500%, or more, when a population of activated macrophages or microglia is cultured in the presence of the substance, for example, for a period of from about 2 hours to 24 hours (e.g., for a period of about 2 hours, 4 hours, 6 hours, 8 hours, 10 hours, 12 hours, 14 hours, 16 hours, 18 hours, 20 hours, 22 hours, or 24 hours). Techniques suitable for determining the concentration of a secreted cytokine in a cell culture medium are, for example, enzyme-linked immunosorbent assays (ELISA) known in the art. Additionally, techniques that can be used to quantify the expression of a gene of interest include, without limitation, quantitative reverse-transcriptase polymerase chain reaction (RT-PCR) assays known in the art.

As used herein, the term “monocistronic” refers to an RNA or DNA construct that contains the coding sequence for a single protein or polypeptide product.

As used herein, the term “myeloablative” or “myeloablation” refers to a conditioning regiment that substantially impairs or destroys the hematopoietic system, typically by exposure to a cytotoxic agent (e.g., busulfan) or radiation. Myeloablation encompasses complete myeloablation brought on by high doses of cytotoxic agent or total body irradiation that destroys the hematopoietic system.

As used herein, the term “non-myeloablative” or “myelosuppressive” refers to a conditioning regiment that does not eliminate substantially all hematopoietic cells of host origin.

As used herein, the term “polycistronic” refers to an RNA or DNA construct that contains the coding sequence for more than one protein or polypeptide product. Exemplary polycistronic vectors include those described in WO 1993/003143, Ryan and Drew, EMBO Journal 13:928 (1994), Szymczak and Vignali, Expert Opin Biol Ther. 5:627 (2005), Szymczak et al., Nat Biotechnol. 22:589 (2004), and Osborn et al., Molecular Therapy 12:569 (2005).

As used herein, the term “pluripotent cell” refers to a cell that possesses the ability to develop into more than one differentiated cell type, such as a cell type of the hematopoietic lineage (e.g., granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Examples of pluripotent cells are embryonic stem cells (ES cells) and induced pluripotent stem cells (iPS cells or iPSCs). Exemplary pluripotent cells are CD34+ cells.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the term “promoter” refers to a recognition site on DNA that is bound by an RNA polymerase. The polymerase drives transcription of the transgene. Exemplary promoters suitable for use with the compositions and methods described herein are described, for example, in Sandelin et al., Nature Reviews Genetics 8:424 (2007), the disclosure of which is incorporated herein by reference as it pertains to nucleic acid regulatory elements.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:
100 multiplied by (the fraction X/Y)
   where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, a potent “receptor-binding peptide (Rb) derived from ApoE”, has the ability to translocate proteins across the BBB into the brain when engineered as fusion proteins. This method can therefore function to selectively open the BBB for therapeutic agents (e.g., soluble GBA) when engineered as a fusion protein. This peptide can be readily attached to diagnostic or therapeutic agents without jeopardizing their biological functions or interfering with the important biological functions of ApoE due to the utilization of the Rb domain of ApoE, rather than the entire ApoE protein. This pathway is also an alternative uptake pathway that can facilitate further/secondary distribution within the brain after the agents reach the CNS due to the widespread expression of LDLRf members in brain parenchyma. Exemplary Rb domains can be found in the N-terminus of ApoE. For example, Rb domains useful in conjunction with the compositions and methods described herein are polypeptides having the amino acid sequence of residues 1 to 191 of SEQ ID NO. 21, residues 25 to 185 of SEQ ID NO. 21, residues 50 to 180 of SEQ ID NO. 21, residues 75 to 175 of SEQ ID NO. 21, residues 100 to 170 of SEQ ID NO. 21, or residues 125 to 165 of SEQ ID NO. 21, as well as variants thereof, such as polypeptides having at least 85% sequence identity (e.g., 85%, 86%, 87%, 88%, 89%, 90%, 91%, 92%, 93%, 94%, 95%, 96%, 97%, 98%, 99%, or greater, sequence identity) with respect to any of these sequences. An exemplary Rb domain is the region of ApoE having the amino acid sequence of residues 159 to 167 of SEQ ID NO. 21.

As used herein, the term “regulatory sequence” includes promoters, enhancers and other expression control elements (e.g., polyadenylation signals) that control the transcription or translation of the antibody chain genes. Such regulatory sequences are described, for example, in Goeddel, Gene Expression Technology: Methods in Enzymology 185 (Academic Press, San Diego, Calif., (1990)); incorporated herein by reference.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject.

As used herein, the term “secretory signal peptide” refers to a short (usually between 16-60 amino acids) peptide region within the precursor protein that directs secretion of the precursor protein from the cytoplasm of the host into the periplasmic space or into the extracellular space. Such secretory signal peptides are generally located at the amino terminus of the precursor protein. In some embodiments, the secretory signal peptide is linked to the amino terminus and may be heterologous to the protein to which it is linked. Typically, secretory signal peptides are cleaved during transit through the cellular secretion pathway. Cleavage is not essential as long as the secreted protein retains its desired activity. Exemplary secretory signal peptides include those from IGF-II, alpha-1 antitrypsin, and GBA.

As used herein, the term “signal peptide” refers to a short (usually between 16-60 amino acids) polypeptide present on precursor proteins (typically at the N terminus), which is typically absent from the mature protein. The signal peptide directs the transport of the translated protein through the cell membrane. Signal peptides may also be called targeting signals, transit peptides, localization signals, or signal sequences. For example, the signal sequence may be a co-translational or post-translational signal peptide. Exemplary signal peptides include the GBA signal peptide (e.g., a 39-amino acid GBA signal peptide as described in Sorge et al., Am. J. Hum. Genet. 41:1016-1024 (1987); incorporated herein by reference).

As used herein, the terms “stem cell” and “undifferentiated cell” refer to a cell in an undifferentiated or partially differentiated state that has the developmental potential to differentiate into multiple cell types. A stem cell is capable of proliferation and giving rise to more such stem cells while maintaining its functional potential. Stem cells can divide asymmetrically, which is known as obligatory asymmetrical differentiation, with one daughter cell retaining the functional potential of the parent stem cell and the other daughter cell expressing some distinct other specific function, phenotype and/or developmental potential from the parent cell. The daughter cells themselves can be induced to proliferate and produce progeny that subsequently differentiate into one or more mature cell types, while also retaining one or more cells with parental developmental potential. A differentiated cell may derive from a multipotent cell, which itself is derived from a multipotent cell, and so on. Alternatively, some of the stem cells in a population can divide symmetrically into two stem cells. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. In some embodiments, the term stem cell refers generally to a naturally occurring parent cell whose descendants (progeny cells) specialize, often in different directions, by differentiation, e.g., by acquiring completely individual characters, as occurs in progressive diversification of embryonic cells and tissues. Some differentiated cells also have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype, a term often referred to as “dedifferentiation” or “reprogramming” or “retrodifferentiation” by persons of ordinary skill in the art.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, DEAE-dextran transfection, Nucleofection, squeeze-poration, sonoporation, optical transfection, Magnetofection, impalefection, and the like.

As used herein, the term “transgene” refers to a recombinant nucleic acid (e.g., DNA or cDNA) encoding a gene product (e.g., GBA). The gene product may be an RNA, peptide, or protein. In addition to the coding region for the gene product, the transgene may include or be operably linked to one or more elements to facilitate or enhance expression, such as a promoter, enhancer(s), destabilizing domain(s), response element(s), reporter element(s), insulator element(s), polyadenylation signal(s) and/or other functional elements. Embodiments of the invention may utilize any known suitable promoter, enhancer(s), destabilizing domain(s), response element(s), reporter element(s), insulator element(s), polyadenylation signal(s), and/or other functional elements.

As used herein, the terms “subject” and “patient” refer to an animal (e.g., a mammal, such as a human). A subject to be treated according to the methods described herein may be one who has been diagnosed with Parkinson's disease or GBA-associated Parkinson's disease, or one at risk of developing these conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the terms “transduction” and “transduce” refer to a method of introducing a vector construct or a part thereof into a cell. Wherein the vector construct is contained in a viral vector such as for example a lentiviral vector, transduction refers to viral infection of the cell, and subsequent transfer and integration of the vector construct or part thereof into the cell genome.

As used herein, “treatment” and “treating” in reference to a disease or condition, refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “vector” includes a nucleic acid vector, e.g., a DNA vector, such as a plasmid, a RNA vector, virus, or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/011026; incorporated herein by reference as it pertains to vectors suitable for the expression of a gene of interest. Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of GBA and M2-promoting agents as described herein include plasmids that contain regulatory sequences, such as promoter and enhancer regions, which direct gene transcription. Other useful vectors for expression of GBA and M2-promoting agents contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin, or zeocin.

### DETAILED DESCRIPTION

Described herein are compositions and methods for the treatment of Parkinson's disease (PD) in a subject (such as a mammalian subject, for example, a human). Using the compositions and methods described herein, one can treat Parkinson's disease (e.g., GBA-associated Parkinson's disease) in a subject (e.g., a human subject) by administering pluripotent cells, such as CD34+ cells, that express a transgene encoding glucocerebrosidase (GBA). For example, described herein are compositions containing pluripotent cells that have been modified ex-vivo to express GBA. The sections that follow describe the compositions and methods useful for the treatment of Parkinson's disease in further detail.

### Parkinson's Disease

PD is a progressive disorder that affects movement, and it is recognized as the second most common neurodegenerative disease after Alzheimer's disease. Common symptoms of PD include resting tremor, rigidity, and bradykinesia, and non-motor symptoms, such as depression, constipation, pain, sleep disorders, genitourinary problems, cognitive decline, and olfactory dysfunction, are also increasingly being associated with PD. A key feature of PD is the death of dopaminergic neurons in the substantia nigra pars compacta, and, for that reason, most current treatments for PD focus on increasing dopamine. Another well-known neuropathological hallmark of PD is the presence of Lewy bodies containing α-synuclein in brain regions affected by PD, which are thought to contribute to the disease.

PD is thought to result from a combination of genetic and environmental risk factors. There is no single gene responsible for all Parkinson's disease cases, and the vast majority of PD cases seem to be sporadic and not directly inherited. Mutations in the genes encoding parkin, PTEN-induced putative kinase 1 (PINK1), leucine-rich repeat kinase 2 (LRRK2), and Parkinsonism-associated deglycase (DJ-1) have been found to be associated with PD, but they represent only a small subset of the total number of PD cases. Occupational exposure to some pesticides and herbicides has also been proposed as a risk factor for PD. The synthetic neurotoxin MPTP can cause Parkinsonism, but its use is extremely rare.

### GBA-Associated Parkinson's Disease

Recent studies have shown a link between mutations in the GBA gene and increased risk of PD, with more severe mutations imparting higher levels of risk. Glucocerebrosidase is a lysosomal enzyme responsible for the metabolism of glucocerebroside (also known as glucosylceramide) to glucose and ceramide. It plays an important role in sphingolipid degradation, especially in the macrophage/monocyte cell lineage. Reduced GBA activity has been reported in the substantia nigra, cerebellum, and caudate of PD patients, although GBA activity has also been shown to decrease with age (see Alcalay et al., Brain 138:2648 (2015), incorporated herein by reference as it pertains to GBA activity in PD). Severely pathogenic mutations include c.84GGIns, IVS2+1 G>A, p.V394L, p.D409H, p.L444P and RecTL, which are linked to a 9.92 to 21.29 odds-ratio of developing PD. Mild GBA mutations p.N370S and p.R496H are linked to an odds-ratio of 2.84-4.94 of developing PD. The mutation p.E326K has also been identified as a PD risk factor. GBA mutations are discussed in Barkhuizen et al., Neurochemistry International 93:6 (2016) and Sidransky and Lopez, Lancet Neurol. 11:986 (2012), the disclosures of which are incorporated herein by reference as they pertain to human GBA mutations. These mutations may also elicit a gain of toxic function by activating endoplasmic reticulum (ER) stress as the mutant protein is trapped in the ER. Markers of ER stress are elevated in PD brains with GBA mutations, and dysregulation of ER calcium stores have been reported in cell models containing GBA mutations associated with PD. Additionally, these mutants could increase the total burden of to-be-degraded misfolded polypeptides in neural cells resulting in altered cellular function due to a diversion of cellular resources. GBA mutations resulting in a gain of toxic function and/or altered cellular function due to a diversion of cellular resources are discussed in Gregg et al., Ann. Neurol. 72:455-463 (2012), Schōndorf et al., Nat. Commun. 5:4028 (2014), Kilpatrick et al., Cell Calcuim. 59:12-20 (2016), and Cullen et al., Ann. Neurol. 69:940-953 (2011), the disclosure of which are incorporated herein by reference as they pertain to human GBA mutations. Studies in rodent models of PD have also suggested a link between GBA activity and α-synuclein accumulation, as described in Rocha et al., Antioxidants & Redox Signaling 23: 550 (2015) and Rocha et al., Neurobiology of Disease 82:495 (2015), the disclosures of which are disclosed herein by reference as they relate to the relationship between GBA and α-synuclein.

Treatments for Parkinson's disease have long focused on the replenishing of dopamine. Unlike these treatments, which have only treated a symptom of the disease, the compositions and methods described herein provide the benefit of treating a different biochemical phenomenon that can underlie the development of Parkinson's disease. As such, the compositions and methods described herein target the physiological cause of the disease, representing a potential curative therapy. The compositions and methods described herein can be used to treat PD by administering pluripotent cells (e.g., CD34+ cells) that express GBA. These compositions and methods can be used to treat Parkinson's disease with any etiology, e.g., genetic mutation, environmental toxin, or sporadic. These compositions and methods can also be used to treat patients with GBA-associated PD, e.g., PD associated with a mutation in the GBA gene. The compositions and methods described herein can be used to treat patients with normal GBA activity, reduced GBA activity, and patients whose GBA mutational status and/or GBA activity level is unknown. The compositions and methods described herein may also be administered as a preventative treatment to patients at risk of developing Parkinson's disease, e.g., patients with a GBA mutation, patients with reduced GBA activity, or patients with a mutation in one or more of the genes encoding parkin, PINK1, LRRK2, or DJ-1. The cells administered to patients suffering from PD can express one or more M2-promoting agents in addition to GBA.

GBA-encoding constructs that may be used in conjunction with the compositions and methods described herein include transgenes comprising polynucleotides that encode wild-type GBA (the amino acid sequence of which is shown as SEQ ID NO. 1, below) or a variant thereof, such as a polynucleotide that encodes a protein having at least 85% sequence identity (e.g., 85%, 90%, 95%, 96%, 97%, 98%, 99%, or more, sequence identity) to the amino acid sequence of SEQ ID NO. 1. In some embodiments, the GBA-encoding constructs include polynucleotides that encode the catalytic domain of GBA, such as a domain containing residues 76-381 and 416-430 of SEQ ID NO. 1. In some embodiments, the GBA-encoding constructs include polynucleotides having at least 85% sequence identity (e.g., 85%, 90%, 95%, 96%, 97%, 98%, 99%, or more, sequence identity) to the nucleic acid sequence of SEQ ID NO. 6. In some embodiments, the GBA-encoding constructs may be codon-optimized polynucleotides having at least 85% sequence identity (e.g., 85%, 90%, 95%, 96%, 97%, 98%, 99%, or more, sequence identity) to the nucleic acid sequence of SEQ ID NO. 3, so to confer resistance against degradation by nucleases and inhibitory RNAs directed to endogenous GBA. In some embodiments, the transgene encoding GBA encodes a GBA fusion protein. In some embodiments, the GBA fusion protein has an amino acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the amino acid sequence of SEQ ID NO. 2. In some embodiments, the GBA fusion protein has an amino acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the amino acid sequence of SEQ ID NO. 3. In some embodiments, the GBA fusion protein has an amino acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the amino acid sequence of SEQ ID NO. 4. In some embodiments, the transgene encoding the GBA fusion protein has a nucleic acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the nucleic acid sequence of SEQ ID NO. 8. In some embodiments, the transgene encoding the GBA fusion protein has a nucleic acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the nucleic acid sequence of SEQ ID NO. 9. In some embodiments, the transgene encoding the GBA fusion protein has a nucleic acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the nucleic acid sequence of SEQ ID NO. 10.

Wild-type human GBA (GenBank accession number: AAC63056.1) has the amino acid sequence of:

(SEQ ID NO. 1)
MEFSSPSREECPKPLSRVSIMAGSLTGLLLLQAVSWASGARPCIPKSFGY
SSVVCVCNATYCDSFDPPTFPALGTFSRYESTRSGRRMELSMGPIQANHT
GTGLLLTLQPEQKFQKVKGFGGAMTDAAALNILALSPPAQNLLLKSYFSE
EGIGYNIIRVPMASCDFSIRTYTYADTPDDFQLHNFSLPEEDTKLKIPLI
HRALQLAQRPVSLLASPWTSPTWLKTNGAVNGKGSLKGQPGDIYHQTWAR
YFVKFLDAYAEHKLQFWAVTAENEPSAGLLSGYPFQCLGFTPEHQRDFIA
RDLGPTLANSTHHNVRLLMLDDQRLLLPHWAKVVLTDPEAAKYVHGIAVH
WYLDFLAPAKATLGETHRLFPNTMLFASEACVGSKFWEQSVRLGSWDRGM
QYSHSIITNLLYHVVGWTDWNLALNPEGGPNWVRNFVDSPIIVDITKDTF
YKQPMFYHLGHFSKFIPEGSQRVGLVASQKNDLDAVALMHPDGSAVVVVL
NRSSKDVPLTIKDPAVGFLETISPGYSIHTYLWRRQ

The GBA fusion protein has the amino acid sequence of:

(SEQ ID NO. 2)
MEFSSPSREECPKPLSRVSIMAGSLTGLLLLQAVSWASGARPCIPKSFGY
SSVVCVCNATYCDSFDPPTFPALGTFSRYESTRSGRRMELSMGPIQANHT
GTGLLLTLQPEQKFQKVKGFGGAMTDAAALNILALSPPAQNLLLKSYFSE
EGIGYNIIRVPMASCDFSIRTYTYADTPDDFQLHNFSLPEEDTKLKIPLI
HRALQLAQRPVSLLASPWTSPTWLKTNGAVNGKGSLKGQPGDIYHQTWAR
YFVKFLDAYAEHKLQFWAVTAENEPSAGLLSGYPFQCLGFTPEHQRDFIA
RDLGPTLANSTHHNVRLLMLDDQRLLLPHWAKVVLTDPEAAKYVHGIAVH
WYLDFLAPAKATLGETHRLFPNTMLFASEACVGSKFWEQSVRLGSWDRGM
QYSHSIITNLLYHVVGWTDWNLALNPEGGPNWVRNFVDSPIIVDITKDTF
YKQPMFYHLGHFSKFIPEGSQRVGLVASQKNDLDAVALMHPDGSAVVVVL
NRSSKDVPLTIKDPAVGFLETISPGYSIHTYLWRRQGGGGAGGGGAGGGG
AGGGGAGGGPSLCGGELVDTLQFVCGDRGFYFSRPASRVSARSRGIVEEC
CFRSCDLALLETYCATPAKSE

Alternatively, the GBA fusion protein has the amino acid sequence of:

(SEQ ID NO. 3)
MEFSSPSREECPKPLSRVSIMAGSLTGLLLLQAVSWASGARPCIPKSFGY
SSVVCVCNATYCDSFDPPTFPALGTFSRYESTRSGRRMELSMGPIQANHT
GTGLLLTLQPEQKFQKVKGFGGAMTDAAALNILALSPPAQNLLLKSYFSE
EGIGYNIIRVPMASCDFSIRTYTYADTPDDFQLHNFSLPEEDTKLKIPLI
HRALQLAQRPVSLLASPWTSPTWLKTNGAVNGKGSLKGQPGDIYHQTWAR
YFVKFLDAYAEHKLQFWAVTAENEPSAGLLSGYPFQCLGFTPEHQRDFIA
RDLGPTLANSTHHNVRLLMLDDQRLLLPHWAKVVLTDPEAAKYVHGIAVH
WYLDFLAPAKATLGETHRLFPNTMLFASEACVGSKFWEQSVRLGSWDRGM
QYSHSIITNLLYHVVGWTDWNLALNPEGGPNWVRNFVDSPIIVDITKDTF
YKQPMFYHLGHFSKFIPEGSQRVGLVASQKNDLDAVALMHPDGSAVVVVL
NRSSKDVPLTIKDPAVGFLETISPGYSIHTYLWRRQGAPGGGSPAPAPTP
APAPTPAPAGGGPSGAPLCGGELVDTLQFVCGDRGFYFSRPASRVSARSR
GIVEECCFRSCDLALLETYCATPAKSE

Alternatively, the GBA fusion protein has the amino acid sequence of:

(SEQ ID NO. 4)
MEFSSPSREECPKPLSRVSIMAGSLTGLLLLQAVSWASGARPCIPKSFGY
SSVVCVCNATYCDSFDPPTFPALGTFSRYESTRSGRRMELSMGPIQANHT
GTGLLLTLQPEQKFQKVKGFGGAMTDAAALNILALSPPAQNLLLKSYFSE
EGIGYNIIRVPMASCDFSIRTYTYADTPDDFQLHNFSLPEEDTKLKIPLI
HRALQLAQRPVSLLASPWTSPTWLKTNGAVNGKGSLKGQPGDIYHQTWAR
YFVKFLDAYAEHKLQFWAVTAENEPSAGLLSGYPFQCLGFTPEHQRDFIA
RDLGPTLANSTHHNVRLLMLDDQRLLLPHWAKVVLTDPEAAKYVHGIAVH
WYLDFLAPAKATLGETHRLFPNTMLFASEACVGSKFWEQSVRLGSWDRGM
QYSHSIITNLLYHVVGWTDWNLALNPEGGPNWVRNFVDSPIIVDITKDTF
YKQPMFYHLGHFSKFIPEGSQRVGLVASQKNDLDAVALMHPDGSAVVVVL
NRSSKDVPLTIKDPAVGFLETISPGYSIHTYLWRRQGAPGGSPAGSPTST
EEGTSESATPESGPGTSTEPSEGSAPGSPAGSPTSTGPSGAPLCGGELVD
TLQFVCGDRGFYFSRPASRVSARSRGIVEECCFRSCDLALLETYCATPAK
SE

GBA protein having a modified signal peptide sequence has the amino acid sequence of:

(SEQ ID NO. 5)
MGIPMGKSMLVLLTFLAFASCCIAARPCIPKSFGYSSVVCVCNATYCDSF
DPPTFPALGTFSRYESTRSGRRMELSMGPIQANHTGTGLLLTLQPEQKFQ
KVKGFGGAMTDAAALNILALSPPAQNLLLKSYFSEEGIGYNIIRVPMASC
DFSIRTYTYADTPDDFQLHNFSLPEEDTKLKIPLIHRALQLAQRPVSLLA
SPWTSPTWLKTNGAVNGKGSLKGQPGDIYHQTWARYFVKFLDAYAEHKLQ
FWAVTAENEPSAGLLSGYPFQCLGFTPEHQRDFIARDLGPTLANSTHHNV
RLLMLDDQRLLLPHWAKVVLTDPEAAKYVHGIAVHWYLDFLAPAKATLGE
THRLFPNTMLFASEACVGSKFWEQSVRLGSWDRGMQYSHSIITNLLYHVV
GWTDWNLALNPEGGPNWVRNFVDSPIIVDITKDTFYKQPMFYHLGHFSKF
IPEGSQRVGLVASQKNDLDAVALMHPDGSAVVVVLNRSSKDVPLTIKDPA
VGFLETISPGYSIHTYLWRRQ

Wild-type human GBA (GenBank accession number: M19285.1) has the nucleic acid sequence of:

(SEQ ID NO. 6)
GCTAACCTAGTGCCTATAGCTAAGGCAGGTACCTGCATCCTTGTTTTTGT
TTAGTGGATCCTCTATCCTTCAGAGACTCTGGAACCCCTGTGGTCTTCTC
TTCATCTAATGACCCTGAGGGGATGGAGTTTTCAAGTCCTTCCAGAGAGG
AATGTCCCAAGCCTTTGAGTAGGGTAAGCATCATGGCTGGCAGCCTCACA
GGTTTGCTTCTACTTCAGGCAGTGTCGTGGGCATCAGGTGCCCGCCCCTG
CATCCCTAAAAGCTTCGGCTACAGCTCGGTGGTGTGTGTCTGCAATGCCA
CATACTGTGACTCCTTTGACCCCCCGACCTTTCCTGCCCTTGGTACCTTC
AGCCGCTATGAGAGTACACGCAGTGGGCGACGGATGGAGCTGAGTATGGG
GCCCATCCAGGCTAATCACACGGGCACAGGCCTGCTACTGACCCTGCAGC
CAGAACAGAAGTTCCAGAAAGTGAAGGGATTTGGAGGGGCCATGACAGAT
GCTGCTGCTCTCAACATCCTTGCCCTGTCACCCCCTGCCCAAAATTTGCT
ACTTAAATCGTACTTCTCTGAAGAAGGAATCGGATATAACATCATCCGGG
TACCCATGGCCAGCTGTGACTTCTCCATCCGCACCTACACCTATGCAGAC
ACCCCTGATGATTTCCAGTTGCACAACTTCAGCCTCCCAGAGGAAGATAC
CAAGCTCAAGATACCCCTGATTCACCGAGCCCTGCAGTTGGCCCAGCGTC
CCGTTTCACTCCTTGCCAGCCCCTGGACATCACCCACTTGGCTCAAGACC
AATGGAGCGGTGAATGGGAAGGGGTCACTCAAGGGACAGCCCGGAGACAT
CTACCACCAGACCTGGGCCAGATACTTTGTGAAGTTCCTGGATGCCTATG
CTGAGCACAAGTTACAGTTCTGGGCAGTGACAGCTGAAAATGAGCCTTCT
GCTGGGCTGTTGAGTGGATACCCCTTCCAGTGCCTGGGCTTCACCCCTGA
ACATCAGCGAGACTTCATTGCCCGTGACCTAGGTCCTACCCTCGCCAACA
GTACTCACCACAATGTCCGCCTACTCATGCTGGATGACCAACGCTTGCTG
CTGCCCCACTGGGCAAAGGTGGTACTGACAGACCCAGAAGCAGCTAAATA
TGTTCATGGCATTGCTGTACATTGGTACCTGGACTTTCTGGCTCCAGCCA
AAGCCACCCTAGGGGAGACACACCGCCTGTTCCCCAACACCATGCTCTTT
GCCTCAGAGGCCTGTGTGGGCTCCAAGTTCTGGGAGCAGAGTGTGCGGCT
AGGCTCCTGGGATCGAGGGATGCAGTACAGCCACAGCATCATCACGAACC
TCCTGTACCATGTGGTCGGCTGGACCGACTGGAACCTTGCCCTGAACCCC
GAAGGAGGACCCAATTGGGTGCGTAACTTTGTCGACAGTCCCATCATTGT
AGACATCACCAAGGACACGTTTTACAAACAGCCCATGTTCTACCACCTTG
GCCACTTCAGCAAGTTCATTCCTGAGGGCTCCCAGAGAGTGGGGCTGGTT
GCCAGTCAGAAGAACGACCTGGACGCAGTGGCACTGATGCATCCCGATGG
CTCTGCTGTTGTGGTCGTGCTAAACCGCTCCTCTAAGGATGTGCCTCTTA
CCATCAAGGATCCTGCTGTGGGCTTCCTGGAGACAATCTCACCTGGCTAC
TCCATTCACACCTACCTGTGGCATCGCCAGTGATGGAGCAGATACTCAAG
GAGGCACTGGGCTCAGCCTGGGCATTAAAGGGACAGAGTCAGCTCACACG
CTGTCTGTGACTAAAGAGGGCACAGCAGGGCCAGTGTGAGCTTACAGCGA
CGTAAGCCCAGGGGCAATGGTTTGGGTGACTCACTTTCCCCTCTAGGTGG
TGCCCAGGGCTGGAGGCCCCTAGAAAAAGATCAGTAAGCCCCAGTGTCCC
CCCAGCCCCCATGCTTATGTGAACATGCGCTGTGTGCTGCTTGCTTTGGA
AACTNGCCTGGGTCCAGGCCTAGGGTGAGCTCACTGTCCGTACAAACACA
AGATCAGGGCTGAGGGTAAGGAAAAGAAGAGACTAGGAAAGCTGGGCCCA
AAACTGGAGACTGTTTGTCTTTCCTAGAGATGCAGAACTGGGCCCGTGGA
GCAGCAGTGTCAGCATCAGGGCGGAAGCCTTAAAGCAGCAGCGGGTGTGC
CCAGGCACCCAGATGATTCCTATGGCACCAGCCAGGAAAAATGGCAGCTC
TTAAAGGAGAAAATGTTTGAGCCCA

The codon-optimized GBA construct has the nucleic acid sequence of:

(SEQ ID NO. 7)
ATGGAATTCAGCAGCCCCAGCCGGGAGGAATGCCCCAAGCCCCTGAGCAG
AGTGTCCATCATGGCCGGATCTCTGACCGGACTGCTGCTGCTGCAAGCCG
TGTCTTGGGCCAGCGGCGCCAGACCTTGCATCCCCAAGAGCTTCGGCTAC
AGCAGCGTCGTGTGCGTGTGCAACGCCACCTACTGCGACAGCTTCGACCC
CCCTACCTTTCCCGCCCTGGGCACCTTCAGCAGATACGAGAGCACCCGCA
GCGGCAGACGGATGGAACTGAGCATGGGCCCCATCCAGGCCAATCACACC
GGAACCGGCCTGCTGCTGACCCTGCAGCCCGAGCAGAAATTCCAGAAAGT
GAAGGGCTTCGGCGGAGCCATGACAGACGCCGCTGCCCTGAACATCCTGG
CCCTGAGCCCCCCTGCTCAGAATCTGCTGCTCAAGAGCTACTTCAGCGAG
GAAGGCATCGGCTACAACATCATCCGGGTGCCCATGGCTAGCTGCGACTT
CAGCATCCGGACCTACACCTACGCCGACACCCCCGACGACTTCCAGCTGC
ACAACTTCAGCCTGCCCGAAGAGGACACCAAGCTGAAGATCCCCCTGATC
CACAGAGCCCTGCAGCTGGCCCAGAGGCCTGTGTCTCTGCTGGCTAGCCC
TTGGACCAGCCCCACCTGGCTGAAAACCAACGGAGCCGTGAACGGCAAGG
GCAGCCTGAAGGGCCAGCCCGGCGACATCTACCACCAGACCTGGGCCCGG
TACTTCGTGAAGTTCCTGGACGCCTATGCCGAGCACAAGCTGCAGTTCTG
GGCCGTGACCGCCGAGAATGAGCCTAGCGCTGGACTGCTGTCCGGCTACC
CCTTCCAATGCCTGGGCTTCACACCCGAGCACCAGCGGGACTTTATCGCC
AGAGATCTGGGCCCCACACTGGCCAACAGCACCCACCACAACGTGCGGCT
GCTGATGCTGGACGACCAGAGACTGCTGCTCCCCCACTGGGCCAAGGTGG
TGCTGACAGACCCCGAGGCCGCCAAATACGTCCACGGGATTGCCGTGCAC
TGGTATCTGGACTTTCTGGCCCCTGCCAAGGCCACACTGGGCGAGACACA
CCGGCTGTTCCCCAACACCATGCTGTTCGCCAGCGAGGCCTGCGTGGGCA
GCAAGTTCTGGGAGCAGAGCGTGCGGCTGGGCAGCTGGGACAGAGGCATG
CAGTACAGCCACAGCATCATCACCAACCTGCTGTACCACGTCGTGGGCTG
GACCGACTGGAATCTGGCCCTGAACCCTGAGGGAGGACCCAACTGGGTCC
GCAACTTCGTGGACAGCCCCATCATCGTGGACATCACCAAGGACACCTTC
TACAAGCAGCCCATGTTCTACCACCTGGGCCACTTCAGCAAGTTCATCCC
CGAGGGCAGCCAGAGAGTGGGACTGGTCGCCAGCCAGAAGAACGATCTGG
ACGCCGTGGCCCTGATGCACCCTGATGGCAGCGCTGTGGTGGTGGTCCTG
AATCGGTCCAGCAAGGACGTGCCCCTGACCATCAAGGACCCCGCCGTGGG
CTTCCTGGAAACCATCAGCCCCGGCTACTCCATCCACACCTACCTGTGGC
GGAGACAATGA

The codon-optimized GBA fusion protein construct has the nucleic acid sequence of:

(SEQ ID NO. 8)
ATGGAATTCAGCAGCCCCAGCCGGGAGGAATGCCCCAAGCCCCTGAGCAG
AGTGTCCATCATGGCCGGATCTCTGACCGGACTGCTGCTGCTGCAAGCCG
TGTCTTGGGCCAGCGGCGCCAGACCTTGCATCCCCAAGAGCTTCGGCTAC
AGCAGCGTCGTGTGCGTGTGCAACGCCACCTACTGCGACAGCTTCGACCC
CCCTACCTTTCCCGCCCTGGGCACCTTCAGCAGATACGAGAGCACCCGCA
GCGGCAGACGGATGGAACTGAGCATGGGCCCCATCCAGGCCAATCACACC
GGAACCGGCCTGCTGCTGACCCTGCAGCCCGAGCAGAAATTCCAGAAAGT
GAAGGGCTTCGGCGGAGCCATGACAGACGCCGCTGCCCTGAACATCCTGG
CCCTGAGCCCCCCTGCTCAGAATCTGCTGCTCAAGAGCTACTTCAGCGAG
GAAGGCATCGGCTACAACATCATCCGGGTGCCCATGGCTAGCTGCGACTT
CAGCATCCGGACCTACACCTACGCCGACACCCCCGACGACTTCCAGCTGC
ACAACTTCAGCCTGCCCGAAGAGGACACCAAGCTGAAGATCCCCCTGATC
CACAGAGCCCTGCAGCTGGCCCAGAGGCCTGTGTCTCTGCTGGCTAGCCC
TTGGACCAGCCCCACCTGGCTGAAAACCAACGGAGCCGTGAACGGCAAGG
GCAGCCTGAAGGGCCAGCCCGGCGACATCTACCACCAGACCTGGGCCCGG
TACTTCGTGAAGTTCCTGGACGCCTATGCCGAGCACAAGCTGCAGTTCTG
GGCCGTGACCGCCGAGAATGAGCCTAGCGCTGGACTGCTGTCCGGCTACC
CCTTCCAATGCCTGGGCTTCACACCCGAGCACCAGCGGGACTTTATCGCC
AGAGATCTGGGCCCCACACTGGCCAACAGCACCCACCACAACGTGCGGCT
GCTGATGCTGGACGACCAGAGACTGCTGCTCCCCCACTGGGCCAAGGTGG
TGCTGACAGACCCCGAGGCCGCCAAATACGTCCACGGGATTGCCGTGCAC
TGGTATCTGGACTTTCTGGCCCCTGCCAAGGCCACACTGGGCGAGACACA
CCGGCTGTTCCCCAACACCATGCTGTTCGCCAGCGAGGCCTGCGTGGGCA
GCAAGTTCTGGGAGCAGAGCGTGCGGCTGGGCAGCTGGGACAGAGGCATG
CAGTACAGCCACAGCATCATCACCAACCTGCTGTACCACGTCGTGGGCTG
GACCGACTGGAATCTGGCCCTGAACCCTGAGGGAGGACCCAACTGGGTCC
GCAACTTCGTGGACAGCCCCATCATCGTGGACATCACCAAGGACACCTTC
TACAAGCAGCCCATGTTCTACCACCTGGGCCACTTCAGCAAGTTCATCCC
CGAGGGCAGCCAGAGAGTGGGACTGGTCGCCAGCCAGAAGAACGATCTGG
ACGCCGTGGCCCTGATGCACCCTGATGGCAGCGCTGTGGTGGTGGTCCTG
AATCGGTCCAGCAAGGACGTGCCCCTGACCATCAAGGACCCCGCCGTGGG
CTTCCTGGAAACCATCAGCCCCGGCTACTCCATCCACACCTACCTGTGGC
GGAGACAAGGCGGAGGCGGAGCTGGTGGCGGCGGAGCAGGCGGTGGTGGT
GCAGGCGGCGGAGGTGCTGGCGGAGGACCATCTCTTTGTGGCGGAGAACT
GGTGGACACCCTGCAGTTCGTGTGTGGCGACAGAGGCTTCTACTTTAGCA
GACCCGCCAGCAGAGTGTCCGCCAGATCTAGAGGAATCGTGGAAGAGTGC
TGCTTCAGAAGCTGCGACCTGGCACTGCTGGAAACCTACTGTGCCACACC
AGCCAAGAGCGAGTGATGA

Alternatively, the codon-optimized human GBA fusion protein has the nucleic acid sequence of:

(SEQ ID NO. 9)
ATGGAATTCAGCAGCCCCAGCCGGGAGGAATGCCCCAAGCCCCTGAGCAG
AGTGTCCATCATGGCCGGATCTCTGACCGGACTGCTGCTGCTGCAAGCCG
TGTCTTGGGCCAGCGGCGCCAGACCTTGCATCCCCAAGAGCTTCGGCTAC
AGCAGCGTCGTGTGCGTGTGCAACGCCACCTACTGCGACAGCTTCGACCC
CCCTACCTTTCCCGCCCTGGGCACCTTCAGCAGATACGAGAGCACCCGCA
GCGGCAGACGGATGGAACTGAGCATGGGCCCCATCCAGGCCAATCACACC
GGAACCGGCCTGCTGCTGACCCTGCAGCCCGAGCAGAAATTCCAGAAAGT
GAAGGGCTTCGGCGGAGCCATGACAGACGCCGCTGCCCTGAACATCCTGG
CCCTGAGCCCCCCTGCTCAGAATCTGCTGCTCAAGAGCTACTTCAGCGAG
GAAGGCATCGGCTACAACATCATCCGGGTGCCCATGGCTAGCTGCGACTT
CAGCATCCGGACCTACACCTACGCCGACACCCCCGACGACTTCCAGCTGC
ACAACTTCAGCCTGCCCGAAGAGGACACCAAGCTGAAGATCCCCCTGATC
CACAGAGCCCTGCAGCTGGCCCAGAGGCCTGTGTCTCTGCTGGCTAGCCC
TTGGACCAGCCCCACCTGGCTGAAAACCAACGGAGCCGTGAACGGCAAGG
GCAGCCTGAAGGGCCAGCCCGGCGACATCTACCACCAGACCTGGGCCCGG
TACTTCGTGAAGTTCCTGGACGCCTATGCCGAGCACAAGCTGCAGTTCTG
GGCCGTGACCGCCGAGAATGAGCCTAGCGCTGGACTGCTGTCCGGCTACC
CCTTCCAATGCCTGGGCTTCACACCCGAGCACCAGCGGGACTTTATCGCC
AGAGATCTGGGCCCCACACTGGCCAACAGCACCCACCACAACGTGCGGCT
GCTGATGCTGGACGACCAGAGACTGCTGCTCCCCCACTGGGCCAAGGTGG
TGCTGACAGACCCCGAGGCCGCCAAATACGTCCACGGGATTGCCGTGCAC
TGGTATCTGGACTTTCTGGCCCCTGCCAAGGCCACACTGGGCGAGACACA
CCGGCTGTTCCCCAACACCATGCTGTTCGCCAGCGAGGCCTGCGTGGGCA
GCAAGTTCTGGGAGCAGAGCGTGCGGCTGGGCAGCTGGGACAGAGGCATG
CAGTACAGCCACAGCATCATCACCAACCTGCTGTACCACGTCGTGGGCTG
GACCGACTGGAATCTGGCCCTGAACCCTGAGGGAGGACCCAACTGGGTCC
GCAACTTCGTGGACAGCCCCATCATCGTGGACATCACCAAGGACACCTTC
TACAAGCAGCCCATGTTCTACCACCTGGGCCACTTCAGCAAGTTCATCCC
CGAGGGCAGCCAGAGAGTGGGACTGGTCGCCAGCCAGAAGAACGATCTGG
ACGCCGTGGCCCTGATGCACCCTGATGGCAGCGCTGTGGTGGTGGTCCTG
AATCGGTCCAGCAAGGACGTGCCCCTGACCATCAAGGACCCCGCCGTGGG
CTTCCTGGAAACCATCAGCCCCGGCTACTCCATCCACACCTACCTGTGGC
GGAGACAAGGAGCACCAGGCGGAGGATCTCCAGCTCCTGCTCCTACACCA
GCTCCAGCACCGACGCCTGCTCCAGCTGGCGGAGGACCTTCTGGTGCACC
TCTTTGTGGCGGAGAGCTGGTGGATACCCTGCAGTTCGTGTGTGGCGACC
GGGGCTTCTACTTTAGCAGACCTGCCAGCAGAGTGTCCGCCAGATCTAGA
GGCATCGTGGAAGAGTGCTGCTTCAGAAGCTGCGACCTGGCACTGCTGGA
AACCTACTGTGCCACACCAGCCAAGAGCGAGTGATGA

Alternatively, the codon-optimized human GBA fusion protein has the nucleic acid sequence of:

(SEQ ID NO. 10)
ATGGAATTCAGCAGCCCCAGCCGGGAGGAATGCCCCAAGCCCCTGAGCAG
AGTGTCCATCATGGCCGGATCTCTGACCGGACTGCTGCTGCTGCAAGCCG
TGTCTTGGGCCAGCGGCGCCAGACCTTGCATCCCCAAGAGCTTCGGCTAC
AGCAGCGTCGTGTGCGTGTGCAACGCCACCTACTGCGACAGCTTCGACCC
CCCTACCTTTCCCGCCCTGGGCACCTTCAGCAGATACGAGAGCACCCGCA
GCGGCAGACGGATGGAACTGAGCATGGGCCCCATCCAGGCCAATCACACC
GGAACCGGCCTGCTGCTGACCCTGCAGCCCGAGCAGAAATTCCAGAAAGT
GAAGGGCTTCGGCGGAGCCATGACAGACGCCGCTGCCCTGAACATCCTGG
CCCTGAGCCCCCCTGCTCAGAATCTGCTGCTCAAGAGCTACTTCAGCGAG
GAAGGCATCGGCTACAACATCATCCGGGTGCCCATGGCTAGCTGCGACTT
CAGCATCCGGACCTACACCTACGCCGACACCCCCGACGACTTCCAGCTGC
ACAACTTCAGCCTGCCCGAAGAGGACACCAAGCTGAAGATCCCCCTGATC
CACAGAGCCCTGCAGCTGGCCCAGAGGCCTGTGTCTCTGCTGGCTAGCCC
TTGGACCAGCCCCACCTGGCTGAAAACCAACGGAGCCGTGAACGGCAAGG
GCAGCCTGAAGGGCCAGCCCGGCGACATCTACCACCAGACCTGGGCCCGG
TACTTCGTGAAGTTCCTGGACGCCTATGCCGAGCACAAGCTGCAGTTCTG
GGCCGTGACCGCCGAGAATGAGCCTAGCGCTGGACTGCTGTCCGGCTACC
CCTTCCAATGCCTGGGCTTCACACCCGAGCACCAGCGGGACTTTATCGCC
AGAGATCTGGGCCCCACACTGGCCAACAGCACCCACCACAACGTGCGGCT
GCTGATGCTGGACGACCAGAGACTGCTGCTCCCCCACTGGGCCAAGGTGG
TGCTGACAGACCCCGAGGCCGCCAAATACGTCCACGGGATTGCCGTGCAC
TGGTATCTGGACTTTCTGGCCCCTGCCAAGGCCACACTGGGCGAGACACA
CCGGCTGTTCCCCAACACCATGCTGTTCGCCAGCGAGGCCTGCGTGGGCA
GCAAGTTCTGGGAGCAGAGCGTGCGGCTGGGCAGCTGGGACAGAGGCATG
CAGTACAGCCACAGCATCATCACCAACCTGCTGTACCACGTCGTGGGCTG
GACCGACTGGAATCTGGCCCTGAACCCTGAGGGAGGACCCAACTGGGTCC
GCAACTTCGTGGACAGCCCCATCATCGTGGACATCACCAAGGACACCTTC
TACAAGCAGCCCATGTTCTACCACCTGGGCCACTTCAGCAAGTTCATCCC
CGAGGGCAGCCAGAGAGTGGGACTGGTCGCCAGCCAGAAGAACGATCTGG
ACGCCGTGGCCCTGATGCACCCTGATGGCAGCGCTGTGGTGGTGGTCCTG
AATCGGTCCAGCAAGGACGTGCCCCTGACCATCAAGGACCCCGCCGTGGG
CTTCCTGGAAACCATCAGCCCCGGCTACTCCATCCACACCTACCTGTGGC
GGAGACAAGGAGCACCAGGCGGATCTCCAGCAGGATCTCCAACCTCTACC
GAGGAAGGCACAAGCGAGTCTGCCACACCTGAGTCTGGACCTGGCACAAG
CACAGAGCCTAGCGAAGGATCTGCCCCAGGTTCTCCTGCCGGCTCTCCTA
CAAGTACAGGACCTTCTGGCGCTCCACTGTGTGGCGGAGAACTGGTGGAT
ACCCTGCAGTTCGTGTGCGGCGACAGAGGCTTCTACTTTAGCAGACCCGC
CAGCAGAGTGTCCGCCAGATCTAGAGGAATCGTGGAAGAGTGCTGCTTCA
GAAGCTGCGATCTGGCACTGCTGGAAACCTACTGTGCCACACCAGCCAAG
AGCGAGTGATGA

The codon-optimized human GBA having a modified signal peptide sequence has the nucleic acid sequence of:

(SEQ ID NO. 11)
ATGGGGATTCCTATGGGCAAGTCTATGCTGGTCCTGCTGACATTTCTGGC
CTTCGCTTCATGCTGTATCGCTGCCAGACCTTGCATCCCCAAGAGCTTCG
GCTACAGCAGCGTCGTGTGCGTGTGCAACGCCACCTACTGCGACAGCTTC
GACCCCCCTACCTTTCCCGCCCTGGGCACCTTCAGCAGATACGAGAGCAC
CCGCAGCGGCAGACGGATGGAACTGAGCATGGGCCCCATCCAGGCCAATC
ACACCGGAACCGGCCTGCTGCTGACCCTGCAGCCCGAGCAGAAATTCCAG
AAAGTGAAGGGCTTCGGCGGAGCCATGACAGACGCCGCTGCCCTGAACAT
CCTGGCCCTGAGCCCCCCTGCTCAGAATCTGCTGCTCAAGAGCTACTTCA
GCGAGGAAGGCATCGGCTACAACATCATCCGGGTGCCCATGGCTAGCTGC
GACTTCAGCATCCGGACCTACACCTACGCCGACACCCCCGACGACTTCCA
GCTGCACAACTTCAGCCTGCCCGAAGAGGACACCAAGCTGAAGATCCCCC
TGATCCACAGAGCCCTGCAGCTGGCCCAGAGGCCTGTGTCTCTGCTGGCT
AGCCCTTGGACCAGCCCCACCTGGCTGAAAACCAACGGAGCCGTGAACGG
CAAGGGCAGCCTGAAGGGCCAGCCCGGCGACATCTACCACCAGACCTGGG
CCCGGTACTTCGTGAAGTTCCTGGACGCCTATGCCGAGCACAAGCTGCAG
TTCTGGGCCGTGACCGCCGAGAATGAGCCTAGCGCTGGACTGCTGTCCGG
CTACCCCTTCCAATGCCTGGGCTTCACACCCGAGCACCAGCGGGACTTTA
TCGCCAGAGATCTGGGCCCCACACTGGCCAACAGCACCCACCACAACGTG
CGGCTGCTGATGCTGGACGACCAGAGACTGCTGCTCCCCCACTGGGCCAA
GGTGGTGCTGACAGACCCCGAGGCCGCCAAATACGTCCACGGGATTGCCG
TGCACTGGTATCTGGACTTTCTGGCCCCTGCCAAGGCCACACTGGGCGAG
ACACACCGGCTGTTCCCCAACACCATGCTGTTCGCCAGCGAGGCCTGCGT
GGGCAGCAAGTTCTGGGAGCAGAGCGTGCGGCTGGGCAGCTGGGACAGAG
GCATGCAGTACAGCCACAGCATCATCACCAACCTGCTGTACCACGTCGTG
GGCTGGACCGACTGGAATCTGGCCCTGAACCCTGAGGGAGGACCCAACTG
GGTCCGCAACTTCGTGGACAGCCCCATCATCGTGGACATCACCAAGGACA
CCTTCTACAAGCAGCCCATGTTCTACCACCTGGGCCACTTCAGCAAGTTC
ATCCCCGAGGGCAGCCAGAGAGTGGGACTGGTCGCCAGCCAGAAGAACGA
TCTGGACGCCGTGGCCCTGATGCACCCTGATGGCAGCGCTGTGGTGGTGG
TCCTGAATCGGTCCAGCAAGGACGTGCCCCTGACCATCAAGGACCCCGCC
GTGGGCTTCCTGGAAACCATCAGCCCCGGCTACTCCATCCACACCTACCT
GTGGCGGAGACAATGA

In some embodiments, the GBA-encoding constructs include polynucleotides that encode wild-type GBA with the natural signal peptide (a 39-amino acid GBA signal peptide as described in Sorge et al., Am. J. Hum. Genet. 41:1016-1024 (1987)). In some embodiments, the GBA-encoding constructs include polynucleotides that encode wild-type GBA without the natural signal peptide. In some embodiments, the transgene encoding secreted GBA comprises a modified signal peptide. In some embodiments, the GBA comprising a modified signal peptide has an amino acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the amino acid sequence of SEQ ID NO. 5. In some embodiments, the transgene encoding secreted GBA comprising a modified signal peptide has a nucleic acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the nucleic acid sequence of SEQ ID NO. 11. In some embodiments, the modified signal peptide has an amino acid sequence having at least 85% sequence identity (e.g., 85%, 86%, 87%, 88%, 89%, 90%, 91%, 92%, 93%, 94%, 95%, 96%, 97%, 98%, 99%, or 100% sequence identity) to the amino acid sequence of SEQ ID NO. 16, as shown below.

(SEQ ID NO. 16)
MGIPMGKSMLVLLTFLAFASCCIA

In some embodiments, the modified signal peptide is encoded by a polynucleotide having at least 85% sequence identity (e.g., 85%, 86%, 87%, 88%, 89%, 90%, 91%, 92%, 93%, 94%, 95%, 96%, 97%, 98%, 99%, or 100% sequence identity) to the nucleic acid sequence of SEQ ID NO. 20, as shown below.

(SEQ ID NO. 20)
ATGGGGATTCCTATGGGCAAGTCTATGCTGGTCCTGCTGACATTTCTGG
CCTTCGCTTCATGCTGTATCGCT

According to the methods described herein, a patient can be administered a pluripotent cell (e.g., an HSC, iPSC, CD34+ cell, ES cell, or myeloid progenitor cell) that expresses a polynucleotide encoding the amino acid sequence of SEQ ID NO. 1, or a polynucleotide encoding a polypeptide having at least 85% sequence identity (e.g., 85%, 90%, 95%, 96%, 97%, 98%, 99%, or more, sequence identity) to the amino acid sequence of SEQ ID NO. 1, or a polynucleotide encoding a polypeptide that contains one or more conservative amino acid substitutions relative to SEQ ID NO. 1 (e.g., 1, 2, 3, 4, 5, 6, 7, 8, 9, or 10 or more conservative amino acid substitutions), provided that the GBA analog encoded retains the therapeutic function of wild-type GBA. The enzymatic activity of wild-type GBA is important for the metabolism of glycolipids. GBA acts in lysosomes to metabolize the sphingolipid glucocerebroside to glucose and ceramide. Loss of GBA function leads to an accumulation of glucocerebroside in macrophages.

### Host Cells

Cells that may be used in conjunction with the compositions and methods described herein include cells that are capable of undergoing further differentiation. For example, one type of cell that can be used in conjunction with the compositions and methods described herein is a pluripotent cell (e.g., a CD34+ cell). A pluripotent cell is a cell that possesses the ability to develop into more than one differentiated cell type. Examples of pluripotent cells are embryonic stem cells (ES cells) and induced pluripotent stem cells (iPS cells or iPSCs). ES cells and iPS cells have the ability to differentiate into cells of the ectoderm, which gives rise to the skin and nervous system, endoderm, which forms the gastrointestinal and respiratory tracts, endocrine glands, liver, and pancreas, and mesoderm, which forms bone, cartilage, muscles, connective tissue, and most of the circulatory system.

Cells that may be used in conjunction with the compositions and methods described herein include hematopoietic stem cells and myeloid progenitor cells. Hematopoietic stem cells (HSCs) are immature blood cells that have the capacity to self-renew and to differentiate into mature blood cells including diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Human HSCs are CD34+. In addition, HSCs also refer to long term repopulating HSC (LT-HSC) and short-term repopulating HSC (ST-HSC). Any of these HSCs can be used in conjunction with the compositions and methods described herein.

HSCs can differentiate into myeloid progenitor cells, which are also CD34+. Myeloid progenitors can further differentiate into granulocytes (e.g., promyelocytes, neutrophils, eosinophils, and basophils), erythrocytes (e.g., reticulocytes, erythrocytes), thrombocytes (e.g., megakaryoblasts, platelet producing megakaryocytes, and platelets), monocytes (e.g., monocytes and macrophages), dendritic cells, and microglia. Common myeloid progenitors can be characterized by cell surface molecules and are known to be lin−, SCA1−, c-kit+, CD34+, and CD16/32^{mid}.

HSCs and myeloid progenitors can be obtained from blood products. A blood product is a product obtained from the body or an organ of the body containing cells of hematopoietic origin. Such sources include unfractionated bone marrow, umbilical cord, placenta, peripheral blood, or mobilized-peripheral blood. All of the aforementioned crude or unfractionated blood products can be enriched for cells having HSC or myeloid progenitor cell characteristics in a number of ways. For example, the more mature, differentiated cells can be selected against based on cell surface molecules they express. The blood product may be fractionated by positively selecting for CD34+ cells, which include a subpopulation of hematopoietic stem cells capable of self-renewal, multi-potency, and that can be re-introduced into a transplant recipient whereupon they home to the hematopoietic stem cell niche and reestablish productive and sustained hematopoiesis. Such selection is accomplished using, for example, commercially available magnetic anti-CD34 beads (Dynal, Lake Success, NY). Myeloid progenitor cells can also be isolated based on the markers they express. Unfractionated blood products can be obtained directly from a donor or retrieved from cryopreservative storage. HSCs and myeloid progenitor cells can also be obtained from by differentiation of ES cells, iPS cells or other reprogrammed mature cells types.

Cells that may be used in conjunction with the compositions and methods described herein include allogeneic cells or autologous cells. All of the aforementioned cell types are capable of differentiating into microglia. Cells may also differentiate into microglial progenitors or microglial stem cells. Differentiation may occur ex vivo or in vivo. Methods for ex vivo differentiation of human ES cells and iPS cells are known by those of skill in the art and are described in Muffat et al., Nature Medicine 22:1358-1367 (2016) and Pandya et al., Nature Neuroscience (2017) epub ahead of print, the disclosures of which are incorporated herein by reference as they pertain to methods of differentiating pluripotent cells into microglia.

### Microglia

Cells that may be used in conjunction with the compositions and methods described herein include those that are capable of differentiating into microglial cells. Microglia are myeloid-derived cells that serve as the immune cells, or resident macrophages, of the central nervous system. Microglia are highly similar to macrophages, both genetically and functionally, and share the ability to shift dynamically between pro-inflammatory and anti-inflammatory states. The pro-inflammatory state is known as classical activation, or M1, and the anti-inflammatory state is called alternative activation, or M2. Microglia can be made to shift between the two states by extracellular signals, e.g., signals from neighboring neurons or astrocytes, cell debris, toxins, infection, ischemia, and traumatic injury, among others. M1 microglia are often observed in the diseased brain, particularly in diseases involving neuroinflammation, such as PD. Classically activated M1 phenotypes have also been observed in mouse models of PD, such as the parkin null mouse and DJ-1 null mouse. It is unclear whether M1 microglia are a cause or consequence of neuroinflammation, but once microglia are classically activated, they can secrete pro-inflammatory cytokines, e.g., TNF-α, IL-1β, and IL-6, chemokines, and nitric oxide, which can lead to sustained inflammation, neuronal damage, and further activation of M1 microglia. This positive feedback loop can be harmful to brain tissue; therefore, methods of reducing M1 activation and/or increasing M2 activation may help patients with diseases featuring neuroinflammation, e.g., Parkinson's disease.

### M2 Promoting Agents

The compositions and methods described herein additionally provide cells expressing GBA and one or more M2-promoting agents. An M2-promoting agent is an agent that affects the polarization of macrophages or microglia by shifting cells toward a more anti-inflammatory state. M2-promoting agents can reduce M1 microglia numbers or the M1 microglia phenotype by reducing secretion or expression of pro-inflammatory cytokines, chemokines, and genes associated with classical activation/M1, e.g., IL-1β, tumor necrosis factor, iNOS, IL-6, or IL-12. M2-promoting agents may also reduce markers of M1 cells, e.g., MHC II, CD86, CD68, CD11B, and Fcγ receptors. M2-promoting agents may increase the number of M2 microglia or the M2 phenotype by increasing secretion and expression of anti-inflammatory cytokines and genes associated with alternative activation/M2, e.g., IL-25, IL-10, transforming growth factor beta, CD163, TREM2, Arg1, or CD206. M2-promoting agents may also alter cell morphology or shape, e.g., reducing amoeboid microglia or increasing microglial ramification. The M2 agents used in the compositions and methods described herein may make the administered cells display an M2 phenotype, may induce microglia in the brain to shift away from an M1 phenotype or toward an M2 phenotype, or may affect the phenotype of both the administered cells and the endogenous microglia. Microglia are rarely observed in a strictly M1 or M2 state and often express a subset of both M1 and M2 markers. A shift toward an M2 phenotype may appear as a reduction in one or more M1-associated cytokines or genes, an increase in one or more M2-associated cytokines and genes, or a change in both M1- and M2-associated cytokines and genes.

M2-promoting agents fall into a number of broad categories. Agents that promote an M2 phenotype or reduce an M1 phenotype include anti-inflammatory cytokines, e.g., interleukin-25 (IL-25), interleukin-4 (IL-4), interleukin-10 (IL-10), interleukin-13 (IL-13), and transforming growth factor beta (TGF-β). Expression of cytokine receptors, such as IL-4R, IL-17RB, IL-10R, TGFβR1, and TGFβR2 can also promote an M2 phenotype by enabling cells to detect anti-inflammatory cytokines. IL-25 is an anti-inflammatory cytokine shown to inhibit neuroinflammation. It is produced by several cells, such as Type 2 helper T (Th2) cells, mast cells, epithelial cells, eosinophils, basophils and alveolar macrophages, and has been shown to shift microglia to a more anti-inflammatory, M2 state, as described in Maiorino et al., Gene Therapy 20:487 (2013), the disclosure of which is incorporated herein by reference as it pertains to IL-25 as an M2-promoting agent.

Another category of M2-promoting agents includes agents that reduce expression, synthesis, and secretion of pro-inflammatory mediators, e.g., cytokines, nitrite, and NF-κB activity. Agents in this category include glucocorticoid receptors, e.g., GR, peroxisome proliferator-activated receptors (PPARs), e.g., PPARγ, PPARβ/δ, estrogen receptors, e.g., ERα, ERβ, and nuclear receptor subfamily 4 group A member 2 (NR4A2). M2-promoting agents can induce an M2 phenotype by causing epigenetic changes, and agents in this class include lysine demethylase 6B (KDM6B), MSH homeobox 3 (MSX3), family with sequence similarity 19 (chemokine (C—C motif)-like), member A3 (FAM19A3), nuclear factor NF-Kappa-B P50 subunit (NF-κB p50). microRNAs, including miR124, miR21, and miR181c, can also serve as M2-promoting agents. Another category of agents that can promote an M2 phenotype or reduce an M1 phenotype are agents known as self-associated molecular patterns (SAMPs) that are known from the immune system for helping phagocytes discriminate self from non-self material. Agents in this class include C-X3-C motif chemokine ligand 1 (CX3CL1), C-X3-C motif chemokine receptor 1 (CX3CR1), CD200 molecule (CD200), CD200 receptor 1 (CD200R), complement factor H (CFH), leukocyte surface antigen CD47 (CD47), complement decay-accelerating factor (CD55), trophoblast leukocyte common antigen (CD46), adhesion G protein-coupled receptor E5 (ADGRE5), signal regulatory protein alpha (SIRPA), and siglecs, e.g., Siglec-1, Siglec-2, Siglec-3, Siglec-4, CD33-related Siglecs. For a discussion of microglial protective regulatory signals, see Le et al., Frontiers in Molecular Neuroscience 9 (2016) online publication, the disclosure of which is incorporated herein by reference as it pertains to signals that reduce classical activation of microglia.

### Expression of GBA and M2-Promoting Agents in Mammalian Cells

Glucocerebrosidase activity is reduced in patients with Parkinson's disease, and PD brains contain classically activated M1 microglia. The compositions and methods described herein target these dysfunctions by administering cells expressing a transgene encoding GBA (e.g., non-secreted GBA or secreted GBA) and, optionally, one or more transgenes that each encode an M2-promoting agent. In order to utilize these agents for therapeutic application in the treatment of Parkinson's disease, these agents can be directed to the interior of the cell, and in particular examples, to particular organelles. A wide array of methods has been established for the delivery of such proteins to mammalian cells and for the stable expression of genes encoding such proteins in mammalian cells.

### Polynucleotides Encoding GBA and M2-Promoting Agents

One platform that can be used to achieve therapeutically effective intracellular concentrations of GBA and M2-promoting agents in mammalian cells is via the stable expression of genes encoding these agents (e.g., by integration into the nuclear or mitochondrial genome of a mammalian cell). These genes are polynucleotides that encode the primary amino acid sequence of the corresponding protein. In order to introduce such exogenous genes into a mammalian cell, these genes can be incorporated into a vector. Vectors can be introduced into a cell by a variety of methods, including transformation, transfection, direct uptake, projectile bombardment, and by encapsulation of the vector in liposomes. Examples of suitable methods of transfecting or transforming cells are calcium phosphate precipitation, electroporation, microinjection, infection, lipofection, and direct uptake. Such methods are described in more detail, for example, in Green et al., Molecular Cloning: A Laboratory Manual, Fourth Edition (Cold Spring Harbor University Press, New York (2014)); and Ausubel et al., Current Protocols in Molecular Biology (John Wiley & Sons, New York (2015)), the disclosures of each of which are incorporated herein by reference.

GBA and M2-promoting agents can also be introduced into a mammalian cell by targeting a vector containing a gene encoding such an agent to cell membrane phospholipids. For example, vectors can be targeted to the phospholipids on the extracellular surface of the cell membrane by linking the vector molecule to a VSV-G protein, a viral protein with affinity for all cell membrane phospholipids. Such a construct can be produced using methods well known to those of skill in the field.

Recognition and binding of the polynucleotide encoding GBA and/or an M2-promoting agent by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase. Examples of mammalian promoters have been described in Smith et al., Mol. Sys. Biol., 3:73, online publication, the disclosure of which is incorporated herein by reference.

Polynucleotides suitable for use with the compositions and methods described herein also include those that encode GBA and/or an M2-promoting agent downstream of a mammalian promoter. Promoters that are useful for the expression of GBA and/or an M2-promoting agent in mammalian cells include, e.g., elongation factor 1-alpha (EF1α) promoter, phosphoglycerate kinase 1 (PGK) promoter, CD68 molecule (CD68) promoter (see Dahl et al., Molecular Therapy 23:835 (2015), incorporated herein by reference as it pertains to the use of PGK and CD68 promoters to express GBA), C-X3-C motif chemokine receptor 1 (CX3CR1) promoter, integrin subunit alpha M (ITGAM) promoter, allograft inflammatory factor 1 (AIF1) promoter, purinergic receptor P2Y12 (P2Y12) promoter, transmembrane protein 119 (TMEM119) promoter, and colony stimulating factor 1 receptor (CSF1R) promoter. Alternatively, promoters derived from viral genomes can also be used for the stable expression of these agents in mammalian cells. Examples of functional viral promoters that can be used to promote mammalian expression of these agents are adenovirus late promoter, vaccinia virus 7.5K promoter, simian virus 40 (SV40) promoter, cytomegalovirus promoter, tk promoter of herpes simplex virus (HSV), mouse mammary tumor virus (MMTV) promoter, long terminal repeat (LTR) promoter of human immunodeficiency virus (HIV), promoter of moloney virus, Epstein barr virus (EBV), Rous sarcoma virus (RSV), and the cytomegalovirus (CMV) promoter.

Once a polynucleotide encoding GBA and/or an M2-promoting agent has been incorporated into the nuclear DNA of a mammalian cell, the transcription of this polynucleotide can be induced by methods known in the art. For example, expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms are tetracycline and doxycycline. These reagents are commercially available (Life Technologies, Carlsbad, Calif.) and can be administered to a mammalian cell in order to promote gene expression according to established protocols.

Other DNA sequence elements that may be included in polynucleotides for use in the compositions and methods described herein are enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode GBA and/or an M2-promoting agent and additionally include a mammalian enhancer sequence. Many enhancer sequences are now known from mammalian genes, and examples are enhancers from the genes that encode mammalian globin, elastase, albumin, α-fetoprotein, and insulin. Enhancers for use in the compositions and methods described herein also include those that are derived from the genetic material of a virus capable of infecting a eukaryotic cell. Examples are the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription are disclosed in Yaniv et al., Nature 297:17 (1982). An enhancer may be spliced into a vector containing a polynucleotide encoding a water-forming NADH oxidase, for example, at a position 5′ or 3′ to this gene. In a preferred orientation, the enhancer is positioned at the 5′ side of the promoter, which in turn is located 5′ relative to the polynucleotide encoding GBA and/or an M2-promoting agent.

Polynucleotides encoding GBA and/or an M2-promoting agent may include regulatory elements capable of turning gene expression on or off. These regulatory elements may include switches or destabilizing domains. Exemplary destabilizing domains are mutants of the human FK506- and rapamycin-binding protein (FKBP12), which confer instability to other proteins fused to these destabilizing domains. FKBP12 mutants include N-terminal mutants F15S, V24A, H25R, E60G, and L106P, and C-terminal mutants M66T, R71G, D100G, D100N, E102G, and K105I, as characterized in Banaszynski et al., Cell 126:995 (2006), the disclosure of which is incorporated herein by reference as it pertains to FKBP12 destabilizing domains. FKBP12 destabilizing domains promote protein degradation. The small molecule ligand Shield-1 (Shld1) can be used to stabilize FKBP12 mutant-containing proteins by protecting them from degradation. Other destabilizing domains that can be used to regulate expression of GBA or M2-promoting agents include mutants of the E. coli dihydrofolate reductase (ecDHFR) and mutants of the human estrogen receptor ligand binding domain (ERLBD), which confer instability resulting in degradation when fused to a protein of interest and can be stabilized by small molecule ligand trimethoprim (TMP), or by CMP8 or 4-hydroxytamoxifen (4OHT), respectively, as described in Iwamoto et al., Chem Biol. 17:981 (2010) and Miyazaki et al., J Am Chem Soc., 134:3942 (2012), the disclosures of each of which are incorporated herein by reference as they pertain to destabilization domain systems. Additional destabilizing peptide/stabilizing ligand systems that can be used in conjunction with the compositions and methods described herein so as to independently modulate the expression of one or more genes, such as a gene encoding GBA and a gene encoding an M2-promoting agent (for example, IL-25) are further described in WO 2014/164828, US 2005/0214738, U.S. Pat. No. 8,173,792, US 2012/0178168, US 2014/0255361, WO 2014/164828, WO 2015/152813, WO 2014/043189, WO 2014/089158, U.S. Pat. No. 8,106,191, US 2015/0307850, and US 2014/0220062, the disclosures of which are incorporated herein by reference as they pertain to the use of destabilizing domains and their corresponding ligands.

### Cell-Specific Gene Expression

Interfering RNA (RNAi) are widely used to knock down the expression of endogenous genes by delivering small interfering RNA (siRNA) into cells triggering the degradation of complementary mRNA. An additional application is to utilize the diversity of endogenous micro RNAs (miRNA) to negatively regulate the expression of exogenously introduced transgenes tagged with artificial miRNA target sequences. These miRNA target tagged transgenes can be negatively regulated according to the activity of a given miRNA which can be tissue, lineage, activation, or differentiation stage specific. These artificial miRNA target sequences (miRTs) can be recognized as targets by a specific miRNA thus inducing post-transcriptional gene silencing. While robust transgene expression in targeted cells can have beneficial therapeutic results, off target expression, such as the ectopic or non-regulated transgene expression in HSPCs or other progenitor cells, can have cytotoxic effects, which can result in counter-selection of transgene-containing cells leading to altered cellular behavior and reduced therapeutic efficacy. The incorporation of miRNA target sequences (miRTs) for miRNAs widely expressed in HSPCs and progenitors, but absent in cells of the myeloid lineage can allow for repressed transgene expression in HSPCs and other progenitor cells allowing for silent, long-term reservoir transgene-containing hematopoietic progeny, while allowing for robust transgene expression in differentiated, mature target cells. miR-126 is highly expressed in HSPCs, other progenitor cells, and cells of the erythroid lineage, but absent from those of the myeloid lineage (e.g., macrophages and microglia) (Gentner et al., Science Translational Medicine. 2:58ra34 (2010)). A miR-126 targeting sequence, for example, incorporated within a transgene would allow for targeted expression of the transgene in cells of the myeloid lineage and repressed expression in HSPCs and other progenitor cells, thus minimizing off-target cytotoxic effects. In some embodiments, the transgene encoding GBA and/or an M2-promoting agent may include a miR-126 targeting sequence.

### Signal Peptides

Polynucleotides encoding GBA may include one or more polynucleotides encoding a signal peptide. Signal peptides may have amino acid sequence of 5-30 residues in length, and may be located upstream of (e.g., 5′ to) a polynucleotide encoding GBA. These signal peptides allow for recognition of the nascent GBA polypeptides during synthesis by signal recognition particles resulting transport across the membrane of the rough endoplasmic reticulum, as well as glycosylation for transport into lysosomes. Exemplary signal peptides for lysosomal transport of GBA are those from GBA.

### Secretory Signal Peptides

Polynucleotides encoding GBA may include one or more polynucleotides encoding a secretory signal peptide. Secretory signal peptides may have amino acid sequences of 5-30 residues in length, and may be located upstream of (i.e., 5′ to) a polynucleotide encoding GBA. These secretory signal peptides allow for the recognition of the nascent polypeptides during synthesis by signal recognition particles resulting in translocation to the ER, packaging into transport vesicles, and finally, secretion. Exemplary secretory signal peptides for protein secretion are those from GBA, IGF-II, alpha-1 antitrypsin, IL-2, IL-6, CD5, immunoglobulins, trypsinogen, serum albumin, prolactin, elastin, tissue plasminogen activator signal peptide (tPA-SP), and insulin. In some embodiments, pluripotent cells (e.g., CD34+ cells) expressing a secreted form of GBA may be utilized as a therapeutic strategy to correct an enzyme deficiency (e.g., GBA) by infusing the missing enzyme into the bloodstream. As the blood perfuses patient tissues, GBA is taken up by cells and transported to the lysosome, where the enzyme acts to eliminate glucocerebroside that has accumulated in the lysosomes due to the enzyme deficiency. For lysosomal enzyme replacement therapy to be effective, the therapeutic enzyme (e.g., GBA) must be delivered to lysosomes in the appropriate cells in tissues where the storage defect is manifest. Conventional lysosomal enzyme replacement therapeutics are delivered using carbohydrates naturally attached to the protein to engage specific receptors on the surface of the target cells. One receptor, the cation-independent mannose-6-phosphate (M6P) receptor (CI-MPR), is particularly useful for targeting replacement lysosomal enzymes as the CI-MPR is present on the surface of most cell types.

### Glycosylation Independent Lysosomal Targeting

Glycosylation Independent Lysosomal Targeting (GILT) technology can be utilized to target therapeutic enzymes (e.g., GBA) to lysosomes. Specifically, the GILT technology uses a peptide tag instead of M6P to engage the CI-MPR for lysosomal targeting. Typically, a GILT tag is a protein, peptide, or other moiety that binds the CI-MPR in a mannose-6-phosphateindependent manner. Advantageously, this technology mimics the normal biological mechanism for uptake of lysosomal enzymes, yet does so in a manner independent of mannose-6-phosphate. In some embodiments, the GBA is secreted as a GBA fusion protein containing GBA and a GILT tag. In some embodiments, a GILT tag is derived from human insulin-like growth factor II (IGFII). Human IGF-II is a high affinity ligand for the CI-MPR; also referred to as IGF-II receptor. Binding of GILT-tagged therapeutic enzymes to the M6P/IGF-II receptor targets the protein to the lysosome via the endocytic pathway. A detailed description of GILT technology and the GILT tag can be found in U.S. Publication Nos. 20030082176, 20040006008, 20040005309, 20050281805, and 2009043207 the teachings of all of which are hereby incorporated by references in their entireties.

### Furin-Resistant GILT Tag

The IGF-II derived GILT tag may be subjected to proteolytic cleavage by furin during production in mammalian cells. Furin protease typically recognizes and cleaves a cleavage site having a consensus sequence Arg-X-X-Arg, where X is any amino acid. The cleavage site is positioned after the carboxy-terminal arginine (Arg) residue in the sequence. In some embodiments, a furin cleavage site has a consensus sequence Lys/Arg-X-X-X-Lys/Arg-Arg, where X is any amino acid. The cleavage site is positioned after the carboxy terminal arginine (Arg) residue in the sequence. The mature human IGF-II peptide sequence is shown below.

(SEQ ID NO. 12)
AYRPSETLCGGELVDTLQFVCGDRGFYFSRPASRVSRRSRGIVEECCFR
SCDLALLETYCATPAKSE

The mature human IGF-II contains two potential overlapping furin cleavage sites between residues 34-40 (bolded). Modified GILT tags that are resistant to cleavage by furin still retain ability to bind to the CI-MPR in a mannose-6-phosphate-independent manner. Specifically, furin-resistant GILT tags can be designed by mutating the amino acid sequence at one or more furin cleavage sites such that the mutation abolishes at least one furin cleavage site. Thus, in some embodiments, a furin-resistant GILT tag is a furin-resistant IGF-II mutein containing a mutation that abolishes at least one furin protease cleavage site or changes a sequence adjacent to the furin protease cleavage site such that the furin cleavage is prevented, inhibited, reduced or slowed down as compared to a wild-type IGF-II peptide (e.g., wild-type human mature IGF-II). A suitable mutation does not impact the ability of the furin-resistant GILT tag to bind to the human cation-independent mannose-6-phosphate receptor. In some embodiments, a furin-resistant IGF-II mutein suitable for use in conjunction with the compositions and methods described herein binds to the human cation-independent mannose-6-phosphate receptor in a mannose-6-phosphate-independent manner with a dissociation constant of 10⁻⁷ M or less (e.g., 10⁻⁸, 10⁻⁹, 10⁻¹⁰, 10⁻¹¹, or less) at pH 7.4. In some embodiments, a furin-resistant IGF-II mutein contains a mutation within a region corresponding to amino acids 30-40 (e.g., 31-40, 32-40, 33-40, 34-40, 30-39, 31-39, 32-39, 34-37, 32-39, 33-39, 34-39, 35-39, 36-39, 37-40, 34-40) of SEQ ID NO. 12. In some embodiments, a suitable mutation abolishes at least one furin protease cleavage site. A mutation can be amino acid substitutions, deletions, or insertions. For example, any one amino acid within the region corresponding to residues 30-40 (e.g., 31-40, 32-40, 33-40, 34-40, 30-39, 31-39, 32-39, 34-37, 32-39, 33-39, 34-39, 35-39, 36-39, 37-40, 34-40) of SEQ ID NO. 12 can be substituted with any other amino acid or deleted. For example, substitutions at position 34 may affect furin recognition of the first cleavage site. Insertion of one or more additional amino acids within each recognition site may abolish one or both furin cleavage sites. Deletion of one or more of the residues in the degenerate positions may also abolish both furin cleavage sites.

In some embodiments, a furin-resistant IGF-II mutein contains amino acid substitutions at positions corresponding to Arg37 or Arg40 of SEQ ID NO. 12. In some embodiments, a furin-resistant IGF-II mutein contains a Lys or Ala substitution at positions Arg37 or Arg40. Other substitutions are possible, including combinations of Lys and/or Ala mutations at both positions 37 and 40, or substitutions of amino acids other than Lys or Ala. In some embodiments, the furin-resistant IGF-II mutein suitable for use in conjunction with the compositions and methods described herein may contain additional mutations. For example, up to 30% or more of the residues of SEQ ID NO. 12 may be changed (e.g., up to 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% or more residues may be changed). Thus, a furin-resistant IGF-II mutein suitable for use in conjunction with the compositions and methods described herein may have an amino acid sequence at least 70%, including at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99%, identical to SEQ ID NO. 12. In some embodiments, a furin-resistant IGF-II mutein suitable for use in conjunction with the compositions and methods described herein is targeted specifically to the CI-MPR. Particularly useful are mutations in the IGF-II polypeptide that result in a protein that binds the CI-MPR with high affinity (e.g., with a dissociation constant of 10⁻⁷ M or less at pH 7.4) while binding other receptors known to be bound by IGF-II with reduced affinity relative to native IGF-II. For example, a furin-resistant IGF-II mutein suitable for use in conjunction with the compositions and methods described herein can be modified to have diminished binding affinity for the IGF-I receptor relative to the affinity of naturally-occurring human IGF-II for the IGF-I receptor. Additional mutational strategies have been utilized and are discussed at length in the U.S. Publication No. 2009043207, which is hereby incorporated by reference. For example, substitution of IGF-II residues Tyr 27 with Leu, Leu 43 with Val, or Ser 26 with Phe diminishes the affinity of IGF-II for the IGF-I receptor by 94-, 56-, and 4-fold respectively (Torres et al., J. Mol. Biol. 248(2):385-401 (1995)). Deletion of residues 1-7 of human IGF-II resulted in a 30-fold decrease in affinity for the human IGF-I receptor and a concomitant 12-fold increase in affinity for the rat IGF-II receptor (Hashimoto et al., J. Biol. Chem. 270(30):18013-8 (1995)). The NMR structure of IGF-II shows that Thr 7 is located near residues 48 Phe and 50 Ser, as well as near the 9 Cys-4 7 Cys disulfide bridge. It is thought that interaction of Thr 7 with these residues can stabilize the flexible N-terminal hexapeptide required for IGF-I receptor binding (Terasawa et al., EMBO J. 13(23)5590-7 (1994)). At the same time, this interaction can modulate binding to the IGF-II receptor. Truncation of the C-terminus of IGF-II (residues 62-67) also appears to lower the affinity of IGF-II for the IGF-I receptor by 5-old (Roth et al., Biochem. Biophys. Res. Commun. 181(2):907-14 (1991)). The binding surfaces for the IGF-I and cation-independent M6P receptors are on separate faces of IGF-II. Based on structural and mutational data, functional cation-independent M6P binding domains can be constructed that are substantially smaller than human IGF-II. For example, the amino terminal amino acids (e.g., 1-7 or 2-7) and/or the carboxy terminal residues 62-67 can be deleted or replaced. Additionally, amino acids 29-40 can likely be eliminated or replaced without altering the folding of the remainder of the polypeptide or binding to the cation-independent M6P receptor. Thus, a targeting moiety including amino acids 8-28 and 41-61 can be constructed. These stretches of amino acids could perhaps be joined directly or separated by a linker. Alternatively, amino acids 8-28 and 41-61 can be provided on separate polypeptide chains. Comparable domains of insulin, which are homologous to IGF-II and have a tertiary structure closely related to the structure of IGF-II, have sufficient structural information to permit proper refolding into the appropriate tertiary structure, even when present in separate polypeptide chains (Wang et al., Trends Biochem. Sci. 16(8):279-281 (1991)). Thus, for example, amino acids 8-28, or a conservative substitution variant thereof, could be fused to a lysosomal enzyme; the resulting fusion protein could be admixed with amino acids 41-61, or a conservative substitution variant thereof, and administered to a patient. IGF-II can also be modified to minimize binding to serum IGF-binding proteins (Baxter, Am. J. Physiol Endocrinol Metab. 278(6):967-76(2000)) to avoid sequestration of IGF-II/GILT constructs. A number of studies have localized residues in IGF-II necessary for binding to IGF-binding proteins. Constructs with mutations at these residues can be screened for retention of high affinity binding to the M6P/IGF-II receptor and for reduced affinity for IGF binding proteins. For example, replacing Phe 26 of IGF-II with Ser is reported to reduce affinity of IGF-II for IGFBP-1 and -6, with no effect on binding to the M6P/IGF-II receptor (Bach et al., J. Biol. Chem. 268(13):9246-54 (1993)). Other substitutions, such as Lys for Glu 9, can also be advantageous. The analogous mutations, separately or in combination, in a region of IGF-I that is highly conserved with IGF-II result in large decreases in IGF-BP binding (Magee et al., Biochemistry 38(48):15863-70 (1999)).

An alternate approach is to identify minimal regions of IGF-II that can bind with high affinity to the M6P/IGF-II receptor. The residues that have been implicated in IGF-II binding to the M6P/IGF-II receptor mostly cluster on one face of IGF-II (Terasawa et al., EMBO J. 13(23):5590-7 (1994)). Although IGF-II tertiary structure is normally maintained by three intramolecular disulfide bonds, a peptide incorporating the amino acid sequence on the M6P/IGF-II receptor binding surface of IGF-II can be designed to fold properly and have binding activity. Such a minimal binding peptide is a highly preferred lysosomal targeting domain. For example, a preferred lysosomal targeting domain is amino acids 8-67 of human IGF-II. Designed peptides, based on the region around amino acids 48-55, which bind to the M6P/IGF-II receptor, are also desirable lysosomal targeting domains. Alternatively, a random library of peptides can be screened for the ability to bind the M6P/IGF-II receptor either via a yeast two hybrid assay, or via a phage display type assay.

Many furin-resistant IGF-II muteins described herein have reduced or diminished binding affinity for the insulin receptor. Thus, in some embodiments, a peptide tag suitable for use in conjunction with the compositions and methods described herein has reduced or diminished binding affinity for the insulin receptor relative to the affinity of naturally occurring human IGF-II for the insulin receptor. In some embodiments, peptide tags with reduced or diminished binding affinity for the insulin receptor suitable for use in conjunction with the compositions and methods described herein include peptide tags having a binding affinity for the insulin receptor that is more than 1.5-fold, 2-fold, 3-fold, 4-fold, 5-fold, 6-fold, 7-fold, 8-fold, 9-fold, 10-fold, 12-fold, 14-fold, 16-fold, 18-fold, 20-fold, 50-fold, 100-fold less than that of the wild-type mature human IGF-II. The binding affinity for the insulin receptor can be measured using various in vitro and in vivo assays known in the art.

In some embodiments, the GILT tag has an amino acid sequence having at least 70% sequence identity (e.g., 70%, 75%, 80%, 85%, 90%, 95%, 96%, 97%, 98%, 99%, or greater, sequence identity) to the amino acid sequence of SEQ NO. 13, as shown below.

(SEQ ID NO. 13)
GGGGAGGGGAGGGGAGGGGAGGGPSLCGGELVDTLQFVCGDRGFYFSRP
ASRVSARSRGIVEECCFRSCDLALLETYCATPAKSE

In some embodiments, the GILT tag has an amino acid sequence having at least 70% sequence identity (e.g., 70%, 75%, 80%, 85%, 90%, 95%, 96%, 97%, 98%, 99%, or greater, sequence identity) to the amino acid sequence of SEQ NO. 14, as shown below.

(SEQ ID NO. 14)
GAPGGGSPAPAPTPAPAPTPAPAGGGPSGAPLCGGELVDTLQFVCGDRG
FYFSRPASRVSARSRGIVEECCFRSCDLALLETYCATPAKSE

In some embodiments, the GILT tag has an amino acid sequence having at least 70% sequence identity (e.g., 70%, 75%, 80%, 85%, 90%, 95%, 96%, 97%, 98%, 99%, or greater, sequence identity) to the amino acid sequence of SEQ NO. 15, as shown below.

(SEQ ID NO. 15)
GAPGGSPAGSPTSTEEGTSESATPESGPGTSTEPSEGSAPGSPAGSPTST
GPSGAPLCGGELVDTLQFVCGDRGFYFSRPASRVSARSRGIVEECCFRSC
DLALLETYCATPAKSE

In some embodiments, the GILT tag is encoded by a nucleic acid sequence having at least 85% sequence identity (e.g., 85%, 90%, 95%, 96%, 97%, 98%, 99%, or greater, sequence identity) to the nucleic acid sequence of SEQ ID NO. 17, as shown below.

(SEQ ID NO. 17)
GGCGGAGGCGGAGCTGGTGGCGGCGGAGCAGGCGGTGGTGGTGCAGGCGG
CGGAGGTGCTGGCGGAGGACCATCTCTTTGTGGCGGAGAACTGGTGGACA
CCCTGCAGTTCGTGTGTGGCGACAGAGGCTTCTACTTTAGCAGACCCGCC
AGCAGAGTGTCCGCCAGATCTAGAGGAATCGTGGAAGAGTGCTGCTTCAG
AAGCTGCGACCTGGCACTGCTGGAAACCTACTGTGCCACACCAGCCAAGA
GCGAGTGATG

In some embodiments, the GILT tag is encoded by a nucleic acid sequence having at least 85% sequence identity (e.g., 85%, 90%, 95%, 96%, 97%, 98%, 99%, or greater, sequence identity) to the nucleic acid sequence of SEQ ID NO. 18, as shown below.

(SEQ ID NO. 18)
GGAGCACCAGGCGGAGGATCTCCAGCTCCTGCTCCTACACCAGCTCCAG
CACCGACGCCTGCTCCAGCTGGCGGAGGACCTTCTGGTGCACCTCTTTG
TGGCGGAGAGCTGGTGGATACCCTGCAGTTCGTGTGTGGCGACCGGGGC
TTCTACTTTAGCAGACCTGCCAGCAGAGTGTCCGCCAGATCTAGAGGCA
TCGTGGAAGAGTGCTGCTTCAGAAGCTGCGACCTGGCACTGCTGGAAAC
CTACTGTGCCACACCAGCCAAGAGCGAGTGATGA

In some embodiments, the GILT tag is encoded by a nucleic acid sequence having at least 85% sequence identity (e.g., 85%, 90%, 95%, 96%, 97%, 98%, 99%, or greater, sequence identity) to the nucleic acid sequence of SEQ ID NO. 19, as shown below.

(SEQ ID NO. 19)
GGAGCACCAGGCGGATCTCCAGCAGGATCTCCAACCTCTACCGAGGAAG
GCACAAGCGAGTCTGCCACACCTGAGTCTGGACCTGGCACAAGCACAGA
GCCTAGCGAAGGATCTGCCCCAGGTTCTCCTGCCGGCTCTCCTACAAGT
ACAGGACCTTCTGGCGCTCCACTGTGTGGCGGAGAACTGGTGGATACCC
TGCAGTTCGTGTGCGGCGACAGAGGCTTCTACTTTAGCAGACCCGCCAG
CAGAGTGTCCGCCAGATCTAGAGGAATCGTGGAAGAGTGCTGCTTCAGA
AGCTGCGATCTGGCACTGCTGGAAACCTACTGTGCCACACCAGCCAAGA
GCGAGTGATGA

### ApoE Tag for BBB Penetrance of Secreted GBA

In some embodiments, the secreted GBA (e.g., secreted GBA fusion protein) is modified to penetrate the blood-brain barrier (BBB). Modifications for mediating BBB penetrance are well known in the art. Exemplary modifications are the use of tags containing the receptor binding (Rb) domain (amino acid residues 148-173 of SEQ ID NO. 21) of apolipoprotein E (ApoE). The complete ApoE peptide sequence is shown below.

(SEQ ID NO. 21)
MKVLWAALLVTFLAGCQAKVEQAVETEPEPELRQQTEWQSGQRWELALG
RFWDYLRWVQTLSEQVQEELLSSQVTQELRALMDETMKELKAYKSELEE
QLTPVAEETRARLSKELQAAQARLGADMEDVCGRLVQYRGEVQAMLGQS
TEELRVRLASHLRKLRKRLLRDADDLQKRLAVYQAGAREGAERGLSAIR
ERLGPLVEQGRVRAATVGSLAGQPLQERAQAWGERLRARMEEMGSRTRD
RLDEVKEQVAEVRAKLEEQAQQIRLQAEAFQARLKSWFEPLVEDMQRQW
AGLVEKVQAAVGTSAAPVPSDNH

ApoE is an important protein involved in lipid transport, and its cellular internalization is mediated by several members of the low density lipoprotein (LDL) receptor gene family, including the LDL receptor, very low-density lipoprotein receptor (VLDLR), and LDL receptor-related proteins (LRPs, including LRP1, LRP2, and LRP8). The LDL receptor is found to be highly expressed in brain capillary endothelial cells (BCECs), with down-regulated expression observed in peripheral vessels. Restricted expressions of LRPs and VLDLR have also been shown prominently in the liver and brain when they have been detected in BCECs, neurons, and glial cells. Several members of the low-density lipoprotein receptor family (LDLRf) proteins, including LRP1 and VLDLR, but not LDLR, are highly expressed in BBB-forming BCECs. These proteins can bind ApoE to facilitate their transcytosis into the abluminal side of the BBB.

In addition, receptor-associated protein (RAP), an antagonist as well as a ligand for both LRP1 and VLDLR, has been shown to have higher permeability across the BBB than transferrin in vivo and in vitro (Pan et al., J. Cell Sci. 117:5071-8 (2004)), indicating that these lipoprotein receptors (LDLRf) can represent efficient BBB delivery targets despite their lower expression than the transferrin receptor. As described herein, a potent receptor-binding peptide (Rb) derived from ApoE, has the ability to translocate protein across the BBB into the brain when engineered as fusion proteins. This method can therefore function to selectively open the BBB for therapeutic agents (e.g., soluble GBA) when engineered as a fusion protein. This peptide can be readily attached to diagnostic or therapeutic agents without jeopardizing their biological functions or interfering with the important biological functions of ApoE due to the utilization of the Rb domain of ApoE, rather than the entire ApoE protein. This pathway is also an alternative uptake pathway that can facilitate further/secondary distribution within the brain after the agents reach the CNS due to the widespread expression of LDLRf members in brain parenchyma. Regardless of application strategies, e.g., enzyme replacement therapy or cell-based, gene-based therapy, both the quantity and distribution of therapeutics within the brain parenchyma will have a significant impact on the clinical outcome of disease treatment. The development of and a detailed description of the use of the Rb domain of ApoE in targeted delivery of proteins across the BBB can be found in U.S. Publication No. 20140219974, which is hereby incorporated by reference in its entirety.

In some embodiments, the secreted GBA fusion protein has a peptide sequence containing the LDLRf receptor-binding domain (Rb) of SEQ ID NO. 21, or a fragment, variant, or oligomer thereof. An exemplary receptor-binding domain can be found in the N-terminus of ApoE, for example, between amino acid residues 1 to 191 of SEQ ID NO. 21, between amino acid residues 25 to 185 of SEQ ID NO. 21, between amino acid residues 50 to 180 of SEQ ID NO. 21, between amino acid residues 75 to 175 of SEQ ID NO. 21, between amino acid residues 100 to 170 of SEQ ID NO. 21, or between amino acid residues 125 to 165 of SEQ ID NO. 21. An exemplary receptor binding domain has the amino acid sequence of residues 159 to 167 of SEQ ID NO. 21.

In some embodiments, the peptide sequence containing the receptor-binding domain of ApoE can include at least one amino acid mutation, deletion, addition, or substitution. In some embodiments, the amino acid substitutions can be a combination of two or more mutations, deletions, additions, or substitutions. In some embodiments, the at least one substation is a conservative substitution. In some embodiments, the at least one amino acid addition includes addition of a selected sequence already found in the Rb domain of ApoE. A person of ordinary skill in the art will recognize suitable modifications that can be made to the sequence while retaining some degree of the biochemical activity for transport across the BBB.

### Vectors for the Expression of GBA and M2-Promoting Agents

In addition to achieving high rates of transcription and translation, stable expression of an exogenous gene in a mammalian cell can be achieved by integration of the polynucleotide containing the gene into the nuclear genome of the mammalian cell. A variety of vectors for the delivery and integration of polynucleotides encoding exogenous proteins into the nuclear DNA of a mammalian cell have been developed. Examples of expression vectors are disclosed in, e.g., WO 1994/011026 and are incorporated herein by reference. Expression vectors for use in the compositions and methods described herein contain a polynucleotide sequence that encodes GBA and/or an M2-promoting agent, as well as, e.g., additional sequence elements used for the expression of these agents and/or the integration of these polynucleotide sequences into the genome of a mammalian cell. Certain vectors that can be used for the expression of GBA and M2-promoting agents include plasmids that contain regulatory sequences, such as promoter and enhancer regions, which direct gene transcription. Other useful vectors for expression of GBA and M2-promoting agents contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin.

Expression vectors for use in the compositions and methods described herein may express GBA and one or more M2-promoting agents from monocistronic or polycistronic expression cassettes. A monocistronic expression cassette contains a polynucleotide sequence that encodes a single gene. Pluripotent cells (e.g., CD34+ cells) described herein can be transfected with multiple plasmids, for example, each containing a monocistronic expression cassette, or with a single plasmid containing more than one monocistronic expression cassette. Polycistronic expression cassettes can be used to simultaneously express two or more proteins from a single transcript. Polycistronic expression cassettes include bicistronic expression cassettes, which can be used to generate two proteins from a single transcript and may include IRES sequences to recruit ribosomes to initiate translation from a region of the mRNA other than the 5′ cap.

Foot-and-mouth disease virus 2A (FMDV 2A) polynucleotides can be utilized to express two or more genes (e.g., 2 genes, 3 genes, 4 genes, 5 genes or more), and can be used in bicistronic or polycistronic expression cassettes to produce equimolar levels of multiple genes from the same transcript. FMDV 2A mediates a cotranslational cleavage event, which separates proteins linked by 2A sequences, and multiple 2A sequences may be used in one vector. For an example of the use of FMDV 2A to express multiple proteins, see Ryan and Drew, EMBO Journal 13:928 (1994), the disclosure of which is incorporated herein by reference as it pertains to the use of FMDV 2A sequences. 2A-like sequences from other viruses can also be used in the compositions and methods described herein, including the 2A-like sequences from equine rhinitis A virus (E2A), porcine teschovirus-1 (P2A), and Thosea asigna virus (T2A), as described in Szymczak and Vignali, Expert Opin Biol Ther. 5:627 (2005), Szymczak et al., Nat Biotechnol. 22:589 (2004), and Osborn et al., Molecular Therapy 12:569 (2005), the disclosures of which are incorporated herein by reference as they pertain to the use of 2A-like sequences in gene expression.

### Viral Vectors for Expression of GBA and M2-Promoting Agents

Viral genomes provide a rich source of vectors that can be used for the efficient delivery of exogenous genes into a mammalian cell. Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors are a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses are: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, (1996))). Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030), the teachings of which are incorporated herein by reference.

### AA V Vectors for Nucleic Acid Delivery

Nucleic acids of the compositions and methods described herein may be incorporated into rAAV vectors and/or virions in order to facilitate their introduction into a cell. AAV vectors can be used in the central nervous system, and appropriate promoters and serotypes are discussed in Pignataro et al., J Neural Transm (2017), epub ahead of print, the disclosure of which is incorporated herein by reference as it pertains to promoters and AAV serotypes useful in CNS gene therapy. rAAV vectors useful in the compositions and methods described herein are recombinant nucleic acid constructs that include (1) a heterologous sequence to be expressed (e.g., a polynucleotide encoding GBA and/or an M2-promoting agent) and (2) viral sequences that facilitate integration and expression of the heterologous genes. The viral sequences may include those sequences of AAV that are required in cis for replication and packaging (e.g., functional ITRs) of the DNA into a virion. Such rAAV vectors may also contain marker or reporter genes. Useful rAAV vectors have one or more of the AAV WT genes deleted in whole or in part, but retain functional flanking ITR sequences. The AAV ITRs may be of any serotype suitable for a particular application. Methods for using rAAV vectors are described, for example, in Tal et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2, and VP3, which are required for virion assembly. The construction of rAAV virions has been described, for example, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8, 9, 10 and rh74. For targeting cells located in or delivered to the central nervous system, AAV2, AAV9, and AAV10 may be particularly useful. Construction and use of AAV vectors and AAV proteins of different serotypes are described, for example, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype pseudotyped with a capsid gene derived from a serotype other than the given serotype (e.g., AAV1, AAV2, AAV3, AAV4, AAV5, AAV6, AAV7, AAV8, AAV9, and AAV10, among others). Techniques involving the construction and use of pseudotyped rAAV virions are known in the art and are described, for example, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

### Retroviral Vectors

The delivery vector used in the methods and compositions described herein may be a retroviral vector. One type of retroviral vector that may be used in the methods and compositions described herein is a lentiviral vector. Lentiviral vectors (LVs), a subset of retroviruses, transduce a wide range of dividing and non-dividing cell types with high efficiency, conferring stable, long-term expression of the transgene. An overview of optimization strategies for lentiviral vectors is provided in Delenda, The Journal of Gene Medicine 6: S125 (2004), the disclosure of which is incorporated herein by reference.

The use of lentivirus-based gene transfer techniques relies on the in vitro production of recombinant lentiviral particles carrying a highly deleted viral genome in which the transgene of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, incapsidation, and expression, in which the sequences to be expressed are inserted.

A lentiviral vector used in the methods and compositions described herein may include one or more of a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating LTR (SIN-LTR). The lentiviral vector optionally includes a central polypurine tract (cPPT) and a woodchuck hepatitis virus post-transcriptional regulatory element (WPRE), as described in U.S. Pat. No. 6,136,597, the disclosure of which is incorporated herein by reference as it pertains to WPRE. The lentiviral vector may further include a pHR′ backbone, which may include for example as provided below.

The Lentigen lentiviral vector described in Lu et al., Journal of Gene Medicine 6:963 (2004) may be used to express the DNA molecules and/or transduce cells.

A lentiviral vector used in the methods and compositions described herein may a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating LTR (SIN-LTR). It will be readily apparent to one skilled in the art that optionally one or more of these regions is substituted with another region performing a similar function.

GBA or the M2-promoting agent is required to be expressed at sufficiently high levels. Transgene expression is driven by a promoter sequence. Optionally, the lentiviral vector includes a CMV promoter. The promoter may also be EF1α or PGK promoter. In another embodiment, the promoter is a microglia-specific promoter, e.g., CD68 promoter, CX3CR1 promoter, ITGAM promoter, AIF1promoter, P2Y12 promoter, TMEM119 promoter, or CSF1R promoter. A person skilled in the art will be familiar with a number of promoters that will be suitable in the vector constructs described herein.

Enhancer elements can be used to increase expression of modified DNA molecules or increase the lentiviral integration efficiency. The lentiviral vector used in the methods and compositions described herein may include a nef sequence. The lentiviral vector used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The lentiviral vector used in the methods and compositions described herein may include a Woodchuck Posttranscriptional Regulatory Element (W PRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to lentiviral vector results in a substantial improvement in the level of transgene expression from several different promoters, both in vitro and in vivo. The lentiviral vector used in the methods and compositions described herein may include both a cPPT sequence and WPRE sequence. The vector may also include an internal ribosome entry site (IRES) sequence that permits the expression of multiple polypeptides from a single promoter.

In addition to IRES sequences, other elements which permit expression of multiple polypeptides are useful. The vector used in the methods and compositions described herein may include multiple promoters that permit expression more than one polypeptide. The vector used in the methods and compositions described herein may include a protein cleavage site that allows expression of more than one polypeptide. Examples of protein cleavage sites that allow expression of more than one polypeptide are described in Klump et al., Gene Ther.; 8:811 (2001), Osborn et al., Molecular Therapy 12:569 (2005), Szymczak and, Vignali,. Expert Opin Biol Ther. 5:627 (2005), and Szymczak et al., Nat Biotechnol. 22:589 (2004), the disclosures of which are incorporated herein by reference as they pertain to protein cleavage sites that allow expression of more than one polypeptide. It will be readily apparent to one skilled in the art that other elements that permit expression of multiple polypeptides identified in the future are useful and may be utilized in the vectors suitable for use with the compositions and methods described herein.

The vector used in the methods and compositions described herein may, be a clinical grade vector.

### Viral Regulatory Elements

The viral regulatory elements are components of delivery vehicles used to introduce nucleic acid molecules into a host cell. The viral regulatory elements are optionally retroviral regulatory elements. For example, the viral regulatory elements may be the LTR and gag sequences from HSC1 or MSCV. The retroviral regulatory elements may be from lentiviruses or they may be heterologous sequences identified from other genomic regions.

One skilled in the art would also appreciate that as other viral regulatory elements are identified, these may be used with the nucleic acid molecules described herein.

### Methods for the Delivery of Exogenous Nucleic Acids to Target Cells

Techniques that can be used to introduce a polynucleotide, such as codon-optimized DNA or RNA (e.g., mRNA, tRNA, siRNA, miRNA, shRNA, chemically modified RNA) into a mammalian cell are well known in the art. For example, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids. Electroporation of mammalian cells is described in detail, e.g., in Chu et al., Nucleic Acids Research 15:1311 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Experimental Dermatology 14:315 (2005), as well as in US 2010/0317114, the disclosures of each of which are incorporated herein by reference.

Additional techniques useful for the transfection of target cells are the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., Journal of Visualized Experiments 81:e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386, the disclosure of which is incorporated herein by reference. Similar techniques that exploit ionic interactions with the cell membrane to provoke the uptake of foreign nucleic acids are contacting a cell with a cationic polymer-nucleic acid complex. Exemplary cationic molecules that associate with polynucleotides so as to impart a positive charge favorable for interaction with the cell membrane are activated dendrimers (described, e.g., in Dennig, Topics in Current Chemistry 228:227 (2003), the disclosure of which is incorporated herein by reference) polyethylenimine, and diethylaminoethyl (DEAE)-dextran, the use of which as a transfection agent is described in detail, for example, in Gulick et al., Current Protocols in Molecular Biology 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for example, in US 2010/0227406, the disclosure of which is incorporated herein by reference.

Another useful tool for inducing the uptake of exogenous nucleic acids by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107: 1870 (2010), the disclosure of which is incorporated herein by reference.

Magnetofection can also be used to deliver nucleic acids to target cells. The magnetofection principle is to associate nucleic acids with cationic magnetic nanoparticles. The magnetic nanoparticles are made of iron oxide, which is fully biodegradable, and coated with specific cationic proprietary molecules varying upon the applications. Their association with the gene vectors (DNA, siRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Therapy 9:102 (2002), the disclosure of which is incorporated herein by reference.

Another useful tool for inducing the uptake of exogenous nucleic acids by target cells is sonoporation, a technique that involves the use of sound (typically ultrasonic frequencies) for modifying the permeability of the cell plasma membrane permeabilize the cells and allow polynucleotides to penetrate the cell membrane. This technique is described in detail, e.g., in Rhodes et al., Methods in Cell Biology 82:309 (2007), the disclosure of which is incorporated herein by reference.

Microvesicles represent another potential vehicle that can be used to modify the genome of a target cell according to the methods described herein. For example, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Modulation of Gene Expression Using Gene Editing Techniques

### Disruption of Endogenous GBA

In some embodiments, endogenous GBA is disrupted (e.g., in a subject undergoing treatment, such as in a population of neurons in a subject undergoing treatment, or in the pluripotent cells to be administered to the subject). Exemplary methods for disrupting endogenous GBA expression are those in which an inhibitory RNA molecule is administered to the subject, or contacted with a population of neurons in the subject or the population of pluripotent cells to be administered to the subject. The inhibitory RNA molecule may function to disrupt endogenous GBA expression, for example, act by way of the RNA interference (RNAi) pathway. An inhibitory RNA molecule can decrease the expression level (e.g., protein level or mRNA level) of endogenous GBA. For example, an inhibitory RNA molecule includes a short interfering RNA, short hairpin RNA, and/or a microRNA that targets full-length endogenous GBA. A siRNA is a double-stranded RNA molecule that typically has a length of about 19-25 base pairs. A shRNA is a RNA molecule including a hairpin turn that decreases expression of target genes via RNAi. shRNAs can be delivered to cells in the form of plasmids, e.g., viral or bacterial vectors, e.g., by transfection, electroporation, or transduction). A microRNA is a non-coding RNA molecule that typically has a length of about 22 nucleotides. miRNAs bind to target sites on mRNA molecules and silence the mRNA, e.g., by causing cleavage of the mRNA, destabilization of the mRNA, or inhibition of translation of the mRNA. An inhibitory RNA molecule can be modified, e.g., to contain modified nucleotides, e.g., 2′-fluoro, 2′-o-methyl, 2′-deoxy, unlocked nucleic acid, 2′-hydroxy, phosphorothioate, 2′-thiouridine, 4′-thiouridine, 2′-deoxyuridine. Without being bound by theory, it is believed that certain modification can increase nuclease resistance and/or serum stability, or decrease immunogenicity.

In some embodiments, the inhibitory RNA molecule decreases the level and/or activity or function of endogenous GBA. In embodiments, the inhibitory RNA molecule inhibits expression of endogenous GBA. In other embodiments, the inhibitor RNA molecule increases degradation of endogenous GBA and/or decreases the stability of endogenous GBA. The inhibitory RNA molecule can be chemically synthesized or transcribed in vitro.

In some embodiments, the endogenous GBA is disrupted in the pluripotent stem cells containing the GBA transgene using, for example, the gene editing techniques described herein. In some embodiments, the endogenous GBA is globally disrupted in the subject using, for example, the gene editing techniques described herein. In some embodiments, the endogenous GBA is disrupted in a population of neurons in the subject using, for example, the gene editing techniques described herein. In some embodiments, disruption of endogenous GBA in the subject, neurons, and/or pluripotent cells containing the GBA transgene occurs prior to administration of the pluripotent stem cells to the subject.

The making and use of inhibitory therapeutic agents based on non-coding RNA such as ribozymes, RNAse P, siRNAs, and miRNAs are also known in the art, for example, as described in Sioud, RNA Therapeutics: Function, Design, and Delivery (Methods in Molecular Biology). Humana Press 2010.

### Nuclease-Mediated Gene Transfer

Another useful tool for the disruption and integration of target genes into the genome of a pluripotent stem cell is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and an associated Cas9 nuclease. This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas9 nuclease to this site. In this manner, highly site-specific Cas9-mediated DNA cleavage can be engendered in a foreign polynucleotide because the interaction that brings Cas9 within close proximity of the target DNA molecule is governed by RNA: DNA hybridization. As a result, one can theoretically design a CRISPR/Cas system to cleave any target DNA molecule of interest (e.g., endogenous GBA). This technique has been exploited in order to edit eukaryotic genomes (Hwang et al. Nature Biotechnology 31:227 (2013), the disclosure of which is incorporated herein by reference) and can be used as an efficient means of site-specifically editing pluripotent stem cell genomes in order to cleave DNA prior to the incorporation of a gene encoding a target gene. The use of CRISPR/Cas to modulate gene expression has been described in, e.g., U.S. Pat. No. 8,697,359, the disclosure of which is incorporated herein by reference. Alternative methods for disruption of a target DNS by site-specifically cleaving genomic DNA prior to the incorporation of a gene of interest in a pluripotent stem cell include the use of zinc finger nucleases (ZFNs) and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. The use of ZFNs and TALENs in genome editing applications is described, e.g., in Urnov et al. Nature Reviews Genetics 11:636 (2010); and in Joung et al. Nature Reviews Molecular Cell Biology 14:49 (2013), the disclosure of both of which are incorporated herein by reference. In some embodiments, the endogenous GBA may be disrupted in the pluripotent stem cells containing the GBA transgene and/or M2-promoting agents using these gene editing techniques described herein.

### Transposon-Mediated Gene Transfer

In addition to viral vectors, a variety of additional tools have been developed that can be used for the incorporation of exogenous genes into pluripotent stem cells (e.g., CD34+ cells). One such method that can be used for incorporating polynucleotides encoding target genes into pluripotent stem cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In certain cases, these excision sites may be terminal repeats or inverted terminal repeats. Once excised from the transposon, the gene of interest can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the target gene is first transcribed to an RNA product and then reverse-transcribed to DNA before incorporation in the mammalian cell genome. Transposon systems include the piggybac transposon (described in detail in, e.g., WO 2010/085699) and the sleeping beauty transposon (described in detail in, e.g., US2005/0112764), the disclosures of each of which are incorporated herein by reference.

### Methods of Treatment

### Selection of Subjects

Subjects that may be treated as described herein are subjects having or at risk of developing Parkinson's disease. The type of PD may be GBA-associated PD, sporadic PD, PD caused by an environmental toxin, e.g., herbicides or pesticides, or PD associated with a non-GBA mutation, e.g., a mutation in one or more of the genes encoding parkin, PINK1, LRRK2, or DJ-1. The compositions and methods described herein can be used to treat patients with normal GBA activity, reduced GBA activity, and patients whose GBA mutational status and/or GBA activity level is unknown. The compositions and methods described herein may also be administered as a preventative treatment to patients at risk of developing Parkinson's disease, e.g., patients with a GBA mutation, patients with reduced GBA activity, patients with a mutation in one or more of the genes encoding parkin, PINK1, LRRK2, or DJ-1, or patients exposed to an environmental toxin associated with PD. Patients at risk for PD may show early symptoms of PD or may not yet be symptomatic when treatment is administered.

### Routes of Administration

The cells and compositions described herein may be administered to a subject with Parkinson's disease by a variety of routes, such as intracerebroventricularly, stereotactically, intravenously, intraosseously, or by means of a bone marrow transplant. In some embodiments, the cells and compositions described herein may be administered to a subject systemically (e.g., intravenously), directly to the central nervous system (CNS) (e.g., intracerebroventricularly or stereotactically), or directly into the bone marrow (e.g., intraosseously). In some embodiments, the cells and compositions described herein are administered to a subject intracerebroventricularly into the cerebral lateral ventricles (a description of this method can be found in Capotondo et al., Science Advances 3:e1701211 (2017), incorporated herein by reference as it pertains to intracerebroventricular injection of hematopoietic stem and progenitor cells into the cerebral lateral ventricles of mouse models). In some embodiments, the cells and compositions described herein are administered to a subject by stereotactic injection into the substantia nigra (a description of this method can be found in Altarche-Xifro et al., EBiomedicine 8:83 (2016), incorporated herein by reference as it pertains to stereotactic injection of hematopoietic stem and progenitor cells into the substantia nigra of PD mouse models). The most suitable route for administration in any given case will depend on the particular cell or composition administered, the patient, pharmaceutical formulation methods, administration methods (e.g., administration time and administration route), the patient's age, body weight, sex, severity of the diseases being treated, the patient's diet, and the patient's excretion rate. Multiple routes of administration may be used to treat a single subject, e.g., intracerebroventricular or stereotactic injection and intravenous injection, intracerebroventricular or stereotactic injection and intraosseous injection, or intracerebroventricular or stereotactic injection and bone marrow transplant. Multiple routes of administration may be used to treat a single subject at one time, or the subject may receive treatment via one route of administration first, and receive treatment via another route of administration during a second appointment, e.g., 1 week later, 2 weeks later, 1 month later, 6 months later, or 1 year later. Cells may be administered to a subject once, or cells may be administered one or more times (e.g., 2-10 times) per week, month, or year to a subject treatment for Parkinson's disease.

### Conditioning

Prior to administration of cells or compositions, it may be advantageous to deplete or ablate endogenous microglia and/or hematopoietic stem and progenitor cells. Microglia and/or hematopoietic stem and progenitor cells can be ablated through the use of chemical agents (e.g., busulfan, treosulfan, PLX3397, PLX647, PLX5622, or clodronate liposomes), irradiation, or a combination thereof. The agents used for cell ablation may be BBB penetrating (e.g., busulfan) or may lack the ability to cross the BBB (e.g., treosulfan). Exemplary microglia and/or hematopoietic stem and progenitor cells ablating agents are busulfan (Capotondo et al., PNAS 109:15018 (2012), the disclosure of which is incorporated by reference as it pertains to the use of busulfan to ablate microglia), treosulfan, PLX3397, PLX647, PLX5622, or clodronate liposomes. Other agents for the depletion of endogenous microglia and/or hematopoietic stem and progenitor cells include cytotoxins covalently conjugated to antibodies or antigen binding fragments thereof capable of binding antigens expressed by hematopoietic stem cells so as to form an antibody-drug conjugate. Cytotoxins suitable for antibody drug conjugates include DNA-intercalating agents, (e.g., anthracyclines), agents capable of disrupting the mitotic spindle apparatus (e.g., vinca alkaloids, maytansine, maytansinoids, and derivatives thereof), RNA polymerase inhibitors (e.g., an amatoxin, such as α-amanitin and derivatives thereof), agents capable of disrupting protein biosynthesis (e.g., agents that exhibit rRNA N-glycosidase activity, such as saporin and ricin A-chain), among others known in the art. Ablation may eliminate all microglia and/or hematopoietic stem and progenitor cells, or it may reduce microglia and/or hematopoietic stem and progenitor cells numbers by at least 10% (e.g., at least 20%, 30%, 40%, 50%, 60%, 70%, 80%, 90%, 95%, or more). One or more agents to ablate microglia and/or hematopoietic stem and progenitor cells may be administered at least one week (e.g., 1, 2, 3, 4, 5, or 6 weeks or more) before administration of the cells or compositions described herein. Cells administered in the methods described herein may replace the ablated microglia and/or hematopoietic stem and progenitor cells, and may repopulate the brain following intracerebroventricular, stereotactic, intravenous, or intraosseous injection, or following bone marrow transplant. Cells administered intravenously, intraosseously, or by bone marrow transplant may cross the blood brain barrier to enter the brain and differentiate into microglia. Cells administered to the brain, e.g., cells administered intracerebroventricularly or stereotactically, can differentiate into microglia in vivo or can be differentiated into microglia ex vivo.

### Stem Cell Rescue

The methods described herein may include administering to a subject a population of CD34+ cells (e.g., embryonic stem cells, induced pluripotent stem cells, hematopoietic stem cells, and myeloid progenitor cells). These cells may be CD34+ cells that have not been modified to express the transgene encoding GBA. These cells may have disrupted endogenous GBA. The CD34+ cells may be administered systemically (e.g., intravenously), or by bone marrow transplantation to reconstitute the bone marrow compartment following conditioning as described herein. For example, these CD34+ cells may migrate to a stem cell niche and increase the quantity of cells of the hematopoietic lineage at such a site by, for example, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or more. Administration may occur prior to, or following administration of the composition of the described herein.

### Selection of Donor Cells

In some embodiments, the subject is the donor. In such cases, withdrawn hematopoietic stem or progenitor cells may be re-infused into the subject (following modification (e.g., incorporation of the transgene encoding GBA and/or one or more M2-promoting agents, and/or disruption of endogenous GBA), such that the cells may subsequently home to hematopoietic tissue and establish productive hematopoiesis, thereby populating or repopulating a line of cells that is defective or deficient in the subject (e.g., a population of microglia). In this scenario, the transplanted hematopoietic stem or progenitor cells are least likely to undergo graft rejection, as the infused cells are derived from the patient and express the same HLA class I and class II antigens as expressed by the patient.

Alternatively, the subject and the donor may be distinct. In some embodiments, the subject and the donor are related, and may, for example, be HLA-matched. As described herein, HLA-matched donor-recipient pairs have a decreased risk of graft rejection, as endogenous T cells and NK cells within the transplant recipient are less likely to recognize the incoming hematopoietic stem or progenitor cell graft as foreign, and are thus less likely to mount an immune response against the transplant. Exemplary HLA-matched donor-recipient pairs are donors and recipients that are genetically related, such as familial donor-recipient pairs (e.g., sibling donor-recipient pairs).

In some embodiments, the subject and the donor are HLA-mismatched, which occurs when at least one HLA antigen, in particular with respect to HLA-A, HLA-B and HLA-DR, is mismatched between the donor and recipient. To reduce the likelihood of graft rejection, for example, one haplotype may be matched between the donor and recipient, and the other may be mismatched.

### Pharmaceutical Compositions and Dosing

The number of cells administered to a subject for the treatment of Parkinson's disease (e.g., GBA-associated Parkinson's disease) as described herein may depend, for example, on the expression level of GBA and, optionally, the expression level of the M2-promoting agent in the cells, the patient, pharmaceutical formulation methods, administration methods (e.g., administration time and administration route), the patient's age, body weight, sex, severity of the disease being treated, and whether or not the patient has been treated with agents to ablate endogenous microglia. The number of cells administered may be, for example, 1×10⁶ cells/kg to 1×10¹² cells/kg, or more (e.g., 1×10⁷ cells/kg, 1×10⁸ cells/kg, 1×10⁹ cells/kg, 1×10¹⁰ cells/kg, 1×10¹¹ cells/kg, 1×10¹² cells/kg, or more). Cells may be administered in an undifferentiated state, or after partial or complete differentiation into microglia. The number of CD34+ cells may be administered in any suitable dosage following conditioning. Non-limiting examples of dosages are about 1×10⁵ CD34+ cells/kg of recipient to about 1×10⁷ CD34+ cells/kg (e.g., from about 2×10⁵ CD34+ cells/kg to about 9×10⁶ CD34+ cells/kg, from about 3×10⁵ CD34+ cells/kg to about 8×10⁶ CD34+ cells/kg, from about 4×10⁵ CD34+ cells/kg to about 7×10⁶ CD34+ cells/kg, from about 5×10⁵ CD34+ cells/kg to about 6×10⁶ CD34+ cells/kg, from about 5×10⁵ CD34+ cells/kg to about 1×10⁷ CD34+ cells/kg, from about 6×10⁵ CD34+ cells/kg to about 1×10⁷ CD34+ cells/kg, from about 7×10⁵ CD34+ cells/kg to about 1×10⁷ CD34+ cells/kg, from about 8×10⁵ CD34+ cells/kg to about 1×10⁷ CD34+ cells/kg, from about 9×10⁵ CD34+ cells/kg to about 1×10⁷ CD34+ cells/kg, and from about 1×10⁶ CD34+ cells/kg to about 1×10⁷ CD34+ cells/kg). Additional exemplary dosages are from about 1×10¹⁰ CD34+ cells/kg of recipient to about 1×10¹² CD34+ cells/kg (e.g., from about 2×10¹⁰ CD34+ cells/kg to about 9×10¹¹ CD34+ cells/kg, from about 3×10¹⁰ CD34+ cells/kg to about 8×10¹¹ CD34+ cells/kg, from about 4×10¹⁰ CD34+ cells/kg to about 7×10¹¹ CD34+ cells/kg, from about 5×10¹⁰ CD34+ cells/kg to about 6×10¹¹ CD34+ cells/kg, from about 5×10¹⁰ CD34+ cells/kg to about 1×10¹² CD34+ cells/kg, from about 6×10¹⁰ CD34+ cells/kg to about 1×10¹² CD34+ cells/kg, from about 7×10¹⁰ CD34+ cells/kg to about 1×10¹² CD34+ cells/kg, from about 8×10¹⁰ CD34+ cells/kg to about 1×10¹² CD34+ cells/kg, from about 9×10¹⁰ CD34+ cells/kg to about 1×10¹² CD34+ cells/kg, and from about 1×10¹¹ CD34+ cells/kg to about 1×10¹² CD34+ cells/kg), among others.

The cells and compositions described herein can be administered in an amount sufficient to improve one or more pathological features in PD. Administration of the cells or compositions described herein may increase the quantity of M2 microglia in the brain of the subject relative to the quantity of M1 microglia in the brain of the subject, decrease the level of proinflammatory cytokines in the brain of the subject, increase the level of anti-inflammatory cytokines in the brain of the subject, improve the cognitive performance of the subject, improve the motor function of the subject, reduce α-synuclein levels or aggregation thereof in the subject, and/or reduce dopaminergic neuron loss in the subject. The numbers of M1 and M2 microglia may be assessed using ELISAs to compare the level of cytokines, chemokines, and other pro- and anti-inflammatory mediators in the cerebrospinal fluid (CSF) of subjects before and after treatment, by using PET imaging to view translocator protein (TSPO), a protein highly expressed in classically activated M1 microglia, before and after treatment, e.g., using TSPO radioligand ¹¹C-(R)-PK11195, or by analyzing the levels of M1- and M2-associated genes and proteins in a tissue sample using standard techniques, e.g., western blot analysis, immunohistochemical analyses, or quantitative RT-PCR. Cognition and motor function can be assessed using standard neurological tests before and after treatment, and monomeric and oligomeric α-synuclein can be detected in plasma and CSF using ELISA. Dopaminergic neuron loss can be assessed using F¹⁸-dopa PET scans or dopamine transporter imaging scans (123I-FP-CIT DaTSCANs). M2-promoting agents may reduce one or more pro-inflammatory cytokines, e.g., IL-1β, tumor necrosis factor, iNOS, IL-6, or IL-12, chemokines, M1-associated gene and/or protein levels, e.g., MHC II, CD86, CD68, CD11B, or Fcγ receptors, oligomeric α-synuclein levels, and/or TSPO signal by 10%, 15%, 20%, 25%, 30%, 35%, 40%, 45%, 50%, 55%, 60%, 65%, 70%, 75%, 80%, 85%, 90%, 95%, 100%, 200%, 500%, or more. M2-promoting agents may increase one or more anti-inflammatory cytokines, e.g., IL-25, IL-10 or TGF-β, chemokines, M2-associated gene and/or protein levels, e.g., CD163, TREM2, Arg1, or CD206, cognitive function, motor function, and/or F¹⁸-dopa signal by 10%, 15%, 20%, 25%, 30%, 35%, 40%, 45%, 50%, 55%, 60%, 65%, 70%, 75%, 80%, 85%, 90%, 95%, 100%, 200%, 500%, or more. M2-promoting agents may also slow or prevent a decrease in cognitive function, motor function, and/or F¹⁸-dopa signal. These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of pluripotent cells expressing one or more M2-promoting agents. The patient may be evaluated 1 month, 2 months, 3 months, 4 months, 5 months, 6 months or more following administration of the population of pluripotent cells depending on the route of administration used for treatment. Depending on the outcome of the evaluation, the patient may receive additional treatments.

### Kits

The compositions described herein can be provided in a kit for use in treating Parkinson's disease. Compositions may include host cells described herein (e.g., HSCs, iPSCs, ES cells, CD34+ cells, myeloid progenitor cells) that express GBA and one or more M2-promoting agents, and, optionally, may have disrupted endogenous GBA. Cells may be cryopreserved, e.g., in dimethyl sulfoxide (DMSO), glycerol, or another cryoprotectant. The kit can include a package insert that instructs a user of the kit, such as a physician, to perform the methods described herein. The kit may optionally include a syringe or other device for administering the composition.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Generation of a CD34+ Hematopoietic Stem Cell Expressing Non-Secreted Glucocerebrosidase (GBA) and, Optionally, an M2-Promoting Agent

An exemplary method for making pluripotent cells (e.g., CD34+ cells) that express non-secreted GBA and, optionally, an M2-promoting agent for use in the compositions and methods described herein is by way of transduction. Retroviral vectors (e.g., a lentiviral vector, alpharetroviral vector, or gammaretroviral vector) containing a microglia-specific promoter, such as the CD68 promoter, a signal peptide, such as the GBA signal peptide, and the polynucleotide encoding GBA can be engineered using standard techniques known in the art. If pluripotent cells that express GBA and an M2-promoting agent, such as IL-25, are to be made, a bicistronic expression cassette can be used in which an IRES sequence is placed between the polynucleotide encoding GBA and the polynucleotide encoding IL-25. After the retroviral vector is engineered, the retrovirus can be used to transduce pluripotent cells (e.g., iPS cells, ES cells, CD34+ HSCs) to generate a population of pluripotent cells that express non-secreted GBA and, optionally, an M2-promoting agent.

Additional exemplary methods for making pluripotent cells that express non-secreted GBA and, optionally, an M2-promoting agent for use in the compositions and methods described herein is transfection. Using molecular biology techniques known in the art, plasmid DNA containing a promoter, such as a microglia-specific promoter, (e.g., the CD68 promoter), a signal peptide, such as the GBA signal peptide, and the polynucleotide encoding GBA can be produced. For example, the GBA gene may be amplified from a human cell line using PCR-based techniques known in the art, or the gene may be synthesized, for example, using solid-phase polynucleotide synthesis procedures. The GBA gene, promoter, and a nucleic acid encoding an M2-promoting agent can then be ligated into a plasmid of interest, for example, using suitable restriction endonuclease-mediated cleavage and ligation protocols. If pluripotent cells that express non-secreted GBA and an M2-promoting agent, such as IL-25, are to be made, a bicistronic expression cassette can be used in which an IRES sequence is placed between the polynucleotide encoding GBA and the polynucleotide encoding IL-25. After the plasmid DNA is engineered, the plasmid can be used to transfect the pluripotent cells (e.g., iPS cells, ES cells, CD34+ HSCs) using, for example, electroporation or another transfection technique described herein to generate a population of pluripotent cells that express non-secreted GBA and, optionally, an M2-promoting agent. In both exemplary methods described herein, the non-secreted GBA may be expressed as a GBA fusion protein. The fusion protein may contain non-secreted GBA and a glycosylation independent lysosomal targeting (GILT) tag. Exemplary GILT tags are muteins derived from human insulin-like growth factor II (IGF-II) having an amino acid sequence that is at least 70% identical to the amino acid sequence of mature human IGF-II. These IGF-II muteins have diminished binding activity for the insulin receptor relative to the affinity of naturally-occurring human IGF-II for the insulin receptor, are resistant to furin cleavage, and bind to the human cation-independent mannose-6-phosphate receptor in a mannose-6-phosphate-independent manner. The GILT tag facilitates delivery of the secreted GBA fusion protein to the lysosome.

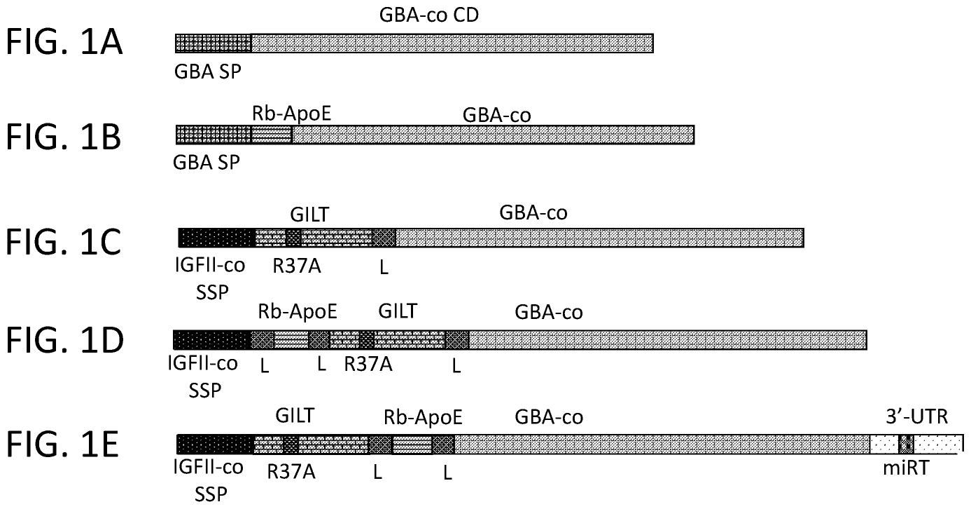

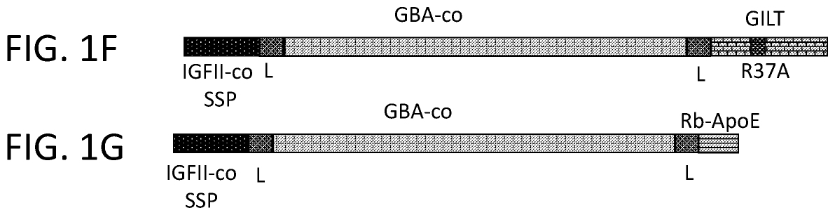

Exemplary constructs encoding GBA for use in conjunction with the compositions and methods described herein are shown in FIGS. 1A-1G.

### Example 2. Generation of a CD34+ Hematopoietic Stem Cell Expressing Secreted Glucocerebrosidase, and Optionally, an M2-Promoting Agent

An exemplary method for making pluripotent cells (e.g., CD34+ cells) that express secreted GBA and, optionally, an M2-promoting agent for use in the compositions and methods described herein is transduction. Retroviral vectors (e.g., a lentiviral vector, alpharetroviral vector, or gammaretroviral vector) containing a microglia-specific promoter, such as the CD68 promoter, a secretory signal peptide, such as the IGF-II, alpha-1 antitrypsin, or GBA secretory signal peptide, and the polynucleotide encoding GBA can be engineered using standard techniques known in the art. If pluripotent cells that express secreted GBA and an M2-promoting agent, such as IL-25, are to be made, a bicistronic expression cassette can be used in which an IRES sequence is placed between the polynucleotide encoding GBA and the polynucleotide encoding IL-25. After the retroviral vector is engineered, the retrovirus can be used to transduce pluripotent cells (e.g., iPS cells, ES cells, CD34+ HSCs) to generate a population of pluripotent cells that express non-secreted GBA and, optionally, an M2-promoting agent.

Additional exemplary methods for making pluripotent cells that express secreted GBA and, optionally, an M2-promoting agent for use in the compositions and methods described herein is transfection. Using molecular biology techniques known in the art, plasmid DNA containing a promoter, such as a microglia-specific promoter, (e.g., the CD68 promoter), a secretory signal peptide, such as the IGF-II, alpha-1 antitrypsin, or GBA secretory signal peptide, and the polynucleotide encoding GBA can be produced. For example, the GBA gene may be amplified from a human cell line using PCR-based techniques known in the art, or the gene may be synthesized, for example, using solid-phase polynucleotide synthesis procedures. The GBA gene, promoter, and a nucleic acid encoding an M2-promoting agent can then be ligated into a plasmid of interest, for example, using suitable restriction endonuclease-mediated cleavage and ligation protocols. If pluripotent cells that express secreted GBA and an M2-promoting agent, such as IL-25, are to be made, a bicistronic expression cassette can be used in which an IRES sequence is placed between the polynucleotide encoding GBA and the polynucleotide encoding IL-25. After the plasmid DNA is engineered, the plasmid can be used to transfect the pluripotent cells (e.g., iPS cells, ES cells, CD34+ HSCs) using, for example, electroporation or another transfection technique described herein to generate a population of pluripotent cells that express non-secreted GBA and, optionally, an M2-promoting agent.

In both exemplary methods described herein, the secreted GBA may be expressed as a GBA fusion protein. The fusion protein may contain secreted GBA and a glycosylation independent lysosomal targeting (GILT) tag. Exemplary GILT tags are muteins derived from human insulin-like growth factor II (IGF-II) having an amino acid sequence that is at least 70% identical to the amino acid sequence of mature human IGF-II. These IGF-II muteins have diminished binding activity for the insulin receptor relative to the affinity of naturally-occurring human IGF-II for the insulin receptor, are resistant to furin cleavage, and bind to the human cation-independent mannose-6-phosphate receptor in a mannose-6-phosphate-independent manner. The GILT tag facilitates delivery of the secreted GBA fusion protein to the lysosome. The secreted GBA fusion protein may additionally contain a peptide sequence containing the LDLRf receptor-binding (Rb) domain apolipoprotein E (ApoE) to allow for the penetrance of the GBA fusion protein across the blood-brain barrier (BBB).

### Example 3. Administration of a Population of Pluripotent Cells Expressing GBA to a Patient Suffering from Parkinson's Disease

According to the methods disclosed herein, a physician of skill in the art can treat a patient, such as a human patient, so as to reduce or alleviate symptoms of Parkinson's disease. To this end, a physician of skill in the art can administer to the human patient a population of pluripotent cells, (e.g., CD34+ cells (such as ES cells, iPS cells, HSCs, or myeloid progenitor cells)) expressing GBA. The cells may express an M2-promoting agent, such as IL-25, IL-4, IL-10, IL-13, or TGF-β. The cells can be transduced or transfected ex vivo to express GBA and, optionally, an M2-promoting agent using techniques described herein or known in the art. The population of pluripotent cells expressing GBA and, optionally, an M2-promoting agent may be administered to the patient, for example, systemically (e.g., intravenously), directly to the central nervous system (CNS) (e.g., intracerebroventricularly or stereotactically), or directly into the bone marrow (e.g., intraosseously), to treat Parkinson's disease. The cells can also be administered to the patient by multiple routes of administration, for example, intravenously and intracerebroventricularly. The cells are administered in a therapeutically effective amount, such as from 1×10⁶ cells/kg to 1×10¹² cells/kg or more (e.g., 1×10⁷ cells/kg, 1×10⁸ cells/kg, 1×10⁹ cells/kg, 1×10¹⁰ cells/kg, 1×10¹¹ cells/kg, 1×10¹² cells/kg, or more).

Before the population of pluripotent cells expressing GBA and, optionally, an M2-promoting agent is administered to the patient, one or more agents may be administered to the patient to ablate the patient's endogenous microglia and/or hematopoietic stem and progenitor cells, for example, busulfan, treosulfan, PLX3397, PLX647, PLX5622, and/or clodronate liposomes. Other methods of cell ablation well known in the art, such as irradiation, may be used alone or in combination with one or more of the aforementioned agents to ablate the patient's microglia and/or hematopoietic stem and progenitor cells. These agents and/or treatments may ablate endogenous microglia and/or hematopoietic stem and progenitor cells by at least 10% (e.g., at least 20%, 30%, 40%, 50%, 60%, 70%, 80%, 90%, 95%, or more), as assessed by PET imaging techniques known in the art. If the population of pluripotent cells expressing GBA and, optionally, an M2-promoting agent is administered to the patient after microglial ablation, the pluripotent cells can repopulate the brain, differentiating into microglia. The population of pluripotent cells expressing GBA and, optionally, an M2-promoting agent can be administered to the patient from, for example, 1 week to 1 month (e.g., 1 week, 2 weeks, 3 weeks, 4, weeks) or more after microglial ablation.

Following ablation of the patient's endogenous microglia and/or hematopoietic stem and progenitor cells, a population of CD34+ cells may be administered to the patient systemically (e.g., intravenously), or by bone marrow transplantation to reconstitute the bone marrow compartment. The number of CD34+ cells may be administered in any suitable dosage following conditioning. Non-limiting examples of dosages are about 1×10⁵ CD34+ cells/kg of recipient to about 1×10⁷ CD34+ cells/kg (e.g., from about 2×10⁵ CD34+ cells/kg to about 9×10⁶ CD34+ cells/kg, from about 3×10⁵ CD34+ cells/kg to about 8×10⁶ CD34+ cells/kg, from about 4×10⁵ CD34+ cells/kg to about 7×10⁶ CD34+ cells/kg, from about 5×10⁵ CD34+ cells/kg to about 6×10⁶ CD34+ cells/kg, from about 5×10⁵ CD34+ cells/kg to about 1×10⁷ CD34+ cells/kg, from about 6×10⁵ CD34+ cells/kg to about 1×10⁷ CD34+ cells/kg, from about 7×10⁵ CD34+ cells/kg to about 1×10⁷ CD34+ cells/kg, from about 8×10⁵ CD34+ cells/kg to about 1×10⁷ CD34+ cells/kg, from about 9×10⁵ CD34+ cells/kg to about 1×10⁷ CD34+ cells/kg, or from about 1×10⁶ CD34+ cells/kg to about 1×10⁷ CD34+ cells/kg, among others). Administration may occur prior to, or following administration of the pluripotent cells expressing GBA, and optionally one or more M2 promoting agents.

The population of pluripotent cells expressing GBA and, optionally, an M2-promoting agent can be administered to the patient in an amount sufficient to treat one or more of the pathological features of Parkinson's disease. For example, the population of pluripotent cells expressing GBA and, optionally, an M2-promoting agent can be administered in an amount sufficient to increase the quantity of M2 microglia in the brain of the patient relative to the quantity of M1 microglia in the brain of the patient. The relative increase can be measured using conventional techniques known in the art, such as by performing an ELISA on patient CSF before and after treatment to assess the level of pro-inflammatory and anti-inflammatory cytokines secreted by M1 and M2 microglia at both time points. A standard neurological examination can also be performed by the physician before and after treatment to evaluate changes in cognitive performance and motor function. The patient may be evaluated, for example, 1 month, 2 months, 3 months, 4 months, 5 months, 6 months or more following administration of the population of pluripotent cells depending on the route of administration used for treatment. A finding of reduced pro-inflammatory cytokines, increased anti-inflammatory cytokines, and/or improved cognitive or motor function following administration of a population of pluripotent cells expressing GBA and, optionally, an M2-promoting agent provides an indication that the treatment has successfully treated Parkinson's disease.

### Example 4. Disruption of Endogenous GBA in Pluripotent Cells Prior to Administration to a Patient Suffering from Parkinson's Disease

In any of the methods disclosed herein, the pluripotent stem cells, such as CD34+ cells, (e.g., those expressing a transgene encoding GBA and optionally, one or more M2-promoting agents, or non-transgene containing CD34+ cells) may be treated to disrupt the endogenous GBA prior to administration to the patient. An exemplary method of disrupting endogenous GBA in pluripotent cells is using a CRISPR/Cas system (e.g., CRISPR/Cas9) with a GBA-specific guide RNA (gRNA) to induce one or more double-strand breaks (DSB). Following non-homologous end joining (NHEJ) to repair the DSB, the presence of newly-formed indel mutations will result in endogenous GBA disruption. Alternative methods for disruption of endogenous GBA by site-specifically cleaving genomic DNA prior to the incorporation of a GBA transgene in a pluripotent stem cell include the use of zinc finger nucleases (ZFNs) and transcription activator-like effector nucleases (TALENs). Unlike the CRISPR/Cas system, these enzymes do not contain a guiding polynucleotide to localize to a specific target sequence, but instead rely on internal DNA biding domains within the enzymes to mediate target specificity. In exemplary embodiments, the pluripotent cell is manipulated ex vivo by the nuclease to decrease or reduce the expression of endogenous GBA by 10% or more (e.g., 10%, 20%, 30%, 40%, 50%, 60%, 70%, 80%, 90%, 95% or more).

### Example 5. Generation of Mammalian Cell Lines Expressing Codon-Optimized GBA

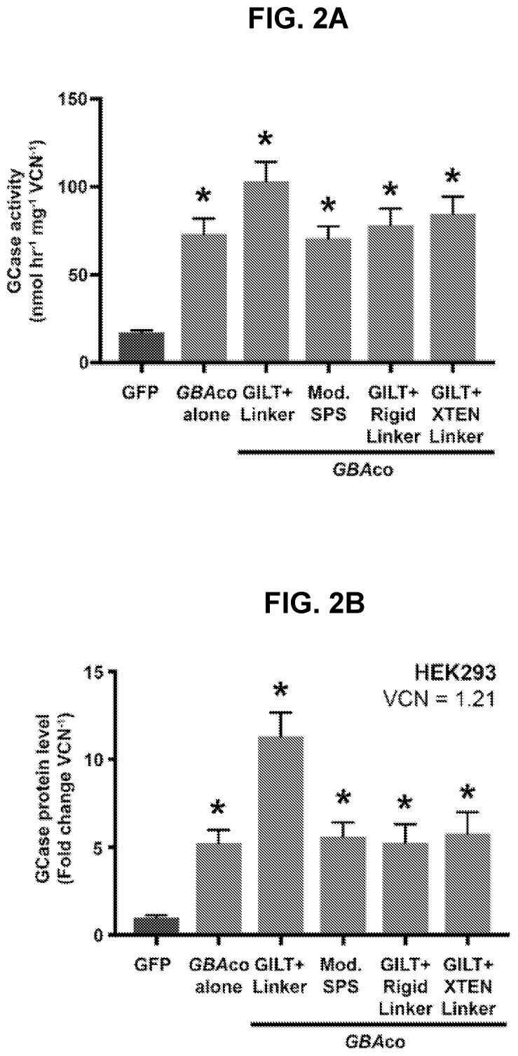

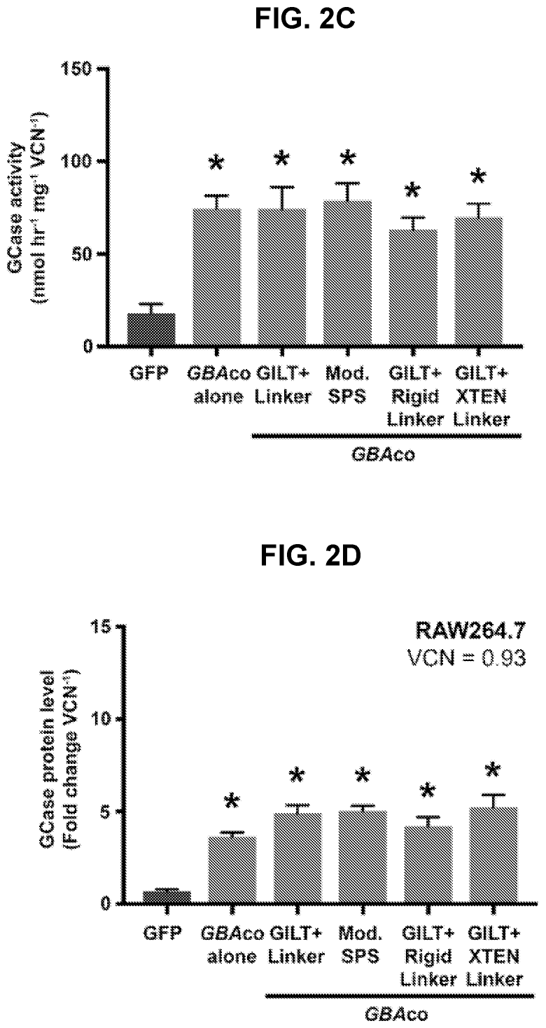

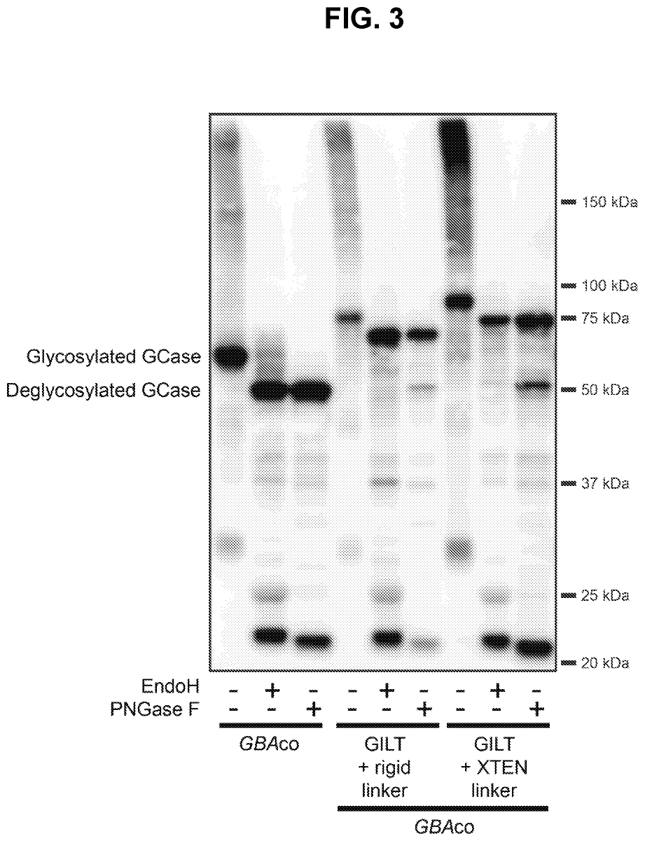

To assess the ability of lentivirally-encoded, codon-optimized GBA (GBAco) transgenes (e.g., GBAco transgenes having the codon-optimized nucleic acid sequence of any one of SEQ ID NO. 7-10) to stably express in mammalian cell lines, human HEK293T cells and mouse RAW264.7 cells were transduced in vitro. Cells were transduced with one of six engineered constructs selected from the group including: 1) green fluorescent protein (GFP); 2) GBAco alone; 3) GBAco, a glycosylation independent lysosomal targeting (GILT) tag, and a peptide linker; 4) GBAco and a modified signal peptide sequence; 5) GBAco, a GILT tag, and a rigid peptide linker; or 6) GBAco, a GILT tag, and an XTEN linker. Protein samples were harvested post-transduction to measure GBA enzymatic activity and to quantify protein levels. GBA enzymatic activity was measured in using a 4-methylumbelliferyl β-D-glucopyranoside (4MUG) substrate, which is enzymatically converted by GBA to produce a fluorescent product 4-Methylumbelliferone (4MU). Lentiviral expression of GBAco constructs in either HEK293T (vector copy number (VCN)=1.21) or RAW264.7 (VCN=0.93) cells increased GBA enzymatic activity (FIGS. 2A, 2C) and protein levels (FIGS. 2B, 2D) relative to control GFP construct (p<0.05 ANOVA with Tukey post-hoc analysis, n=3 independent transductions). Furthermore, engineered GBA proteins were detected at the predicted molecular weights, suggesting that GILT and linker peptides were stably produced (FIG. 3).

Post-translational glycosylation at the N-terminus of GBA proteins (e.g., N-linked glycosylation) is critical for GBA function. Thus, engineered GBA proteins were tested for N-linked glycosylation in HEK293T cells in vitro. We assayed the electrophoretic mobility shift of the engineered GBA proteins after enzymatic digestion with either EndoH or PNGase F glycosidases that remove N-linked sugars from glycoproteins. Western blot analysis of cell lysates revealed increased electrophoretic mobility following de-glycosylation in all of the tested constructs (e.g., GBAco; GBAco, a GILT tag, and a rigid linker; or GBAco, a GILT tag, and an XTEN linker; FIG. 3), indicating that engineered GBA proteins are glycosylated in mammalian cell lines.

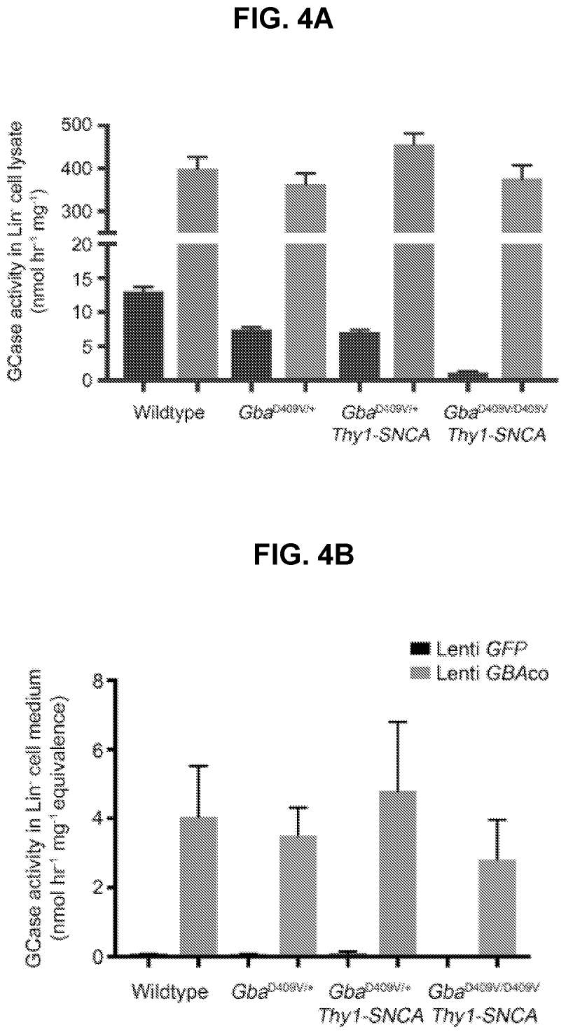

To determine whether engineered GBA constructs can be stably expressed in hematopoietic stem cells, lentivirally-encoded constructs were transduced into mouse lineage negative (Lin⁻) hematopoietic stem cells isolated from the bone marrow of mouse models of GBA deficiency (Gba^{D409V/+} n=3; Thy1-SNCA; Gba^{D409V/+} n=1; Thy1-SNCA; Gba^{D409V/D409V} n=2; wildtype (WT) n=2). Five days after transduction with either lentiviral constructs encoding GFP or GBAco (multiplicity of infection=80), Lin⁻ cells were >83% viable (assayed by trypan blue dye exclusion) and remained multipotent (>50 colony forming units). Enzymatic assays of Lin⁻ cells demonstrated that the heterozygous and homozygous Gba mutations reduced GBA activity by 43% and 92%, respectively, in the absence of GBA transgenes (WT: 13.04±0.644 nmol hr⁻¹; Gba^{D409V/+}; 7.49±0.293 nmol hr⁻¹ mg⁻¹; Thy1-SNCA; Gba^{D409V/+}: 7.14±0.252 nmol hr⁻¹ mg⁻¹; Thy1-SNCA; Gba^{D409V/D409V}:1.20±0.114 nmol hr⁻¹ mg⁻¹; p<0.001, ANOVA with Tukey post-hoc analysis, FIG. 4A). Importantly, GBAco transduction of Lin⁻ cells significantly increased GBA enzymatic activity to similar levels across all tested GBAco constructs relative to GFP control (p<0.001, ANOVA with Tukey post-hoc analysis; FIG. 4A). Furthermore, GBAco expression led to the detection of GBA activity in conditioned media from the Lin⁻ cells by Western blot (FIG. 4B). Combined, these findings demonstrate that lentiviral GBAco constructs produce a functional GBA enzyme in hematopoietic stem cells (e.g., mouse Lin⁻ cells) and can rescue GBA activity and expression levels in mouse models of GBA deficiency.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A method of treating Parkinson's disease in a subject, the method comprising administering to the subject a composition comprising a population of pluripotent cells that express a transgene encoding glucocerebrosidase (GBA).

2. The method of claim 1, wherein the GBA is full-length GBA.

3. The method of claim 1, wherein the GBA is a catalytic domain of GBA.

4. The method of any one of claims 1-3, wherein the GBA has an amino acid sequence that is at least 85% identical to the amino acid sequence of SEQ ID NO. 1.

5. The method of claim 4, wherein the GBA has an amino acid sequence that is at least 90% identical to the amino acid sequence of SEQ ID NO. 1.

6. The method of claim 5, wherein the GBA has an amino acid sequence that is at least 95% identical to the amino acid sequence of SEQ ID NO. 1.

7. The method of claim 6, wherein the GBA has an amino acid sequence that is at least 95% identical to the amino acid sequence of SEQ ID NO. 1.

8. The method of any one of claims 1-3, wherein the GBA has an amino acid sequence that is at least 85% identical to the amino acid sequence of SEQ ID NO. 5.

9. The method of claim 8, wherein the GBA has an amino acid sequence that is at least 90% identical to the amino acid sequence of SEQ ID NO. 5.

10. The method of claim 9, wherein the GBA has an amino acid sequence that is at least 95% identical to the amino acid sequence of SEQ ID NO. 5.

11. The method of claim 10, wherein the GBA has the amino acid sequence of SEQ ID NO. 5.

12. The method of any one of claims 1-3, wherein the transgene encoding GBA has a nucleic acid sequence that is at least 85% identical to the nucleic acid sequence of SEQ ID NO. 6.

13. The method of claim 12, wherein the transgene encoding GBA has a nucleic acid sequence that is at least 90% identical to the nucleic acid sequence of SEQ ID NO. 6.

14. The method of claim 13, wherein the transgene encoding GBA has a nucleic acid sequence that is at least 95% identical to the nucleic acid sequence of SEQ ID NO. 6.

15. The method of claim 14, wherein the transgene encoding GBA has the nucleic acid sequence of SEQ ID NO. 6.

16. The method of any one of claims 1-3, wherein the transgene encoding GBA has a nucleic acid sequence that is at least 85% identical to the nucleic acid sequence of SEQ ID NO. 7.

17. The method of claim 16, wherein the transgene encoding GBA has a nucleic acid sequence that is at least 90% identical to the nucleic acid sequence of SEQ ID NO. 7.

18. The method of claim 17, wherein the transgene encoding GBA has a nucleic acid sequence that is at least 95% identical to the nucleic acid sequence of SEQ ID NO. 7.

19. The method of claim 18, wherein the transgene encoding GBA has the nucleic acid sequence of SEQ ID NO. 7.

20. The method of any one of claims 1-3, wherein the transgene encoding GBA has a nucleic acid sequence that is at least 85% identical to the nucleic acid sequence of SEQ ID NO. 11.

21. The method of claim 20, wherein the transgene encoding GBA has a nucleic acid sequence that is at least 90% identical to the nucleic acid sequence of SEQ ID NO. 11.

22. The method of claim 21, wherein the transgene encoding GBA has a nucleic acid sequence that is at least 95% identical to the nucleic acid sequence of SEQ ID NO. 11.

23. The method of claim 22, wherein the transgene encoding GBA has the nucleic acid sequence of SEQ ID NO. 11.

24. The method of any one of claims 1-23, wherein the GBA comprises a signal peptide.

25. The method of claim 24, wherein the signal peptide has an amino acid sequence that is at least 85% identical to the amino acid sequence of SEQ ID NO. 16.

26. The method of claim 25, wherein the signal peptide has an amino acid sequence that is at least 90% identical to the amino acid sequence of SEQ ID NO. 16.

27. The method of claim 26, wherein the signal peptide has an amino acid sequence that is at least 95% identical to the amino acid sequence of SEQ ID NO. 16.

28. The method of claim 27, wherein the signal peptide has the amino acid sequence of SEQ ID NO. 16.

29. The method of any one of claims 24-28, wherein the signal peptide is encoded by a polynucleotide having a nucleic acid sequence that is at least 85% identical to the nucleic acid sequence of SEQ ID NO. 20.

30. The method of claim 29, wherein the signal peptide is encoded by a polynucleotide having a nucleic acid sequence that is at least 90% identical to the nucleic acid sequence of SEQ ID NO. 20.

31. The method of claim 30, wherein the signal peptide is encoded by a polynucleotide having a nucleic acid sequence that is at least 95% identical to the nucleic acid sequence of SEQ ID NO. 20.

32. The method of claim 31, wherein the signal peptide is encoded by a polynucleotide having the nucleic acid sequence of SEQ ID NO. 20.

33. The method of any one of claims 1-19, wherein the transgene encoding GBA encodes non-secreted GBA.

34. The method of claim 33, wherein the transgene encoding non-secreted GBA comprises a signal peptide.

35. The method of claim 34, wherein the signal peptide is a GBA signal peptide.

36. The method of any one of claims 1-19, wherein the transgene encoding GBA encodes secreted GBA.

37. The method of claim 36, wherein the transgene encoding secreted GBA comprises a secretory signal peptide.

38. The method of claim 37, wherein the secretory signal peptide is an alpha-1 antitrypsin secretory signal peptide.

39. The method of claim 38, wherein the secretory signal peptide is an insulin-like growth factor II (IGF-II) secretory signal peptide.

40. The method of any one of claims 1-3, wherein the transgene encoding GBA encodes a GBA fusion protein.

41. The method of claim 40, wherein the GBA fusion protein has an amino acid sequence that is at least 85% identical to the amino acid sequence of SEQ ID NO. 2.

42. The method of claim 41, wherein the GBA fusion protein has an amino acid sequence that is at least 90% identical to the amino acid sequence of SEQ ID NO. 2.

43. The method of claim 42, wherein the GBA fusion protein has an amino acid sequence that is at least 95% identical to the amino acid sequence of SEQ ID NO. 2.

44. The method of claim 43, wherein the GBA fusion protein has the amino acid sequence of SEQ ID NO. 2.

45. The method of claim 40, wherein the GBA fusion protein has an amino acid sequence that is at least 85% identical to the amino acid sequence of SEQ ID NO. 3.

46. The method of claim 45, wherein the GBA fusion protein has an amino acid sequence that is at least 90% identical to the amino acid sequence of SEQ ID NO. 3.

47. The method of claim 46, wherein the GBA fusion protein has an amino acid sequence that is at least 95% identical to the amino acid sequence of SEQ ID NO. 3.

48. The method of claim 47, wherein the GBA fusion protein has the amino acid sequence of SEQ ID NO. 3.

49. The method of claim 40, wherein the GBA fusion protein has an amino acid sequence that is at least 85% identical to the amino acid sequence of SEQ ID NO. 4.

50. The method of claim 49, wherein the GBA fusion protein has an amino acid sequence that is at least 90% identical to the amino acid sequence of SEQ ID NO. 4.

51. The method of claim 50, wherein the GBA fusion protein has an amino acid sequence that is at least 95% identical to the amino acid sequence of SEQ ID NO. 4.

52. The method of claim 51, wherein the GBA fusion protein has the amino acid sequence of SEQ ID NO. 4.

53. The method of claim 40, wherein the transgene encoding the GBA fusion protein has a nucleic acid sequence that is at least 85% identical to the nucleic acid sequence of SEQ ID NO. 8.

54. The method of claim 53, wherein the transgene encoding the GBA fusion protein has a nucleic acid sequence that is at least 90% identical to the nucleic acid sequence of SEQ ID NO. 8.

55. The method of claim 54, wherein the transgene encoding the GBA fusion protein has a nucleic acid sequence that is at least 95% identical to the nucleic acid sequence of SEQ ID NO. 8

56. The method of claim 55, wherein the transgene encoding the GBA fusion protein has a nucleic acid sequence of SEQ ID NO. 8.

57. The method of claim 40, wherein the transgene encoding the GBA fusion protein has a nucleic acid sequence that is at least 85% identical to the nucleic acid sequence of SEQ ID NO. 9.

58. The method of claim 57, wherein the transgene encoding the GBA fusion protein has a nucleic acid sequence that is at least 90% identical to the nucleic acid sequence of SEQ ID NO. 9.

59. The method of claim 58, wherein the transgene encoding the GBA fusion protein has a nucleic acid sequence that is at least 95% identical to the nucleic acid sequence of SEQ ID NO. 9.

60. The method of claim 59, wherein the transgene encoding the GBA fusion protein has a nucleic acid sequence of SEQ ID NO. 9.

61. The method of claim 40, wherein the transgene encoding the GBA fusion protein has a nucleic acid sequence that is at least 85% identical to the nucleic acid sequence of SEQ ID NO. 10.

62. The method of claim 61, wherein the transgene encoding the GBA fusion protein has a nucleic acid sequence that is at least 90% identical to the nucleic acid sequence of SEQ ID NO. 10.

63. The method of claim 62, wherein the transgene encoding the GBA fusion protein has a nucleic acid sequence that is at least 95% identical to the nucleic acid sequence of SEQ ID NO. 10.

64. The method of claim 63, wherein the transgene encoding the GBA fusion protein has a nucleic acid sequence of SEQ ID NO. 10.

65. The method of claim any one of claims 40-64, wherein the GBA fusion protein comprises GBA and a glycosylation independent lysosomal targeting (GILT) tag.

66. The method of claim 65, wherein the GILT tag comprises a human IGF-II mutein having an amino acid sequence that is at least 70% identical to the amino acid sequence of mature human IGF-II (SEQ ID NO. 12), and having diminished binding affinity for the insulin receptor relative to the affinity of naturally-occurring human IGF-II for the insulin receptor, wherein the IGF-II mutein is resistant to furin cleavage and binds to the human cation-independent mannose-6-phosphate receptor in a mannose-6-phosphate-independent manner.

67. The method of claim 66, wherein the IGF-II mutein comprises a mutation within a region corresponding to amino acids 30-40 of SEQ ID NO. 12, and wherein the mutation abolishes at least one furin protease cleavage site.

68. The method of claim 67, wherein the mutation is an amino acid substitution, deletion, and/or insertion.

69. The method of claim 68, wherein the mutation is a Lys or Ala amino acid substitution at a position corresponding to Arg37 or Arg40 of SEQ ID NO. 12.

70. The method of claim 69, wherein the mutation is a deletion or replacement of amino acid residues corresponding to positions selected form the group consisting of 31-40, 32-40, 33-40, 34-40, 30-39, 31-39, 32-39, 34-37, 33-39, 35-39, 36-39, 37-40, 34-40 of SEQ ID NO. 12, and combinations thereof.

71. The method of any one of claims 65-70, wherein the GILT tag has an amino acid sequence that is at least 70% identical to the amino acid sequence of SEQ ID NO. 13.

72. The method of claim 71, wherein the GILT tag has an amino acid sequence that is at least 80% identical to the amino acid sequence of SEQ ID NO. 13.

73. The method of claim 72, wherein the GILT tag has an amino acid sequence that is at least 90% identical to the amino acid sequence of SEQ ID NO. 13.

74. The method of claim 73, wherein the GILT tag has the amino acid sequence of SEQ ID NO. 13.

75. The method of any one of claims 65-70, wherein the GILT tag has an amino acid sequence that is at least 70% identical to the amino acid sequence of SEQ ID NO. 14.

76. The method of claim 75, wherein the GILT tag has an amino acid sequence that is at least 80% identical to the amino acid sequence of SEQ ID NO. 14.

77. The method of claim 76, wherein the GILT tag has an amino acid sequence that is at least 90% identical to the amino acid sequence of SEQ ID NO. 14.

78. The method of claim 77, wherein the GILT tag has the amino acid sequence of SEQ ID NO. 14.

79. The method of any one of claims 65-70, wherein the GILT tag has an amino acid sequence that is at least 70% identical to the amino acid sequence of SEQ ID NO. 15.

80. The method of claim 79, wherein the GILT tag has an amino acid sequence that is at least 80% identical to the amino acid sequence of SEQ ID NO. 15.

81. The method of claim 80, wherein the GILT tag has an amino acid sequence that is at least 90% identical to the amino acid sequence of SEQ ID NO. 15.

82. The method of claim 81, wherein the GILT tag has the amino acid sequence of SEQ ID NO. 15.

83. The method of any one of claims 65-82, wherein the GILT tag is encoded by a polynucleotide having a nucleic acid sequence that is at least 85% identical to the nucleic acid sequence of SEQ ID NO. 17.

84. The method of claim 83, wherein the GILT tag is encoded by a polynucleotide having a nucleic acid sequence that is at least 90% identical to the nucleic acid sequence of SEQ ID NO. 17.

85. The method of claim 84, wherein the GILT tag is encoded by a polynucleotide having a nucleic acid sequence that is at least 95% identical to the nucleic acid sequence of SEQ ID NO. 17.

86. The method of claim 85, wherein the GILT tag is encoded by a polynucleotide having the nucleic acid sequence of SEQ ID NO. 17.

87. The method of any one of claims 65-82, wherein the GILT tag is encoded by a polynucleotide having a nucleic acid sequence that is at least 85% identical to the nucleic acid sequence of SEQ ID NO. 18.

88. The method of claim 87, wherein the GILT tag is encoded by a polynucleotide having a nucleic acid sequence that is at least 90% identical to the nucleic acid sequence of SEQ ID NO. 18.

89. The method of claim 88, wherein the GILT tag is encoded by a polynucleotide having a nucleic acid sequence that is at least 95% identical to the nucleic acid sequence of SEQ ID NO. 18.

90. The method of claim 89, wherein the GILT tag is encoded by a polynucleotide having the nucleic acid sequence of SEQ ID NO. 18.

91. The method of any one of claims 65-82, wherein the GILT tag is encoded by a polynucleotide having a nucleic acid sequence that is at least 85% identical to the nucleic acid sequence of SEQ ID NO. 19.

92. The method of claim 91, wherein the GILT tag is encoded by a polynucleotide having a nucleic acid sequence that is at least 90% identical to the nucleic acid sequence of SEQ ID NO. 19.

93. The method of claim 92, wherein the GILT tag is encoded by a polynucleotide having a nucleic acid sequence that is at least 95% identical to the nucleic acid sequence of SEQ ID NO. 19.

94. The method of claim 93, wherein the GILT tag is encoded by a polynucleotide having the nucleic acid sequence of SEQ ID NO. 19.

95. The method of any one of claims 40-94, wherein the GBA fusion protein comprises a receptor-binding (Rb) domain of apolipoprotein E (ApoE).

96. The method of claim 95, wherein the Rb domain comprises a portion of ApoE having the amino acid sequence of residues 25-185, 50-180, 75-175, 100-170, 125-160, or 130-150 of SEQ ID NO. 21.

97. The method of claim 96, wherein the Rb domain comprises a region having at least 70% sequence identity to the amino acid sequence of residues 159-167 of SEQ ID NO. 21.

98. The method of any one of claims 1-97, wherein the transgene encoding GBA further comprises a micro RNA (miRNA) targeting sequence in the 3′-UTR.

99. The method of claim 98, wherein the miRNA targeting sequence is a miR-126 targeting sequence.

100. The method of any one of claims 36-99, wherein the secreted GBA penetrates the blood brain barrier (BBB) in the subject.

101. The method of any one of claims 1-100, wherein the Parkinson's disease is GBA-associated Parkinson's disease.

102. The method of any one of claims 1-101, wherein the pluripotent cells are CD34+ cells.

103. The method of claim 102, wherein the CD34+ cells are embryonic stem cells.

104. The method of claim 102, wherein the CD34+ cells are induced pluripotent stem cells.

105. The method of claim 102, wherein the CD34+ cells are hematopoietic stem cells.

106. The method of claim 102, wherein the CD34+ cells are myeloid progenitor cells.

107. The method of any one of claims 1-106, wherein a population of endogenous microglia in the subject has been ablated prior to administration of the composition.

108. The method of any one of claims 1-106, the method comprising ablating a population of endogenous microglia in the subject prior to administering the composition to the subject.

109. The method of claim 107 or 108, wherein the microglia are ablated using an agent selected from the group consisting of busulfan, PLX3397, PLX647, PLX5622, treosulfan, and clodronate liposomes, by radiation therapy, or a combination thereof.

110. The method of any one of claims 1-109, wherein the composition is administered systemically to the subject.

111. The method of claim 110, wherein the composition is administered to the subject by way of intravenous injection.

112. The method of any one of claims 1-109, wherein the composition is administered directly to the central nervous system of the subject.

113. The method of claim 112, wherein the composition is administered to the subject by way of intracerebroventricular injection, stereotactic injection, or a combination thereof.

114. The method of any one of claims 1-109, wherein the composition is administered directly to the bone marrow of the subject.

115. The method of claim 114, wherein the composition is administered to the subject by way of intraosseous injection.

116. The method of any one of claims 1-109, wherein the composition is administered to the subject by way of a bone marrow transplant comprising the composition.

117. The method of any one of claims 1-109, wherein the composition is administered to the subject by way of intracerebroventricular injection.

118. The method of any one of claims 1-109, wherein the composition is administered to the subject by way of intravenous injection.

119. The method of any one of claims 1-109, wherein the composition is administered to the subject by direct administration to the central nervous system of the subject and by systemic administration.

120. The method of claim 119, wherein the composition is administered to the subject by way of intracerebroventricular injection and intravenous injection.

121. The method of any one of claims 1-120, the method further comprising administering to the subject a population of CD34+ cells.

122. The method of claim 121, wherein the population of CD34+ cells is administered to the subject prior to administration of the composition.

123. The method of claim 121, wherein the population of CD34+ cells is administered to the subject following administration of the composition.

124. The method of any one of claims 121-123, wherein the CD34+ cells are selected from the group consisting of embryonic stem cells, induced pluripotent stem cells, hematopoietic stem cells, and myeloid progenitor cells.

125. The method of any one of claims 121-124, wherein the CD34+ cells are not modified to express a transgene encoding GBA.

126. The method of any one of claims 121-125, wherein the CD34+ cells are administered to the subject systemically.

127. The method of claim 126, wherein the CD34+ cells are administered to the subject by way of intravenous injection.

128. The method of any one of claims 1-127, wherein, prior to administration of the composition to the subject, endogenous GBA is disrupted in the pluripotent cells.

129. The method of any one of claims 1-128, wherein, prior to administration of the composition to the subject, endogenous GBA is disrupted in the subject.

130. The method of claim 129, wherein, prior to the administration of the composition to the subject, endogenous GBA is disrupted in a population of neurons in the subject.

131. The method of claim 128, wherein the endogenous GBA is disrupted by contacting the pluripotent cells with a nuclease that catalyzes cleavage of an endogenous GBA nucleic acid in the pluripotent cells.

132. The method of claim 131, wherein the nuclease is a clustered regularly interspaced short palindromic repeats (CRISPR)-associated protein.

133. The method of claim 132, wherein the CRISPR-associated protein is CRISPR associated protein 9 (Cas9).

134. The method of claim 131, wherein the nuclease is a transcription activator-like effector nuclease, a meganuclease, or a zinc finger nuclease.

135. The method of any one of claims 128-130, wherein the endogenous GBA is disrupted by administering an inhibitory RNA molecule to the pluripotent cells with, the subject, or the population of neurons.

136. The method of claim 135, wherein the inhibitory RNA molecule is a short interfering RNA, a short hairpin RNA, or a miRNA.

137. The method of any one of claims 1-136, wherein the pluripotent cells further express one or more transgenes that each encode an M2-promoting agent.

138. The method of any one of claims 1-136, wherein the pluripotent cells express two transgenes that each encode an M2-promoting agent.

139. The method of claim 137 or 138, wherein at least one of the one or more transgenes that each encode an M2-promoting agent encodes a cytokine selected from the group consisting of interleukin-25 (IL-25), interleukin-4 (IL-4), interleukin-10 (IL-10), interleukin-13 (IL-13), and transforming growth factor beta (TGF-β).

140. The method of claim 139, wherein at least one of the one or more transgenes that each encode an M2-promoting agent encodes IL-25.

141. The method of claim 137 or 138, wherein at least one of the one or more transgenes that each encode an M2-promoting agent encodes an agent selected from the group consisting of a glucocorticoid receptor, a peroxisome proliferator-activated receptor (PPAR), PPARγ, PPARβ/δ, an estrogen receptor, nuclear receptor subfamily 4 group A member 2 (NR4A2), lysine demethylase 6B (KDM6B), MSH homeobox 3 (MSX3), family with sequence similarity 19 (chemokine (C—C motif)-like), member A3 (FAM19A3), nuclear factor NF-Kappa-B P50 subunit (NF-κB p50), microRNA 124 (miR124), microRNA 21 (miR21), and microRNA 181c (miR181c), C-X3-C motif chemokine ligand 1 (CX3CL1), C-X3-C motif chemokine receptor 1 (CX3CR1), CD200 molecule (CD200), CD200 receptor 1 (CD200R), complement factor H (CFH), leukocyte surface antigen CD47 (CD47), complement decay-accelerating factor (CD55), trophoblast leukocyte common antigen (CD46), adhesion G protein-coupled receptor E5 (ADGRE5), signal regulatory protein alpha (SIRPA), and siglecs.

142. The method of any one of claims 1-141, wherein the pluripotent cells are autologous cells.

143. The method of any one of claims 1-141, wherein the pluripotent cells are allogeneic cells.

144. The method of any one of claims 1-141, wherein the pluripotent cells are transduced ex vivo to express the GBA.

145. The method of any one of claims 137-144, wherein the pluripotent cells are transduced ex vivo to express the GBA and the one or more M2-promoting agents.

146. The method of claim 144 or 145, wherein the pluripotent cells are transduced with a viral vector selected from the group consisting of an adeno-associated virus (AAV), an adenovirus, a parvovirus, a coronavirus, a rhabdovirus, a paramyxovirus, a picornavirus, an alphavirus, a herpes virus, a poxvirus, and a Retroviridae family virus.

147. The method of claim 146, wherein the viral vector is a Retroviridae family viral vector.

148. The method of claim 147, wherein the Retroviridae family viral vector is a lentiviral vector.

149. The method of claim 147, wherein the Retroviridae family viral vector is an alpharetroviral vector.

150. The method of claim 147, wherein the Retroviridae family viral vector is a gammaretroviral vector.

151. The method of any one of claims 147-150, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

152. The method of claim 146, wherein the viral vector is an AAV selected from the group consisting of AAV1, AAV2, AAV3, AAV4, AAV5, A AV6, AAV7, AAV8, AAV9, AAV10, and AAVrh74.

153. The method of claim 146, wherein the viral vector is a pseudotyped viral vector.

154. The method of claim 153, wherein the pseudotyped viral vector selected from the group consisting of a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

155. The method of any one of claims 144-154, wherein the pluripotent cells are transduced to express the GBA and the one or more M2-promoting agents from separate, monocistronic expression cassettes.

156. The method of any one of claims 144-154, wherein the pluripotent cells are transduced to express the GBA and the one or more M2-promoting agents from a polycistronic expression cassette.

157. The method of claim 156, wherein the pluripotent cells express a single M2-promoting agent, and wherein the pluripotent cells are transduced to express the GBA and the M2-promoting agent from a bicistronic expression cassette.

158. The method of claim 156, wherein the polycistronic expression cassette comprises an internal ribosomal entry site (IRES) positioned between a polynucleotide encoding the GBA and a polynucleotide encoding one of the one or more M2-promoting agents.

159. The method of claim 156, wherein the polycistronic expression cassette comprises a foot-and-mouth disease virus 2A (FMDV 2A) polynucleotide positioned between a polynucleotide encoding the GBA and a polynucleotide encoding one of the one or more M2-promoting agents.

160. The method of claim 156, wherein the pluripotent cells express GBA, a first M2-promoting agent, and a second M2-promoting agent from a single polycistronic expression cassette, and wherein the polycistronic expression cassette comprises a first FMDV 2A polynucleotide positioned between a polynucleotide encoding the GBA and a polynucleotide encoding the first M2-promoting agent, and wherein the polycistronic expression cassette further comprises a second FMDV 2A polynucleotide positioned between the polynucleotide encoding the first M2-promoting agent and a polynucleotide encoding the second M2-promoting agent.

161. The method of any one of claims 1-143, wherein the pluripotent cells are transfected ex vivo to express the GBA.

162. The method of any one of claims 137-144, wherein the pluripotent cells are transfected ex vivo to express the GBA and the one or more M2-promoting agents.

163. The method of claim 161 or 162, wherein the pluripotent cells are transfected using:
a) an agent selected from the group consisting of a cationic polymer, diethylaminoethyl-dextran, polyethylenimine, a cationic lipid, a liposome, calcium phosphate, an activated dendrimer, and a magnetic bead; or
b) a technique selected from the group consisting of electroporation, Nucleofection, squeeze-poration, sonoporation, optical transfection, Magnetofection, and impalefection.

164. The method of claim 162 or 163, wherein the pluripotent cells are transfected ex vivo to express the GBA and the one or more M2-promoting agents from separate, monocistronic expression cassettes.

165. The method of claim 164, wherein the monocistronic expression cassettes are located within two or more separate plasmids.

166. The method of claim 164, wherein the monocistronic expression cassettes are located on a single plasmid.

167. The method of claim 162 or 163, wherein the pluripotent cells are transfected ex vivo to express the GBA and the one or more M2-promoting agents from a polycistronic expression cassette.

168. The method of claim 167, wherein the pluripotent cells express a single M2-promoting agent, and wherein the pluripotent cells are transfected ex vivo to express the GBA and the M2-promoting agent from a bicistronic expression cassette.

169. The method of claim 167, wherein the polycistronic expression cassette comprises an IRES positioned between a polynucleotide encoding the GBA and a polynucleotide encoding one of the one or more M2-promoting agents.

170. The method of claim 167, wherein the polycistronic expression cassette comprises an FMDV 2A polynucleotide positioned between a polynucleotide encoding the GBA and a polynucleotide encoding one of the one or more M2-promoting agents.

171. The method of claim 167, wherein the pluripotent cells express GBA, a first M2-promoting agent, and a second M2-promoting agent from a single polycistronic expression cassette, and wherein the polycistronic expression cassette comprises a first FMDV 2A polynucleotide positioned between a polynucleotide encoding the GBA and a polynucleotide encoding the first M2-promoting agent, and wherein the polycistronic expression cassette further comprises a second FMDV 2A polynucleotide positioned between the polynucleotide encoding the first M2-promoting agent and a polynucleotide encoding the second M2-promoting agent.

172. The method of any one of claims 137-171, wherein expression of the GBA and/or the one or more M2-promoting agents in the pluripotent cells is driven using a ubiquitous promoter.

173. The method of claim 172, wherein the ubiquitous promoter is selected from the group consisting of elongation factor 1-alpha and phosphoglycerate kinase 1.

174. The method of any one of claims 137-171, wherein expression of the GBA and/or the one or more M2-promoting agents in the pluripotent cells is driven using a tissue-specific promoter.

175. The method of claim 174, wherein the tissue-specific promoter is selected from the group consisting of CD68 molecule, C-X3-C motif chemokine receptor 1, integrin subunit alpha M, allograft inflammatory factor 1, purinergic receptor P2Y12, transmembrane protein 119, and colony stimulating factor 1 receptor.

176. The method of any one of claims 1-175, wherein the transgene encoding GBA is operably linked to a nucleic acid encoding a protein destabilizing domain.

177. The method of claim 176, wherein the destabilizing domain is an FK506 binding protein 1A (FKBP12) destabilizing domain.

178. The method of claim 177, wherein the FPBP12 destabilizing domain is an FKBP12 mutant selected from the group consisting of F15S, V24A, H25R, E60G, L106P, M66T, R71G, D100G, D100N, E102G, and K105I.

179. The method of claim 177 or 178, wherein the method further comprises administering Shield-1 to the subject in a quantity sufficient to induce expression of functional GBA.

180. The method of claim 176, wherein the destabilizing domain is an E. coli dihydrofolate reductase (ecDHFR) destabilizing domain.

181. The method of claim 180, wherein the method further comprises administering trimethoprim to the subject in a quantity sufficient to induce expression of functional GBA.

182. The method of claim 176, wherein the destabilizing domain is a human estrogen receptor ligand binding domain (ERLBD) destabilizing domain.

183. The method of claim 182, wherein the method further comprises administering CMP8 or 4-hydroxytamoxifen to the subject in a quantity sufficient to induce expression of functional GBA.

184. The method of any one of claims 137-183, wherein one or more polynucleotides encoding each of the one or more M2-promoting agents in the pluripotent cells is operably linked to a protein destabilizing domain.

185. The method of claim 184, wherein the destabilizing domain is an FKBP12 destabilizing domain.

186. The method of claim 185, wherein the FPBP12 destabilizing domain is an FKBP12 mutant selected from the group consisting of F15S, V24A, H25R, E60G, L106P, M66T, R71G, D100G, D100N, E102G, and K105I.

187. The method of claim 185 or 186, wherein the method further comprises administering Shield-1 to the subject in a quantity sufficient to induce expression of a functional M2-promoting agent.

188. The method of claim 184, wherein the destabilizing domain is an ecDHFR destabilizing domain.

189. The method of claim 188, wherein the method further comprises administering trimethoprim to the subject in a quantity sufficient to induce expression of a functional M2-promoting agent.

190. The method of claim 184, wherein the destabilizing domain is a human ERLBD destabilizing domain.

191. The method of claim 190, wherein the method further comprises administering CMP8 or 4-hydroxytamoxifen to the subject in a quantity sufficient to induce expression of a functional M2-promoting agent.

192. The method of any one of claims 1-191, wherein the composition is administered to the subject in an amount sufficient to:
a) increase the quantity of M2 microglia in the brain of the subject relative to the quantity of M1 microglia in the brain of the subject;
b) decrease the level of one or more proinflammatory cytokines in the brain of the subject;
c) increase the level of one or more anti-inflammatory cytokines in the brain of the subject;
d) improve the cognitive performance of the subject;
e) improve the motor function of the subject;
f) reduce dopaminergic neuron loss in the subject; and/or
g) reduce α-synuclein levels or aggregation thereof in the subject.

193. The method of any one of claims 1-192, wherein the subject is a human.

194. A composition comprising a population of pluripotent cells that express (i) a first transgene encoding non-secreted GBA; and (ii) one or more transgenes that each encode an M2-promoting agent.

195. A composition comprising a population of pluripotent cells that express (i) a first transgene encoding secreted GBA; and (ii) one or more transgenes that each encode an M2-promoting agent.

196. The composition of claim 194 or 195, wherein the GBA is full-length GBA.

197. The composition of claim 194 or 195, wherein the GBA is a catalytic domain of GBA.

198. The composition of claim any one of claims 194-197, wherein the GBA has an amino acid sequence that is at least 85% identical to the amino acid sequence of SEQ ID NO. 1.

199. The composition of claim 198, wherein the GBA has an amino acid sequence that is at least 90% identical to the amino acid sequence of SEQ ID NO. 1.

200. The composition of claim 199, wherein the GBA has an amino acid sequence that is at least 95% identical to the amino acid sequence of SEQ ID NO. 1.

201. The composition of claim 200, wherein the GBA has an amino acid sequence that is at least 95% identical to the amino acid sequence of SEQ ID NO. 1.

202. The composition of claim any one of claims 195-197, wherein the GBA has an amino acid sequence that is at least 85% identical to the amino acid sequence of SEQ ID NO. 5.

203. The composition of claim 202, wherein the GBA has an amino acid sequence that is at least 90% identical to the amino acid sequence of SEQ ID NO. 5.

204. The composition of claim 203, wherein the GBA has an amino acid sequence that is at least 95% identical to the amino acid sequence of SEQ ID NO. 5.

205. The composition of claim 204, wherein the GBA has the amino acid sequence of SEQ ID NO. 5.

206. The composition of claim any one of claims 194-197, wherein the transgene encodes a polynucleotide having at least 85% sequence identity to the nucleic acid sequence of SEQ ID NO. 6.

207. The composition of claim 206, wherein the transgene encodes a polynucleotide having at least 90% sequence identity to the nucleic acid sequence of SEQ ID NO. 6.

208. The composition of claim 207, wherein the transgene encodes a polynucleotide having at least 95% sequence identity to the nucleic acid sequence of SEQ ID NO. 6.

209. The composition of claim 208, wherein the transgene encodes a polynucleotide having the nucleic acid sequence of SEQ ID NO. 6.

210. The composition of claim any one of claims 194-197, wherein the transgene encodes a polynucleotide having at least 85% sequence identity to the nucleic acid sequence of SEQ ID NO. 7.

211. The composition of claim 210, wherein the transgene encodes a polynucleotide having at least 90% sequence identity to the nucleic acid sequence of SEQ ID NO. 7.

212. The composition of claim 211, wherein the transgene encodes a polynucleotide having at least 95% sequence identity to the nucleic acid sequence of SEQ ID NO. 7.

213. The composition of claim 212, wherein the transgene encodes a polynucleotide having the nucleic acid sequence of SEQ ID NO. 7.

214. The composition of claim any one of claims 195-197, wherein the transgene encodes a polynucleotide having at least 85% sequence identity to the nucleic acid sequence of SEQ ID NO. 11.

215. The composition of claim 214, wherein the transgene encodes a polynucleotide having at least 90% sequence identity to the nucleic acid sequence of SEQ ID NO. 11.

216. The composition of claim 215, wherein the transgene encodes a polynucleotide having at least 95% sequence identity to the nucleic acid sequence of SEQ ID NO. 11.

217. The composition of claim 216, wherein the transgene encodes a polynucleotide has the nucleic acid sequence of SEQ ID NO. 11.

218. The composition of claim any one of claims 195-197, wherein the GBA comprises a signal peptide.

219. The composition of claim 218, wherein the signal peptide has an amino acid sequence that is at least 85% identical to the amino acid sequence of SEQ ID. NO. 16.

220. The composition of claim 219, wherein the signal peptide has an amino acid sequence that is at least 90% identical to the amino acid sequence of SEQ ID. NO. 16.

221. The composition of claim 220, wherein the signal peptide has an amino acid sequence that is at least 95% identical to the amino acid sequence of SEQ ID. NO. 16.

222. The composition of claim 221, wherein the signal peptide has the amino acid of SEQ ID. NO. 16.

223. The composition of claim any one of claims 195-197, wherein the signal peptide is encoded by polynucleotide having a nucleic acid sequence that is at least 85% identity to the nucleic acid sequence of SEQ ID NO. 20.

224. The composition of claim 223, wherein the signal peptide is encoded by polynucleotide having a nucleic acid sequence that is at least 90% identity to the nucleic acid sequence of SEQ ID NO. 20.

225. The composition of claim 224, wherein the signal peptide is encoded by polynucleotide having a nucleic acid sequence that is at least 95% identity to the nucleic acid sequence of SEQ ID NO. 20.

226. The composition of claim 225, wherein the signal peptide is encoded by polynucleotide having a nucleic acid of SEQ ID NO. 20.

227. The composition of claim 195, wherein the transgene encoding secreted GBA comprises a secretory signal peptide.

228. The composition of claim 227, wherein the signal peptide is a GBA signal peptide.

229. The method of claim 227, wherein the secretory signal peptide is an alpha-1 antitrypsin secretory signal peptide.

230. The composition of claim 227, wherein the secretory signal peptide is an insulin-like growth factor II (IGF-II) secretory signal peptide.

231. The composition of claim any one of claims 195-230, wherein the transgene encodes a GBA fusion protein.

232. The composition of claim 231, wherein the GBA fusion protein has an amino acid sequence that is at least 85% identical to the amino acid sequence of SEQ ID NO. 2.

233. The composition of claim 232, wherein the GBA fusion protein has an amino acid sequence that is at least 90% identical to the amino acid sequence of SEQ ID NO. 2.

234. The composition of claim 233, wherein the GBA fusion protein has an amino acid sequence that is at least 95% identical to the amino acid sequence of SEQ ID NO. 2.

235. The composition of claim 234, wherein the GBA fusion protein has the amino acid sequence of SEQ ID NO. 2.

236. The composition of claim 231, wherein the GBA fusion protein has an amino acid sequence that is at least 85% identical to the amino acid sequence of SEQ ID NO. 3.

237. The composition of claim 236, wherein the GBA fusion protein has an amino acid sequence that is at least 90% identical to the amino acid sequence of SEQ ID NO. 3.

238. The composition of claim 237, wherein the GBA fusion protein has an amino acid sequence that is at least 95% identical to the amino acid sequence of SEQ ID NO. 3.

239. The composition of claim 238, wherein the GBA fusion protein has the amino acid sequence of SEQ ID NO. 3.

240. The composition of claim 231, wherein the GBA fusion protein has an amino acid sequence that is at least 85% identical to the amino acid sequence of SEQ ID NO. 4.

241. The composition of claim 240, wherein the GBA fusion protein has an amino acid sequence that is at least 90% identical to the amino acid sequence of SEQ ID NO. 4.

242. The composition of claim 241, wherein the GBA fusion protein has an amino acid sequence that is at least 95% identical to the amino acid sequence of SEQ ID NO. 4.

243. The composition of claim 242, wherein the GBA fusion protein has the amino acid sequence of SEQ ID NO. 4.

244. The composition of claim 231, wherein the transgene encoding the GBA fusion protein has a nucleic acid sequence that is at least 85% identical to the nucleic acid sequence of SEQ ID NO. 8.

245. The composition of claim 244, wherein the transgene encoding the GBA fusion protein has a nucleic acid sequence that is at least 90% identical to the nucleic acid sequence of SEQ ID NO. 8.

246. The composition of claim 245, wherein the transgene encoding the GBA fusion protein has a nucleic acid sequence that is at least 95% identical to the nucleic acid sequence of SEQ ID NO. 8.

247. The composition of claim 246, wherein the transgene encoding the GBA fusion protein has a nucleic acid sequence of SEQ ID NO. 8.

248. The composition of claim 231, wherein the transgene encoding the GBA fusion protein has a nucleic acid sequence that is at least 85% identical to the nucleic acid sequence of SEQ ID NO. 9.

249. The composition of claim 248, wherein the transgene encoding the GBA fusion protein has a nucleic acid sequence that is at least 90% identical to the nucleic acid sequence of SEQ ID NO. 9.

250. The composition of claim 249, wherein the transgene encoding the GBA fusion protein has a nucleic acid sequence that is at least 95% identical to the nucleic acid sequence of SEQ ID NO. 9.

251. The composition of claim 250, wherein the transgene encoding the GBA fusion protein has a nucleic acid sequence of SEQ ID NO. 9.

252. The composition of claim 231, wherein the transgene encoding the GBA fusion protein has a nucleic acid sequence that is at least 85% identical to the nucleic acid sequence of SEQ ID NO. 10.

253. The composition of claim 252, wherein the transgene encoding the GBA fusion protein has a nucleic acid sequence that is at least 90% identical to the nucleic acid sequence of SEQ ID NO. 10.

254. The composition of claim 253, wherein the transgene encoding the GBA fusion protein has a nucleic acid sequence that is at least 95% identical to the nucleic acid sequence of SEQ ID NO. 10.

255. The composition of claim 254, wherein the transgene encoding the GBA fusion protein has a nucleic acid sequence of SEQ ID NO. 10.

256. The composition of any one of claims 231-255, wherein the GBA fusion protein comprises GBA and a glycosylation independent lysosomal targeting (GILT) tag.

257. The composition of claim 256, wherein the GILT tag comprises a human IGF-II mutein having an amino acid sequence that is at least 70% identical to the amino acid sequence of mature human IGF-II (SEQ ID NO. 12), and having diminished binding affinity for the insulin receptor relative to the affinity of naturally-occurring human IGF-II for the insulin receptor, wherein the IGF-II mutein is resistant to furin cleavage and binds to the human cation-independent mannose-6-phosphate receptor in a mannose-6-phosphate-independent manner.

258. The composition of claim 257, wherein the IGF-II mutein comprises a mutation within a region corresponding to amino acids 30-40 of SEQ ID NO. 12, and wherein the mutation abolishes at least one furin protease cleavage site.

259. The composition of claim 258, wherein the mutation is an amino acid substitution, deletion, and/or insertion.

260. The composition of claim 259, wherein the mutation is a Lys or Ala amino acid substitution at a position corresponding to Arg37 or Arg40 of SEQ ID NO. 12.

261. The composition of claim 260, wherein the mutation is a deletion or replacement of amino acid residues corresponding to positions selected form the group consisting of 31-40, 32-40, 33-40, 34-40, 30-39, 31-39, 32-39, 34-37, 33-39, 35-39, 36-39, 37-40, 34-40 of SEQ ID NO. 12, and combinations thereof.

262. The composition of any one claims 256-261, wherein the GILT tag has an amino acid sequence that is at least 70% identical to the amino acid sequence of SEQ ID NO. 13.

263. The composition of claim 262, wherein the GILT tag has an amino acid sequence that is at least 80% identical to the amino acid sequence of SEQ ID NO. 13.

264. The composition of claim 263, wherein the GILT tag has an amino acid sequence that is at least 90% identical to the amino acid sequence of SEQ ID NO. 13.

265. The composition of claim 264, wherein the GILT tag has the amino acid sequence of SEQ ID NO. 13.

266. The composition of any one claims 256-261, wherein the GILT tag has an amino acid sequence that is at least 70% identical to the amino acid sequence of SEQ ID NO. 14.

267. The composition of claim 266, wherein the GILT tag has an amino acid sequence that is at least 80% identical to the amino acid sequence of SEQ ID NO. 14.

268. The composition of claim 267, wherein the GILT tag has an amino acid sequence that is at least 90% identical to the amino acid sequence of SEQ ID NO. 14.

269. The composition of claim 268, wherein the GILT tag has the amino acid sequence of SEQ ID NO. 14.

270. The composition of any one claims 256-261, wherein the GILT tag has an amino acid sequence that is at least 70% identical to the amino acid sequence of SEQ ID NO. 15.

271. The composition of claim 270, wherein the GILT tag has an amino acid sequence that is at least 80% identical to the amino acid sequence of SEQ ID NO. 15.

272. The composition of claim 271, wherein the GILT tag has an amino acid sequence that is at least 90% identical to the amino acid sequence of SEQ ID NO. 15.

273. The composition of claim 272, wherein the GILT tag has the amino acid sequence of SEQ ID NO. 15.

274. The composition of any one of claims 256-273, wherein the GILT tag is encoded by a polynucleotide having a nucleic acid sequence that is at least 85% identical to the nucleic acid sequence of SEQ ID NO. 17.

275. The composition of claim 274, wherein the GILT tag is encoded by a polynucleotide having a nucleic acid sequence that is at least 90% identical to the nucleic acid sequence of SEQ ID NO. 17.

276. The composition of claim 275, wherein the GILT tag is encoded by a polynucleotide having a nucleic acid sequence that is at least 95% identical to the nucleic acid sequence of SEQ ID NO. 17.

277. The composition of claim 276, wherein the GILT tag is encoded by a polynucleotide having the nucleic acid sequence of SEQ ID NO. 17.

278. The composition of any one claims 256-273, wherein the GILT tag is encoded by a polynucleotide having a nucleic acid sequence that is at least 85% identical to the nucleic acid sequence of SEQ ID NO. 18.

279. The composition of claim 278, wherein the GILT tag is encoded by a polynucleotide having a nucleic acid sequence that is at least 90% identical to the nucleic acid sequence of SEQ ID NO. 18.

280. The composition of claim 279, wherein the GILT tag is encoded by a polynucleotide having a nucleic acid sequence that is at least 95% identical to the nucleic acid sequence of SEQ ID NO. 18.

281. The composition of claim 280, wherein the GILT tag is encoded by a polynucleotide having the nucleic acid sequence of SEQ ID NO. 18.

282. The composition of any one claims 256-273, wherein the GILT tag is encoded by a polynucleotide having a nucleic acid sequence that is at least 85% identical to the nucleic acid sequence of SEQ ID NO. 19.

283. The composition of claim 282, wherein the GILT tag is encoded by a polynucleotide having a nucleic acid sequence that is at least 90% identical to the nucleic acid sequence of SEQ ID NO. 19.

284. The composition of claim 283, wherein the GILT tag is encoded by a polynucleotide having a nucleic acid sequence that is at least 95% identical to the nucleic acid sequence of SEQ ID NO. 19.

285. The composition of claim 284, wherein the GILT tag is encoded by a polynucleotide having the nucleic acid sequence of SEQ ID NO. 19.

286. The composition of any one of claims 194-285, wherein the GBA fusion protein comprises a receptor-binding (Rb) domain of apolipoprotein E (ApoE).

287. The composition of claim 286, wherein the Rb domain comprises a portion of ApoE having the amino acid sequence of residues 25-185, 50-180, 75-175, 100-170, 125-160, or 130-150 of SEQ ID NO. 21.

288. The composition of claim 286, wherein the Rb domain comprises a region having at least 70% sequence identity to the amino acid sequence of residues 159-167 of SEQ ID NO. 21.

289. The composition of any one of claims 194-288, wherein the transgene encoding GBA further comprises a micro RNA (miRNA) targeting sequence in the 3′-UTR.

290. The composition of claim 289, wherein the miRNA targeting sequence is a miR-126 targeting sequence.

291. The composition of claim 194 or 195, wherein the pluripotent cells are CD34+ cells.

292. The composition of claim 291, wherein the CD34+ cells are embryonic stem cells.

293. The composition of claim 291, wherein the CD34+ cells are induced pluripotent stem cells.

294. The composition of claim 291, wherein the CD34+ cells are hematopoietic stem cells.

295. The composition of claim 291, wherein the CD34+ cells are myeloid progenitor cells.

296. The composition of any one of claims 194-295, wherein at least one of the one or more transgenes that each encode an M2-promoting agent encodes a cytokine selected from the group consisting of IL-25, IL-4, IL-10, IL-13, and TGF-β.

297. The composition of claim 296, wherein at least one of the one or more transgenes that each encode an M2-promoting agent encodes IL-25.

298. The composition of any one of claims 194-297, wherein at least one of the one or more transgenes that each encode an M2-promoting agent encodes an agent selected from the group consisting of a glucocorticoid receptor, a PPAR, PPARγ, PPARβ/δ, an estrogen receptor, NR4A2, KDM6B, MSX3, FAM19A3, NF-κB p50, miR124, miR21, and miR181c, CX3CL1, CX3CR1, CD200, CD200R, CFH, CD47, CD55, CD46, ADGRE5, SIRPα, and siglecs.

299. The composition of any one of claims 194-298, wherein the pluripotent cells are transduced ex vivo to express the GBA and the one or more M2-promoting agents.

300. The composition of any one of claims 194-298, wherein the pluripotent cells are transfected ex vivo to express the GBA and the one or more M2-promoting agents.

301. The composition of any one of claims 194-300, wherein endogenous GBA is disrupted in the pluripotent cells.

302. A kit comprising the composition of any one of claims 194-301 and a package insert.

303. The kit of claim 302, wherein the package insert instructs a user of the kit to perform the method of any one of claims 1-193.
